(19) United States
(12) Patent Application Publication
(10) Pub. No.: US 2008/0064053 A1
(43) Pub. Date: Mar. 13, 2008

(54) Genotoxicity Testing

(76) Inventors: Richard Maurice Walmsley, Manchester (GB); Nicholas Billinton, Manchester (GB)

Correspondence Address: HOGAN & HARTSON L.L.P., 1999 AVENUE OF THE STARS, SUITE 1400, LOS ANGELES, CA, 90067, US

(21) Appl. No.: 10/565,821

(22) PCT Filed: Jul. 30, 2004
(86) PCT No.: PCT/GB04/03337

§ 371 Date: Jan. 24, 2006

(30) Foreign Application Priority Data
Jul. 31, 2003 (GB) 0317944.7
Jul. 31, 2003 (GB) 0317945.4

(51) Int. Cl. C12Q 1/02; C12N 1/00; C12N 15/00

(52) U.S. Cl. 435/29; 435/254.2; 435/254.21; 435/320.1

(57) ABSTRACT

A recombinant vector comprises a recombinant DNA molecule comprising a DNA sequence that encodes a light emitting reporter protein. The DNA sequence is operatively linked to a regulatory element arranged to activate expression of the DNA sequence in response to DNA damage. When used to transform a cell, the vector does not substantially alter the sensitivity of the cell to geneticin, when compared to the sensitivity of the cell which has not been transformed with the vector.

The present invention relates to improved methods for detecting agents that cause or potentiate DNA damage and to molecules and transformed cells that may be usefully employed in such methods.

DNA damage is induced by a variety of agents such as ultraviolet light, X rays, free radicals, methylating agents and other mutagenic compounds. These agents may cause damage to the DNA that comprises the genetic code of an organism and cause mutations in genes. In microorganisms such mutations may lead to the evolution of new undesirable strains of the microorganism. For instance, antibiotic or herbicide resistant bacteria may arise. In animals these mutations can lead to carcinogenesis or may damage the gametes to give rise to congenital defects in offspring.

These DNA damaging agents may chemically modify the nucleotides that comprise DNA and may also break the phosphodiester bonds that link the nucleotides or disrupt association between bases (T-A or C-G). To counter the effect of these DNA damaging agents cells have evolved a number of mechanisms. For instance the SOS response in E. coli is a well-characterised cellular response induced by DNA damage in which a series of proteins are expressed, including DNA repair enzymes, which repair the damaged DNA.

There are numerous circumstances when it is important to identify what agents may cause or potentiate DNA damage. It is particularly important to detect agents that cause DNA damage when assessing whether it is safe to expose a person to these agents. For instance a method of detecting these agents may be used as a mutagenesis assay for screening compounds that are candidate medicaments, food additives or cosmetics to assess whether or not the compound of interest induces DNA damage. Alternatively, methods of detecting DNA damaging agents may be used to monitor for contamination of water supplies with pollutants that contain mutagenic compounds.

Various methods, such as the Ames Test, for determining the toxicity of an agent are known but are unsatisfactory for a number of reasons. For instance, incubation of samples can take many days when it is often desirable to obtain genotoxic data in a shorter time frame. Furthermore, many known methods of detecting DNA damage (including the Ames Test and related methods) assay lasting DNA damage, as an endpoint, either in the form of misrepaired DNA (mutations and recombinations) or unrepaired damage in the form of fragmented DNA. However most DNA damage is repaired before such an endpoint can be measured and lasting DNA damage only occurs if the conditions are so severe that the repair mechanisms have been saturated.

An improved genotoxicity test is disclosed in WO 98/44149. This specification concerns recombinant DNA molecules comprising a regulatory element that activates gene expression in response to DNA damage operatively linked to a DNA sequence that encodes a light emitting reporter protein. Such DNA molecules may be used to transform a cell and such cells used in a genotoxic test for detecting for the presence of an agent that causes or potentiates DNA damage. The cells may be subjected to an agent and the expression of the light emitting reporter protein from the cell indicates that the agents cause DNA damage.

The genotoxicity tests described in WO 98/44149 detect the induction of DNA repair activity, which evolved to minimise conversion of damage to mutation. The method described in WO 98/44149 may therefore be used to detect for the presence of DNA damaging agents.

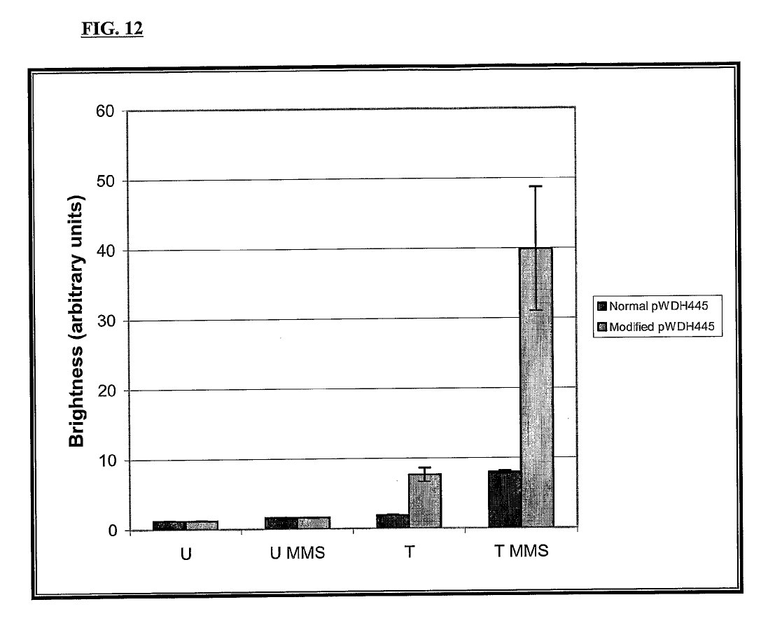

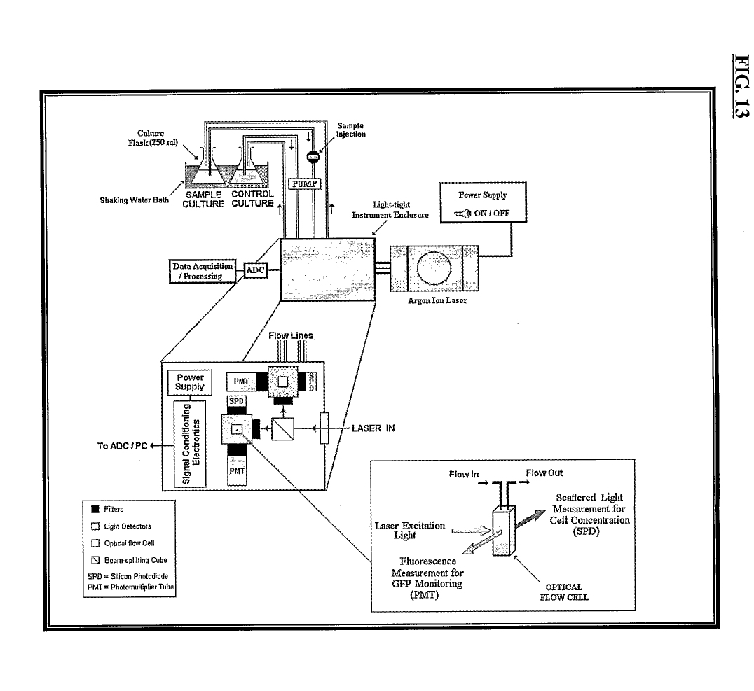

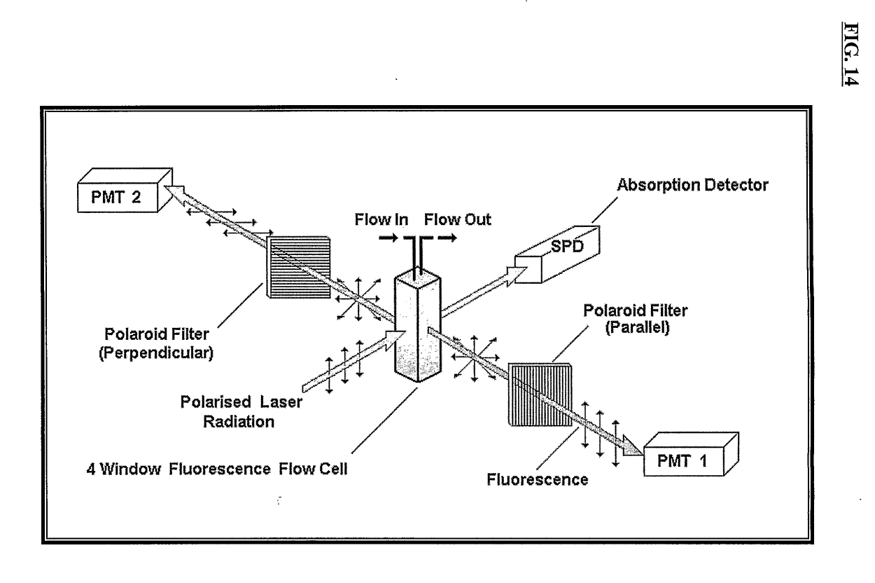

WO 98/44149 describes a number of useful genetic constructs that may be used to transform a cell such that it may be used in a genotoxic test. One such construct is yEGFP-444 (illustrated in FIG. 12 of WO 98/44149), and the present invention is based upon a novel type of construct that was identified during developmental work carried out with yEGFP-444.

According to a first aspect of the present invention, there is provided a recombinant vector comprising a recombinant DNA molecule comprising a DNA sequence that encodes a light emitting reporter protein, which DNA sequence is operatively linked to a regulatory element arranged to activate expression of the DNA sequence in response to DNA damage, wherein when used to transform a cell, the vector does not substantially alter the sensitivity of the cell to geneticin, when compared to the sensitivity of the cell which has not been transformed with the vector.

According to a second aspect of the invention, there is provided a method of generating a recombinant vector, the method comprising the steps of:
(i) providing a vector backbone with a DNA sequence that encodes a light emitting reporter protein;
(ii) operatively linking the DNA sequence to a regulatory element arranged to activate expression of the DNA sequence in response to DNA damage;
(iii) providing the vector backbone with a selectable marker arranged to confer resistance to geneticin; and
(iv) rendering the selectable marker non-functional, wherein when used to transform a cell, the vector does not substantially alter the sensitivity of the cell to geneticin, when compared to the sensitivity of the cell which has not been transformed with the vector.

According to a third aspect of the invention, there is provided a cell containing a recombinant vector in accordance with the first aspect of the present invention, or made according to a method of the second aspect of the invention.

According to a fourth aspect of the present invention, there is provided a method of detecting for the presence of an agent that causes or potentiates DNA damage comprising subjecting a cell in accordance with the third aspect of the present invention to an agent and monitoring the expression of the light emitting reporter protein from the cell.

By “regulatory element” we mean a DNA sequence that regulates the transcription of a gene with which it is associated.

By “operatively linked” we mean that the regulatory element is able to induce the expression of the reporter protein.

By “reporter protein” we mean a protein which when expressed in response to the regulatory element of the DNA molecule of the invention is detectable by means of a suitable assay procedure.

By “pWDH445” we mean the expression vector illustrated in FIG. 5 of this specification. pWDH445 was originally called yEGFP444 and was originally disclosed in FIG. 12 of WO 97/44149.

By the term “does not substantially alter the sensitivity of the cell to geneticin”, we mean that the sensitivity to geneticin of a cell which has been transformed with the vector according to the first aspect of the invention is at least 70% that of the sensitivity to geneticin of a cell which has not been transformed with the vector. Preferably, the sensitivity to geneticin is at least 80%, more preferably, at least 90%, even more preferably at least 95%, and most preferably, at least 99% that of the sensitivity to geneticin of a cell which has not been transformed with the vector. It is especially preferred that the sensitivity to geneticin of a cell which has been transformed with the vector according to the first aspect is the same as that of the sensitivity to geneticin of a cell which has not been transformed with the vector. Conversely, the resistance to geneticin of a cell transformed with the vector is the same as that of the resistance to geneticin of the cell which has not been transformed with the vector.

The method of the fourth aspect of the invention represents a novel cost-effective genotoxicity screen, that may be used to provide a pre-regulatory screening assay for use by the pharmaceutical industry and in other applications where significant numbers of compounds need to be tested. It provides a higher throughput and a lower compound consumption than existing eukaryotic genotoxicity assays and is sensitive to a broad spectrum of mutagens and, importantly, clastogens.

The method of the fourth aspect of the invention is suitable for assessing whether or not an agent may cause DNA damage. It is particularly useful for detecting agents that cause DNA damage when assessing whether it is safe to expose a person to DNA damaging agents. For instance, the method may be used as a mutagenesis assay for screening whether or not known agents, such as candidate medicaments, foodstuffs or cosmetics, induce DNA damage. Alternatively, the method of the fourth aspect of the invention may be used to monitor for contamination of water supplies with pollutants containing DNA damaging agents.

The method of the fourth aspect of the invention may equally be used for assessing whether an agent may potentiate DNA damage. For example, certain agents can cause accumulation of DNA damage by inhibiting DNA repair (for instance by preventing expression or function of a repair protein) without directly inflicting DNA damage. These agents are often known as co-mutagens.

The present invention is based upon work conducted on the vectors disclosed in WO 98/44149. The inventors have found that a spontaneous rearrangement of the known vector pWDH445 occurred that resulted in a surprisingly brighter reporter.

By the term “brighter reporter”, we mean the degree of fluorescence from the reporter is surprisingly at least double that of the known vector pWDH445, more preferably at least 3 times, at least 5 times, at least 7 times, at least 10 times, and most preferably, at least 12 times that of the known vector pWDH445.

The inventors therefore conducted experiments, which are described in more detail in the Examples, to characterise this fortuitous mutation and to isolate recombinant vectors according to the first aspect of the present invention.

The vector pWDH445 is illustrated in FIG. 5. The inventors have found that, surprisingly, loss of the kanMX3 module function (i.e. removing geneticin resistance) from the pWDH445 plasmid results in a vector capable of expressing the reporter protein (GFP in the case of pWDH445) such that the signal from the reporter is significantly greater when DNA damage occurs. The kanMX3 module may be disrupted such that a mutation occurs by means of deletion, substitution or addition of nucleotides provided that geneticin resistance is impaired. Hence, the vector may comprise nucleotide bases of the kanMX3 module, but the gene is disrupted such that it is non-functional. Hence, the vector according to the first aspect preferably comprises a light emitting reporter protein, a regulatory element and a non-functional kanMX module.

Discovery of the loss of kanMX3 module function in vector pWDH445 led the inventors to realise that vectors according to the first aspect of the invention were particularly useful for genotoxicity testing. They also devised the method according to the second aspect for generating preferred vectors according to the first aspect, which vectors may be used in a method according to the fourth aspect.

Hence, the vector backbone used in the method of the second aspect may comprise any suitable vector backbone known to those skilled in the art, which may be used to carry the light emitting reporter protein, the regulatory element, and the selectable marker conferring resistance to geneticin. The backbone may comprise a low copy number or high copy number plasmid. The backbone may be selected from the preferably well-known vectors pWDH445, pWDH443 (Walmsley et al, R. M., 1997), pRS316 (Sikorski et al., 1989).

Recombinant vectors according to the first aspect may be designed such that they may autonomously replicate in the nucleus of the cell. In this case, elements, which induce DNA replication, may be required in the recombinant vector. Hence, the vector may comprise an origin of replication, preferably, for yeast. Suitable origins of replication will be known to the skilled technician. For example, a suitable element is derived from the yeast 2 μl plasmid. Such replicating vectors can give rise to multiple copies of the DNA molecule in a transformant cell and are therefore useful when over-expression (and thereby increased light emission) of the reporter protein is required. The transformant cell will be the cell according to the third aspect.

The recombinant vector may comprise at least one selectable marker to enable selection of cells transfected with the vector, and preferably, to enable selection of cells harbouring the recombinant vector that incorporates the DNA molecule of the first aspect. Examples of suitable selectable markers include genes conferring resistance to an antibiotic, for example, kanamycin, and ampicillin etc. Alternatively, or additionally, selectable markers may include auxotrophic markers, i.e. those which restore prototrophy, for example, yeast URA3 or LEU2 genes.

The selectable marker conferring geneticin resistance may comprise any of the kanMX modules, for example, kanMX2, kanMX3, kanMX4, kanMX6. However, it is preferred that the selectable marker comprises the kanMX3 module. Therefore, preferred vectors according to the first aspect of the invention comprise pWDH445 (y EGFP-444 in WO 98/44149) with disruption of the kanMX3 module.

The DNA sequence that encodes a light emitting reporter protein may code for any light emitting protein. However, it is preferred that the DNA sequence codes for a protein that is fluorescent. Preferred DNA sequences that encode a light emitting reporter protein code for Green Fluorescent Protein (GFP) and light emitting derivatives thereof. GFP is from the jellyfish Aequorea victoria and is able to absorb blue light and re-emits an easily detectable green light and is thus suitable as a reporter protein. GFP may be advantageously used as a reporter protein because its measurement is simple and reagent free and the protein is non-toxic.

Derivatives of GFP include DNA sequences encoding for polypeptide analogues or polypeptide fragments of GFP, which are able to emit light. Many of these derivatives absorb and re-emit light at wavelengths different to GFP found endogenously in Aequorea victoria. For instance, preferred DNA molecules according to the first aspect of the invention have a DNA sequence that encodes the S65T derivative of GFP (in which serine 65 of GFP is replaced by a threonine). S65T GFP has the advantage that it is brighter than wild-type GFP (when excited at its longest-wavelength peak) and shows only slow photobleaching. Furthermore, S65T GFP produces a good quantum yield of fluorescence and matches the output of argon ion lasers used in fluorescence activated cell sorters. Cells according to the second aspect of the invention which contain DNA molecules coding S65T GFP may be used according to the method of the fourth aspect of the invention and are particularly useful when light emission is measured from cell extracts.

A most preferred DNA sequence encodes for a yeast enhanced GFP (yEGFP) such as the GFP derivative described by Cormack et al. (1997) (in Microbiology 143 p 303-311). Such yEGFP has an amino acid sequence, which is biased for usage in yeast. Thus yEGFP is particularly suitable for transforming cells according to the second aspect of the invention which are yeast. Furthermore we have found that light emitted from yEGFP in such yeast is even greater than that emitted by S65T derivatives. Recombinant DNA molecules coding yEGFP are also useful because yEGFP is less heat sensitive than nascent GFP.

It is preferred that the regulatory element of the recombinant DNA molecule activates expression of the reporter protein when DNA damage occurs. Such regulatory elements ideally comprise a promoter sequence, which induces RNA polymerase to bind to the DNA molecule and start transcribing the DNA encoding the reporter protein. The regulatory element may also comprise other functional DNA sequences such as translation initiation sequences for ribosome binding or DNA sequences that bind transcription factors which promote gene expression following DNA damage. Regulatory elements may even code for proteins, which act to dislodge inhibitors of transcription from the regulated gene and thereby increase transcription of that gene.

Preferred regulatory elements are DNA sequences that are associated in nature with the regulation of the expression of DNA repair proteins. For instance, the regulatory elements from genes such as RAD2, RAD6, RAD7, RAD18, RAD23, RAD51, RAD54, CDC7, CDC8, CDC9, MAG1, PHR1, DIN1, DDR48 and UB14 from yeast may be used to make recombinant DNA molecules according to the first aspect of the invention. Hence, the regulatory element used in the method of the second aspect may comprise genes such as RAD2, RAD6, RAD7, RAD18, RAD23, RAD51, RAD54, CDC7, CDC8, CDC9, MAG1, PHR1, DIN1, DDR48 or UB14 from yeast.

A preferred regulatory element comprises the promoter and 5′ regulatory sequences of the RAD54 repair gene. Such a regulatory element may be derived from yeast and particularly Saccharomyces cerevisiae. It is most preferred that the regulatory element comprises the promoter and 5′ regulatory sequences of the RAD54 repair gene which correspond to the DNA sequence identified in WO 98/44149 or a functional analogue or fragment thereof.

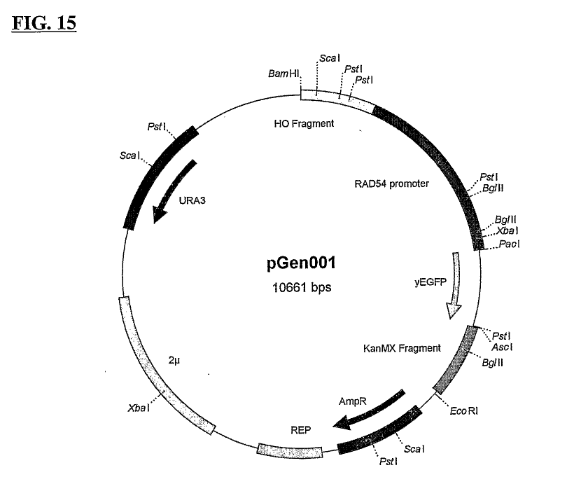

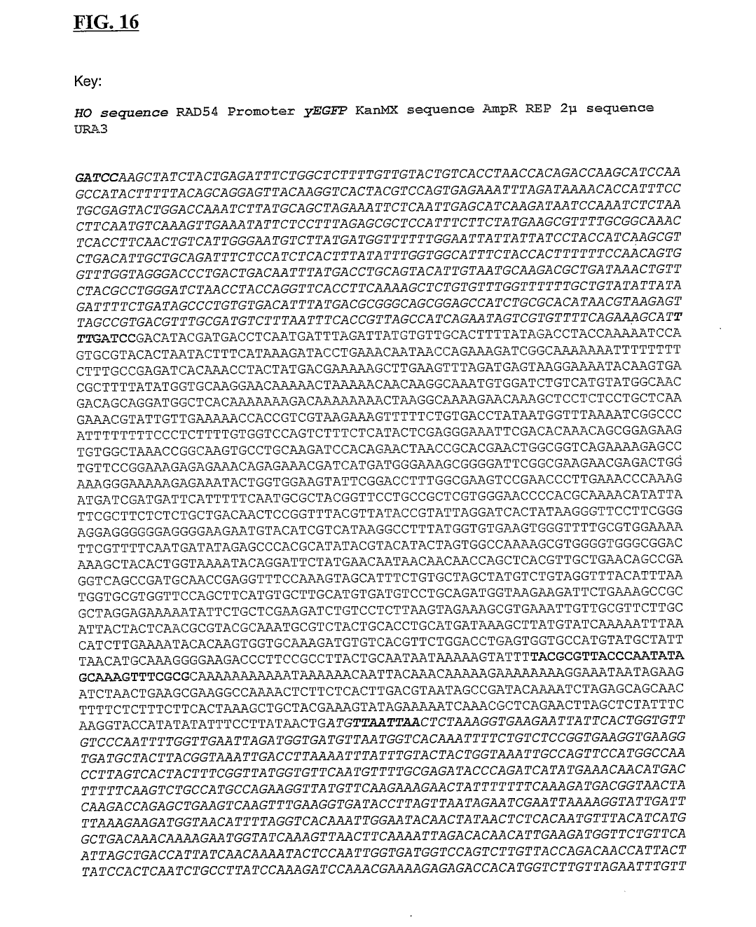

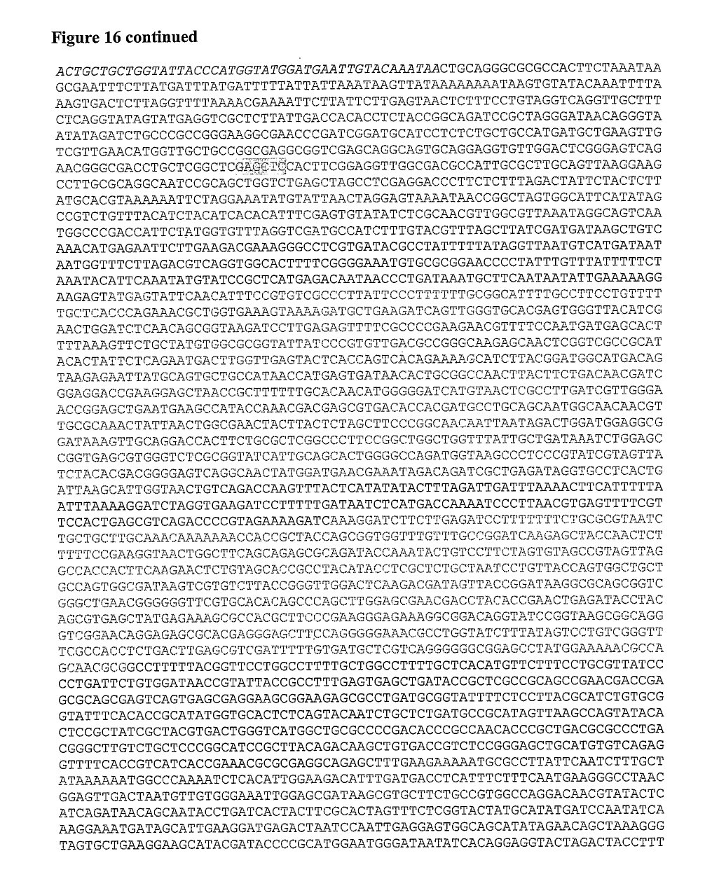

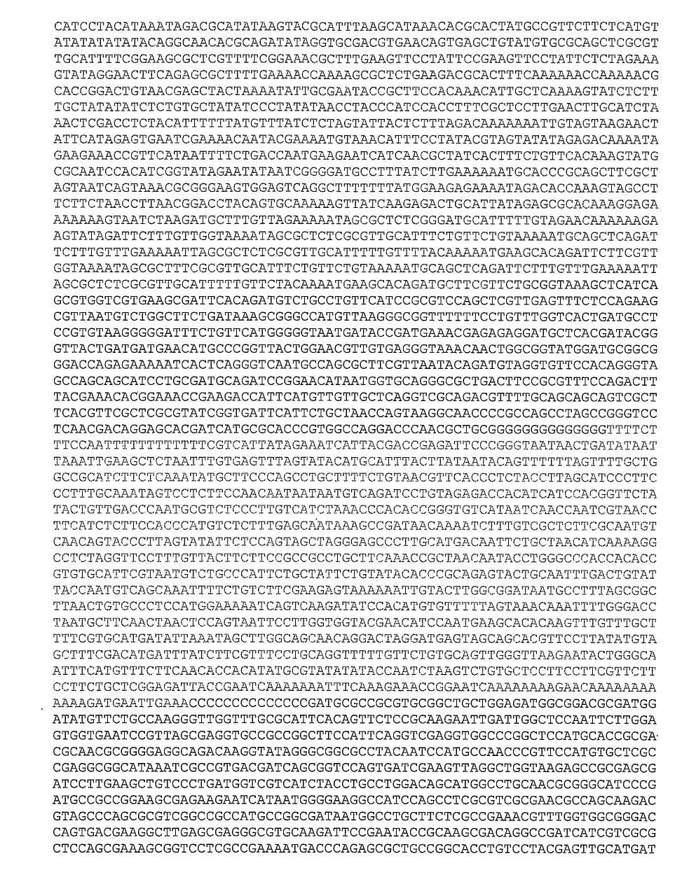

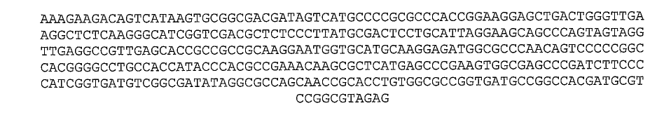

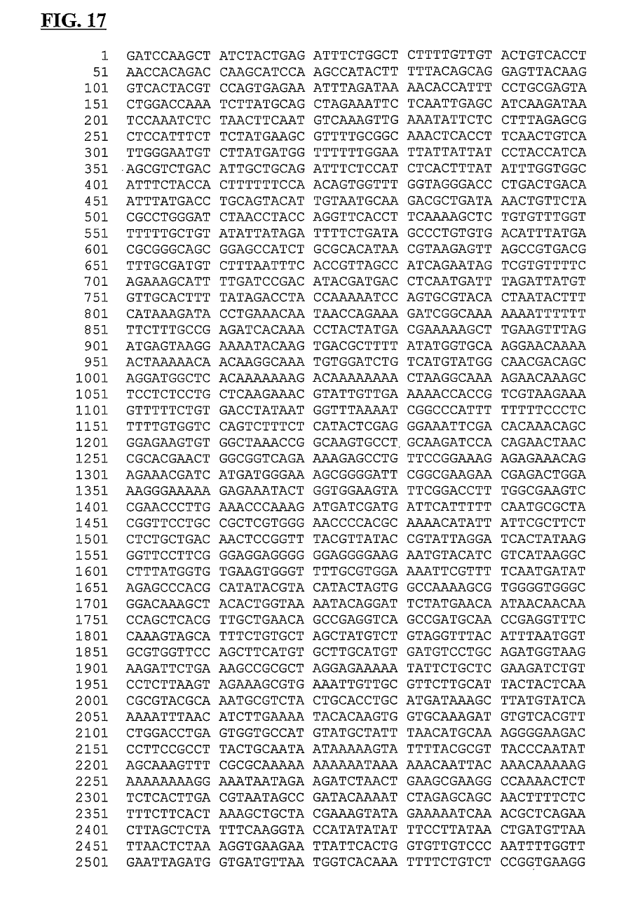

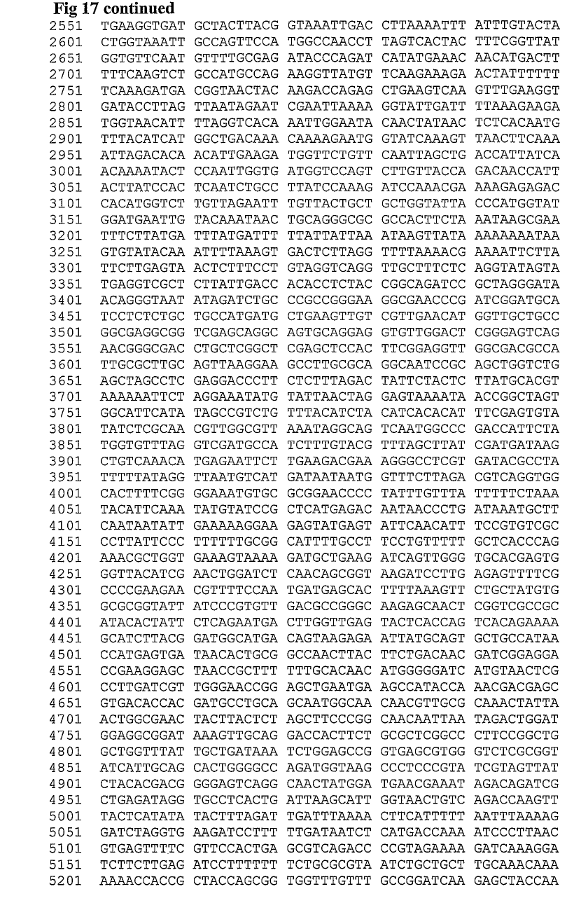

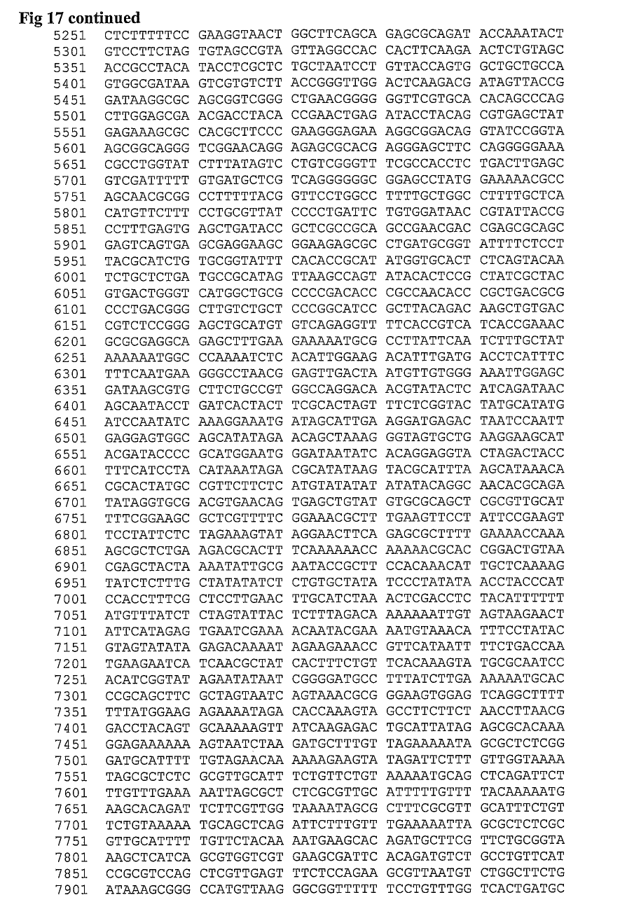

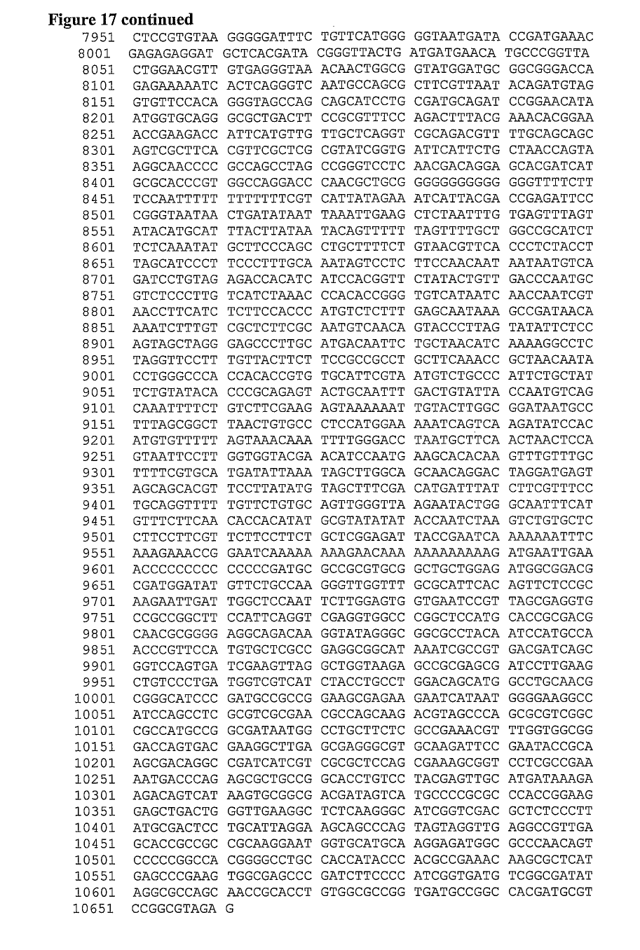

Therefore, most preferred recombinant DNA molecules comprise a RAD54 regulatory element operatively linked to a DNA sequence that encodes a GFP or light emitting derivative thereof. Accordingly, a preferred recombinant vector according to the first aspect comprises the RAD54 gene operatively linked to GFP, or light emitting derivative thereof. It is especially preferred that the vector additionally comprises a non-functional kanMX3 module. Hence, most preferred recombinant vector according to the first aspect of the invention is pGen001, as illustrated in FIG. 15. The nucleotide sequence of pGen001 is given in FIGS. 16 and 17.

It will be appreciated that the recombinant DNA molecule in pGen001 is interchangeable. Accordingly, the RAD54 promoter—yEGFP expression cassette depicted in FIG. 15 may be easily replaced (e.g. by Bam H1/Asc1 digestion) to insert another recombinant DNA molecule according to the invention (see below).

The inventors have also found that the RAD54 regulatory element in pGen001 may be replaced by an RNR regulatory element to provide a recombinant vector according to the first aspect. The resultant recombinant vector produces a surprisingly brighter signal of the light emitting reporter protein, when exposed to DNA damage. Hence, the regulatory element of the recombinant vector according to the first aspect, and as used in the method of the second aspect, may comprise an RNR regulatory element.

By “RNR regulatory element”, we mean a DNA sequence that is the natural regulator of an RNR gene.

Preferred vectors according to the invention comprise pWDH445 (yEGFP-444 in WO 98/44149) with disruption of the kanMX3 module in which an RNR regulatory element replaces the RAD54 regulatory element. The kanMX3 module may be disrupted such that a mutation occurs by means of deletion, substitution or addition of nucleotides provided that geneticin resistance is impaired.

The RNR regulatory element of the recombinant DNA molecule activates expression of the reporter protein when DNA damage occurs. Such regulatory elements ideally comprise a promoter sequence, which induces RNA polymerase to bind to the DNA molecule and start transcribing the DNA encoding for the reporter protein. The regulatory element may also comprise other functional DNA sequences such as translation initiation sequences for ribosome binding or DNA sequences that bind transcription factors which promote gene expression following DNA damage. Regulatory elements may even code for proteins which act to dislodge inhibitors of transcription from the regulated gene and thereby increase transcription of that gene.

Preferred regulatory elements are DNA sequences that are associated in nature with the regulation of the expression of RNR DNA repair proteins. For instance, the regulatory elements from genes such as RNR1, RNR2 and RNR3 from yeast may be used in the method of the second aspect to make recombinant DNA molecules according to the first aspect of the invention. Hence, the regulatory element may comprise an RNR1, RNR2 or RNR3 gene from yeast.

A preferred regulatory element comprises the promoter and 5′ regulatory sequences of the RNR2 gene. The RNR2 gene may be found on chromosome X of Saccharomyces cerevisiae. A preferred regulatory element may be derived from between co-ordinates 387100 and 398299 associated with the RNR2 gene on chromosome X as identified in the Saccharomyces cerevisiae genome database. It is more preferably derived from between co-ordinates 387100 and 393299. The database may be accessed by the World Wide Web at many sites. For example at genome-www.stanford.edu.

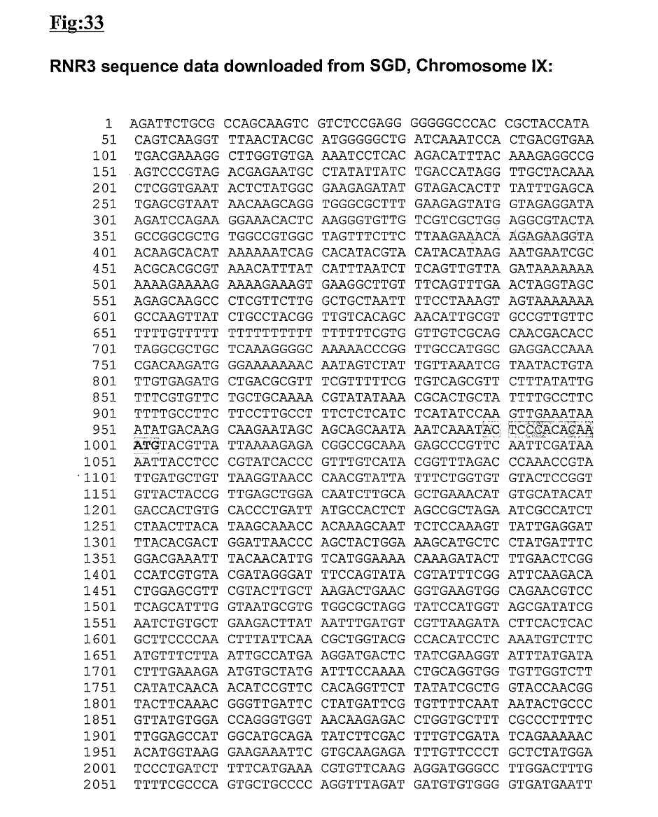

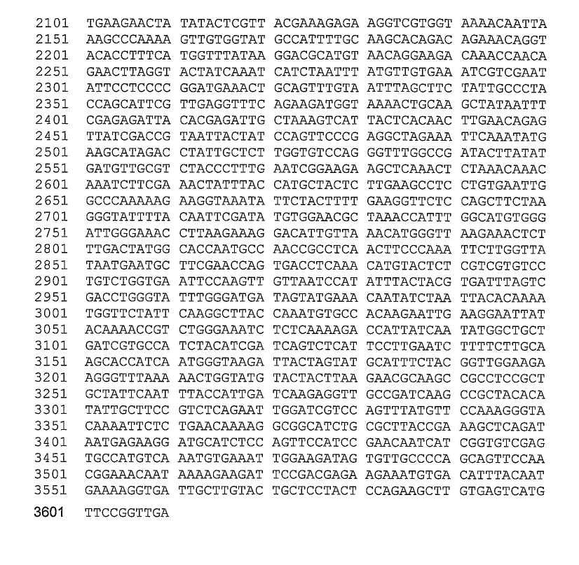

The regulatory element of RNR3 is particularly preferred. The sequence of this RNR3 element is well known and is illustrated in FIG. 33. The gene is YIL066C, and the FIG. 33 also shows 1000 bp upstream of the ATG start codon, which is highlighted in bold. The total sequence length of RNR3 is 2610 bp long. The RNR3 promoter is particularly suitable because its induction is DNA damage specific; there is low level expression under normal condition; and significant induction incurs in response to damage.

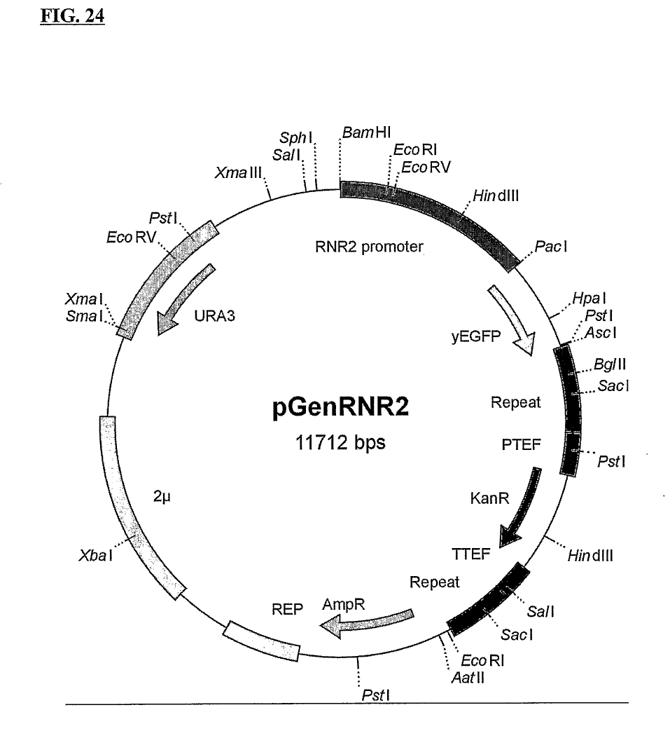

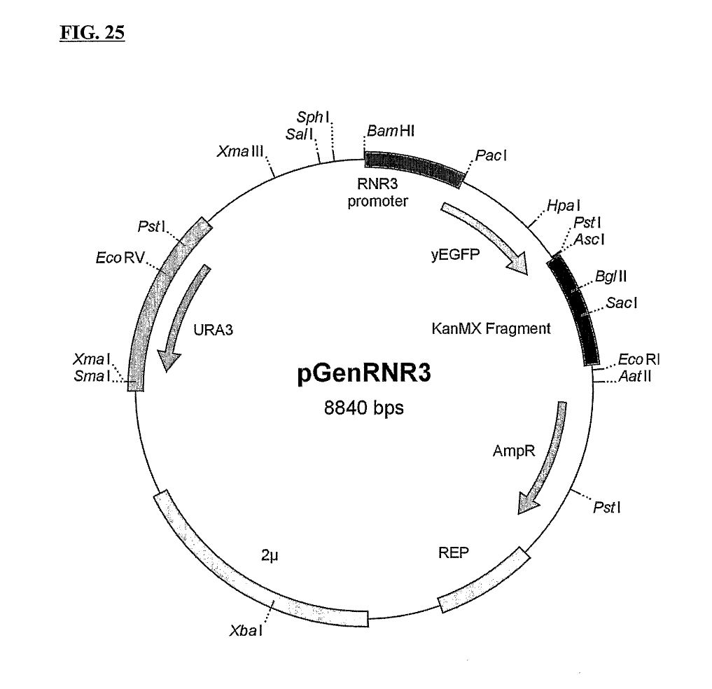

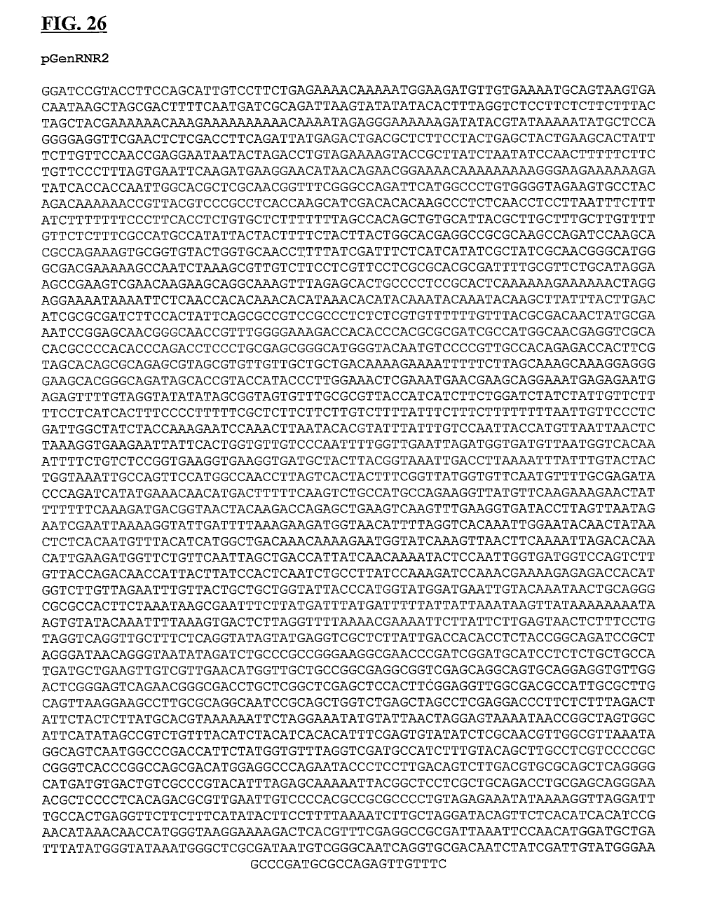

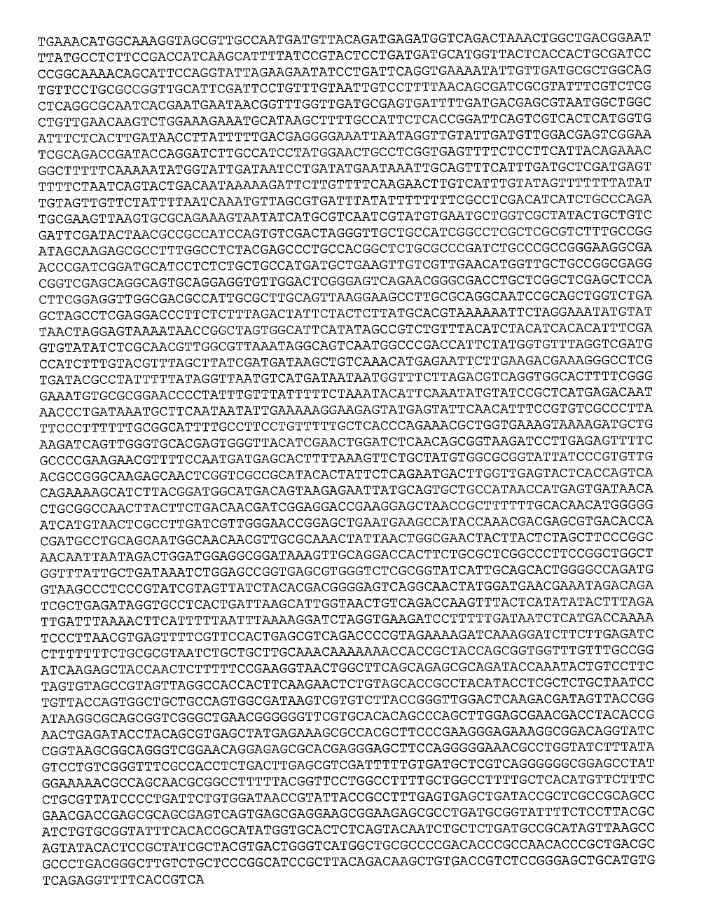

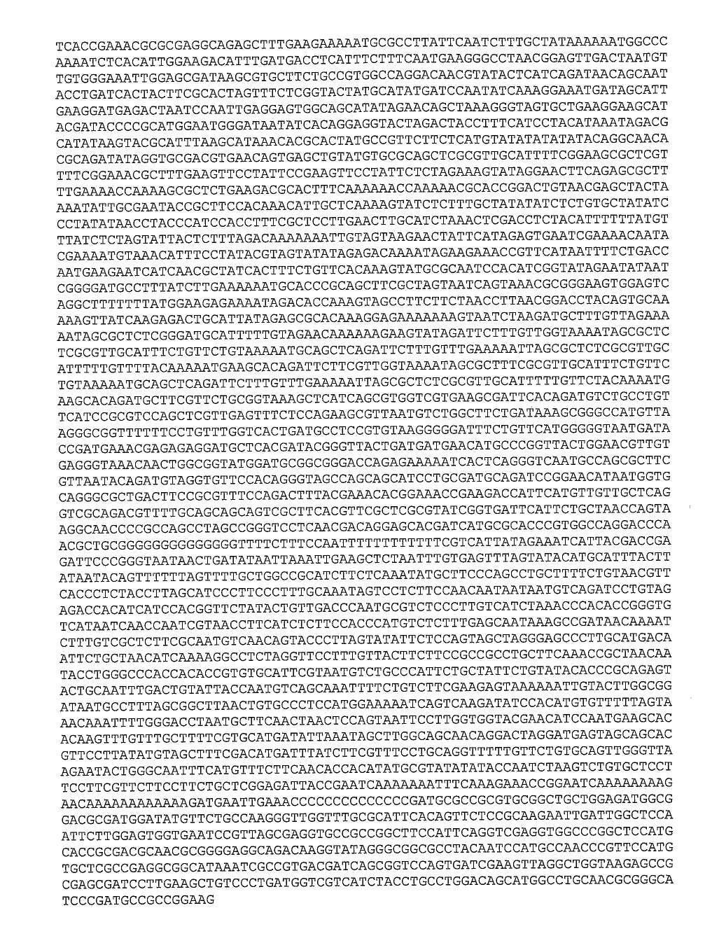

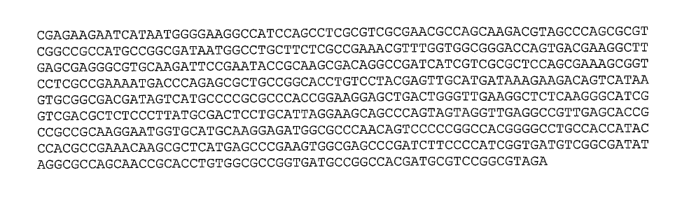

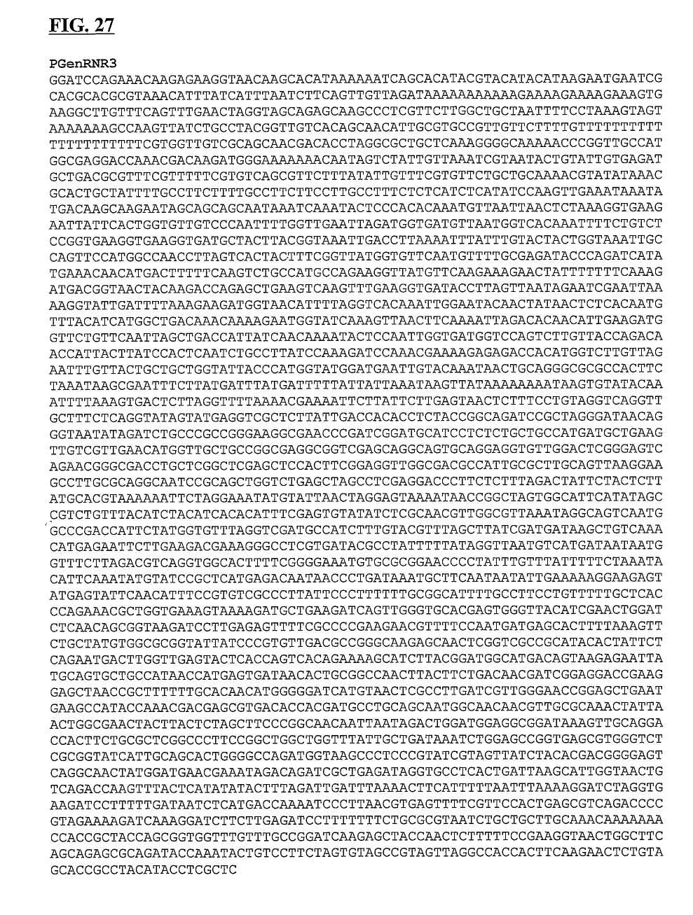

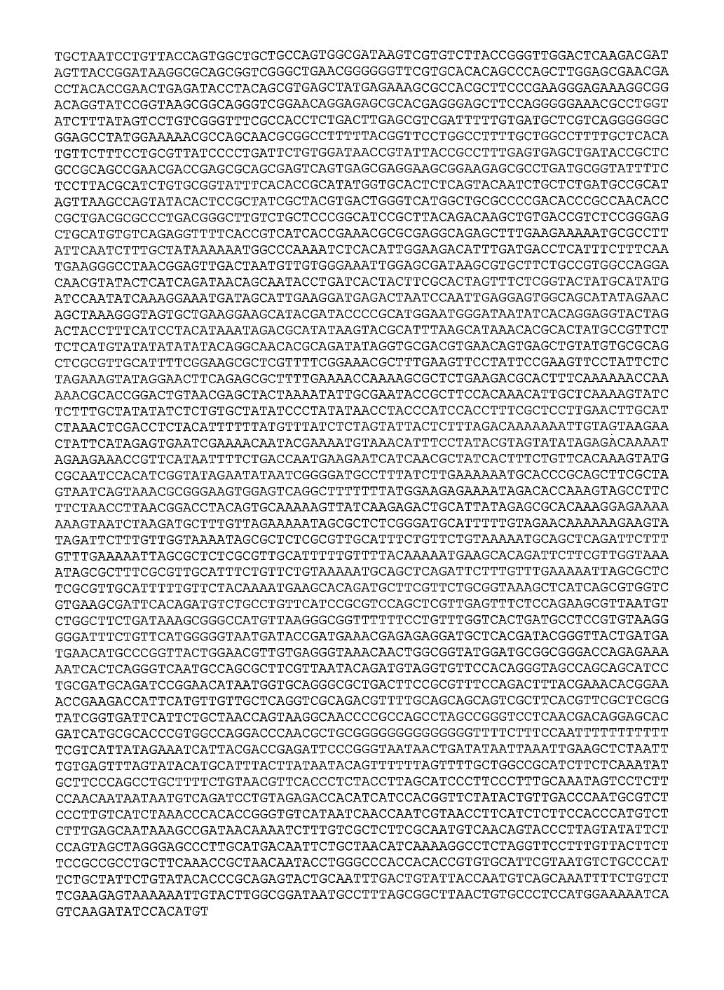

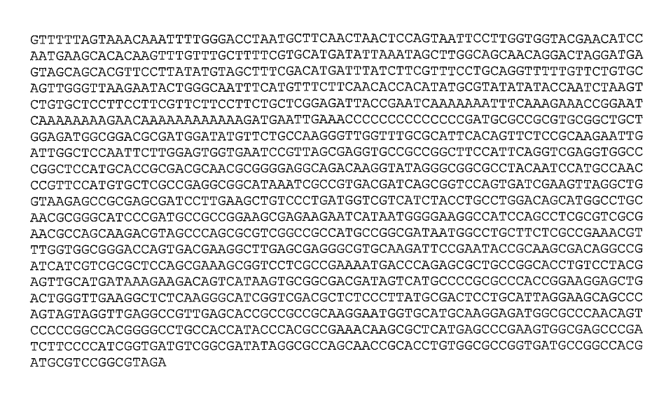

Most preferred recombinant DNA molecules comprise an RNR2 or an RNR3 regulatory element operatively linked to a DNA sequence that encodes a GFP or light emitting derivative thereof. Accordingly, a preferred recombinant vector according to the first aspect comprises the RNR2 or RNR3 gene operatively linked to GFP, or light emitting derivative thereof. It is especially preferred that the vector additionally comprises a non-functional kanMX3 module. Hence, a most preferred recombinant vector according to the first aspect of the invention is pGenRNR2, as illustrated in FIG. 24. The nucleotide sequence of pGenRNR2 is given in FIG. 26. Another preferred recombinant vector according to the first aspect of the invention is pGenRNR3, as illustrated in FIG. 25. The nucleotide sequence of pGenRNR3 is given in FIG. 27.

Therefore, most preferred recombinant vectors comprise RAD54, RNR2 or RNR3 regulatory element operatively linked to a DNA sequence encoding GFP or a light emitting derivative thereof, which preferably further comprises a non-functional kanMX3 module.

The recombinant vector according to the first aspect of the present invention may for example be a plasmid, cosmid or phage. Such recombinant vectors are of great utility when replicating the DNA molecule. Furthermore, recombinant vectors are highly useful for transforming cells with the DNA molecule and may also promote expression of the reporter protein.

The vectors according to the first aspect and as made using the method of the second aspect are particularly useful in the pharmaceutical industry for carrying out genotoxicity screens on novel compounds in the laboratory. In particular, each of the vectors is able to autonomously replicate in a host yeast cell due to the presence of a 2μ element. It will be appreciated that, due to legislation involving use of genetically modified organisms, it is especially preferred that the vectors are only used in an enclosed environment, and not released in to the environment.

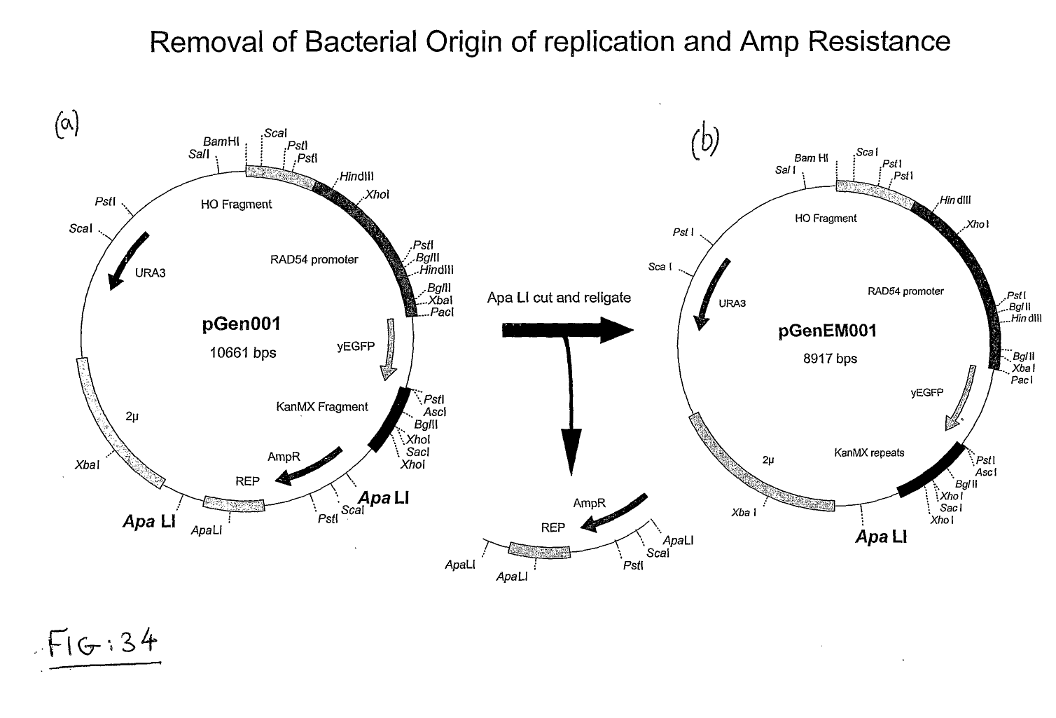

However, in some circumstances, it may be desirable to take advantage of the functional elements RAD54 and GFP of pGen001 (FIG. 15), RNR2 and GFP of pGenRNR2 (FIG. 24), and RNR3 and GFP or pGenRNR3 (FIG. 25), but in the environment instead of in the laboratory, i.e. it may be required to release the vectors in to the environment. Accordingly, the inventors have removed the bacterial origin of replication and also the gene conferring ampicillin resistance from each of these vectors. To illustrate this, FIG. 34 shows pGen001 (FIG. 34a) being digested with Apa L1 to produce the vector pGenEM001, and is shown in FIG. 34b. A similar digestion of pGenRNR2 or pGenRNR3 with Apa L1 (not shown) will also remove the bacterial origin of replication and ampicillin resistance gene.

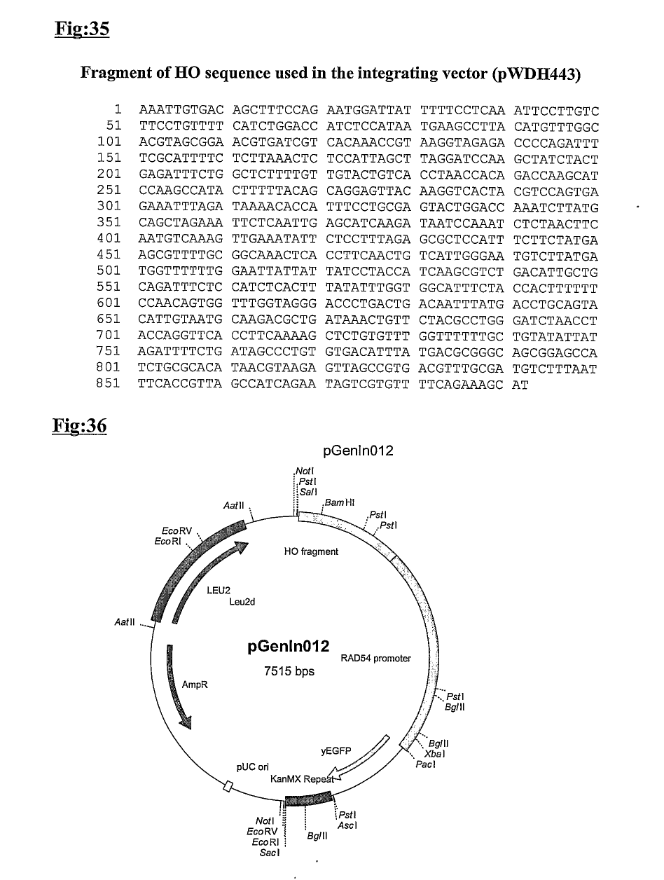

Instead of an autonomously replicating vector, the recombinant vector may be designed such that the vector and DNA molecule of the first aspect integrate into a chromosome of the host cell. Such integration has the advantage of improved stability compared to replicative plasmids. In this case, DNA sequences, which favour targeted integration (e.g. by homologous recombination) are desirable. For example, incorporation into the recombinant vector of fragments of the HO gene from chromosome IV of S. cerevisiae favours targeted integration in S. cerevisiae or cell lines derived therefrom. It is preferred that the fragment of the HO gene has the sequence as shown in FIG. 35, or a derivative thereof. It may also be possible to integrate multiple copies of the integrating vector into the genome of the host cell. This will allow greater expression, and increase the signal output of the light emitting reporter protein even further.

A preferred vector may therefore comprise the RAD54 gene or the RNR2 gene or the RNR3 gene, operatively linked to GFP, or a light emitting derivative thereof, a non-functional kanMX3 module, and a nucleotide sequence adapted to integrate into the genome of a target cell. The nucleotide sequence may be a fragment of the HO gene.

Using the polymerase chain reaction the LEU2-d gene (the Saccaromyces cerevisiae wild type LEU2 gene with a 29 bp truncated promoter) was amplified with flanking AatII cloning sites from the plasmid pEMBL-yex4 (Cesareni et al, 1987). This fragment was cloned into pWDH443 (Walmsley et al, 1997) to give the plasmid pGenIn011. This plasmid was further modified by removing 20.1 kb of the KanMX module by Sac I digestion and religation, resulting in a non-functional KanMX module. This plasmid was named pGenIn012. This plasmid was cut with Bam HI prior to transformation into the yeast strain FF18984 to facilitate plasmid integration at the HO locus.

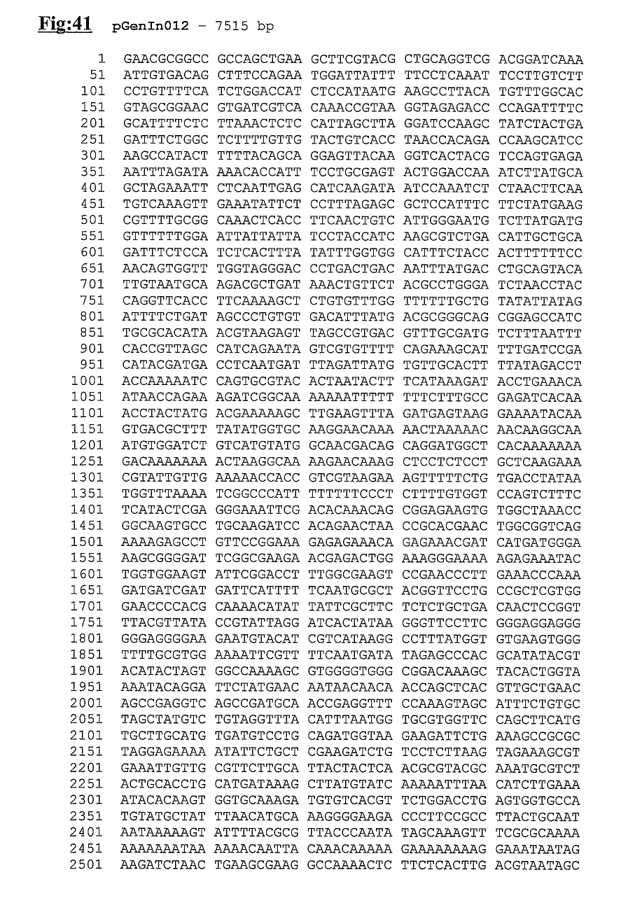

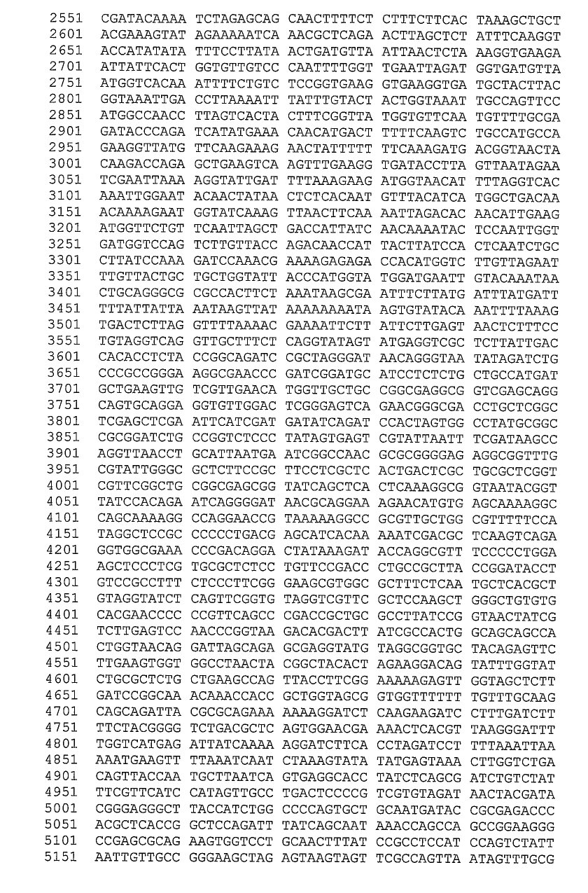

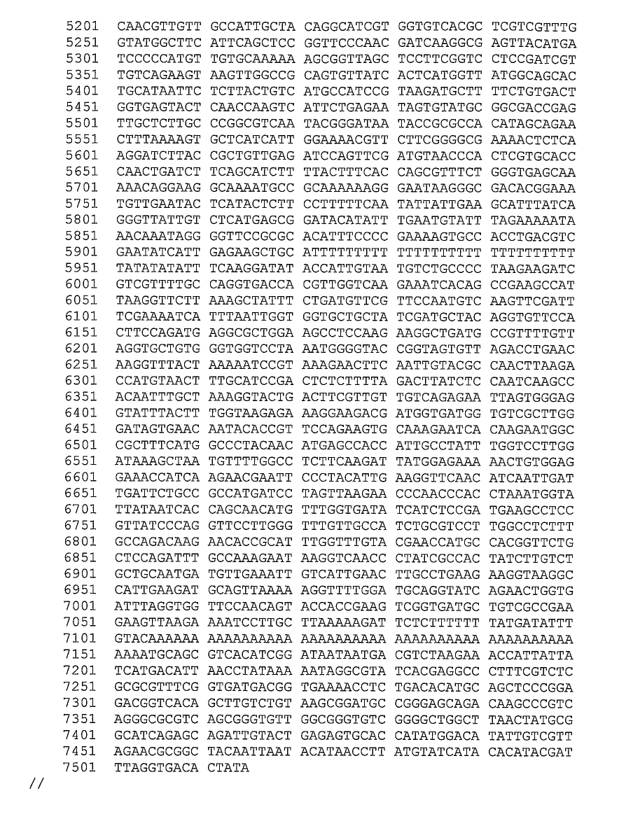

Hence, a preferred integrating vector according to the invention is referred to as pGenIn012, and is illustrated in FIG. 36. The full sequence of this vector is shown in FIG. 41, and is 7515 bp in length. pGenIn012 comprises RAD54 and it will be appreciated that similar integrating vectors comprising RNR2 or RNR3 although not illustrated are in accordance with the invention. pGenIn012 comprises a LEU2-d selectable marker and a non-functional kanMX3 module.

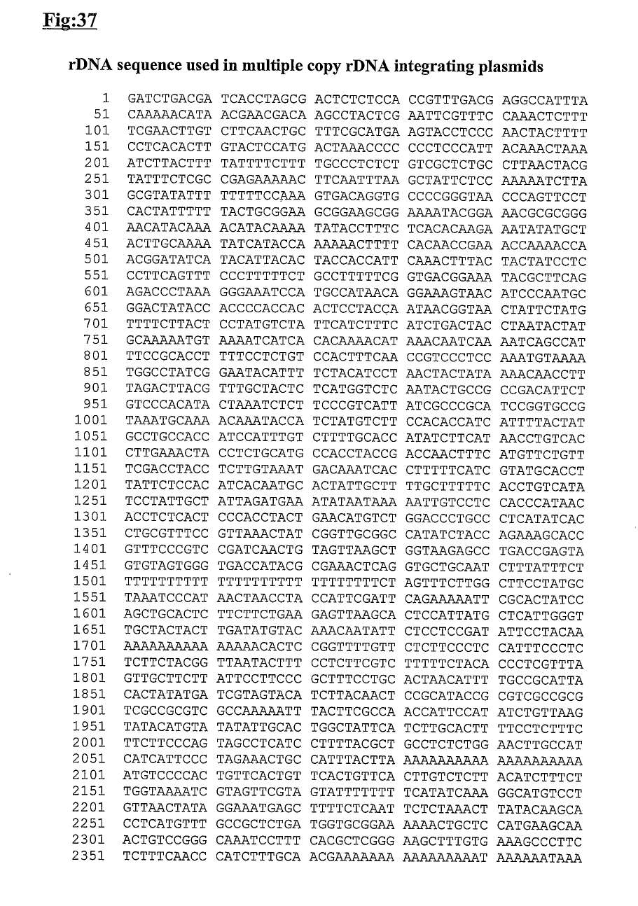

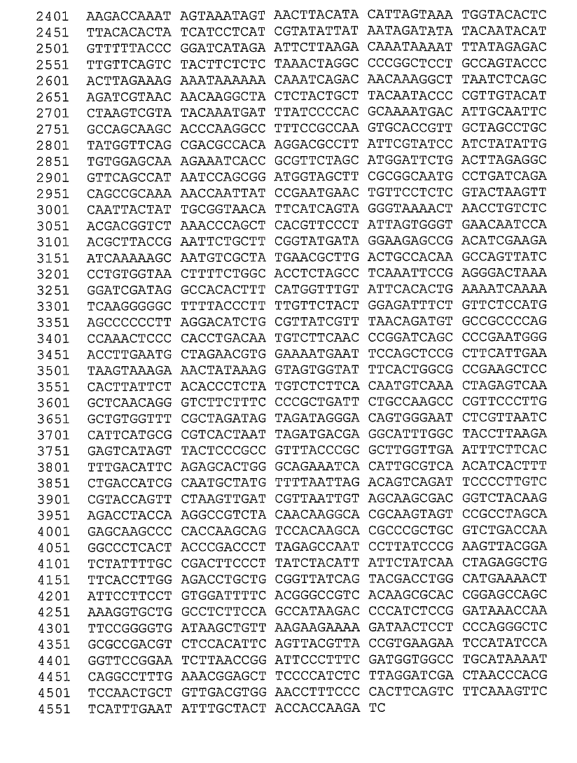

Other DNA sequences, which favour targeted integration into the genome, and which may be incorporated into the recombinant vector include sequences from the ribosomal DNA array of S. cerevisiae. Such rDNA sequences favour targeted integration in to chromosome XII of S. cerevisiae, or cell lines derived therefrom. It is preferred that the rDNA sequence has the sequence as shown in FIG. 37, or is a derivative thereof.

A 4.5 kb Bgl II cut rDNA fragment from the Saccaromyces cerevisiae genome was cloned into Bam HI cut pGenIn012 referred to above. As this fragment was cloned in two different orientations (which lead to different orientations of integration) the two different forms of these plasmids were named pGenIn022A and pGenIn022B. The plasmid, pGenIn022A, was cut with Sph I prior to transformation into the yeast strain FF18984 to facilitate plasmid integration within the tandem rDNA repeats of the Saccaromyces cerevisiae genome.

A preferred vector may therefore comprise the RAD54 gene or the RNR2 gene or the RNR3 gene, operatively linked to GFP, or a light emitting derivative thereof, a non-functional kanMX3 module, and a nucleotide sequence adapted to integrate into the genome of a target cell, wherein the nucleotide sequence may be rDNA sequence.

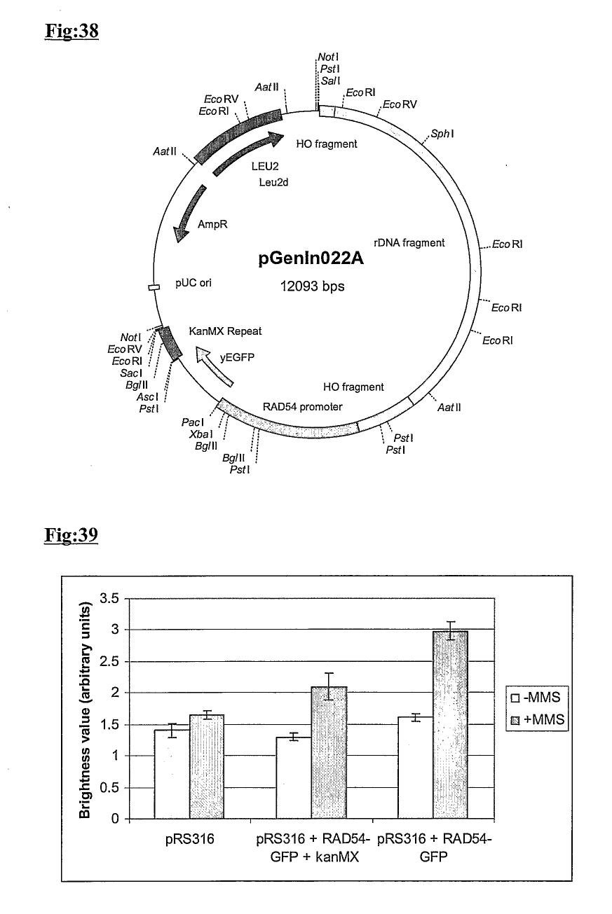

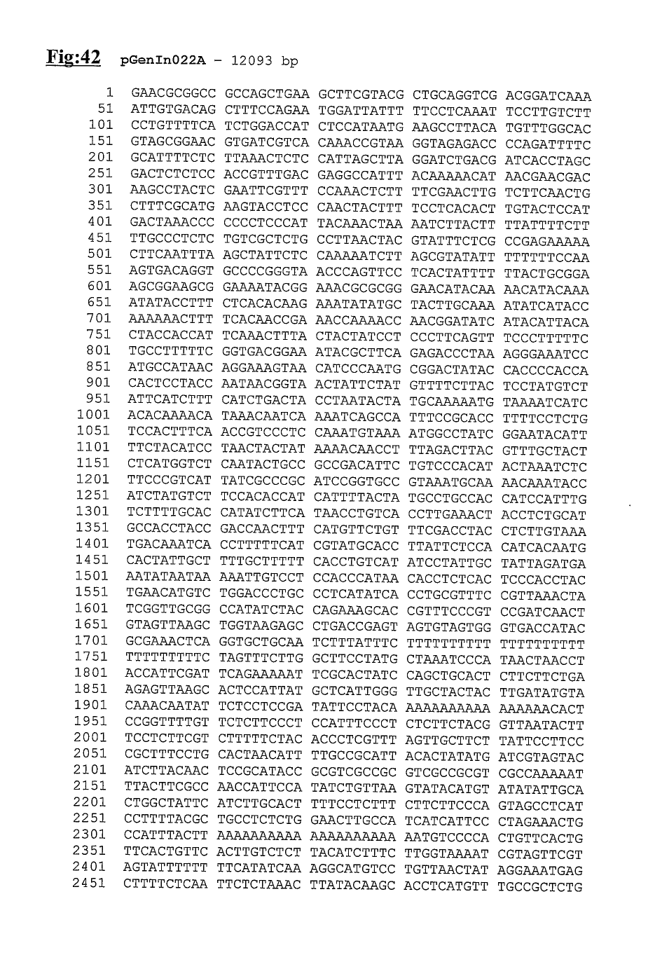

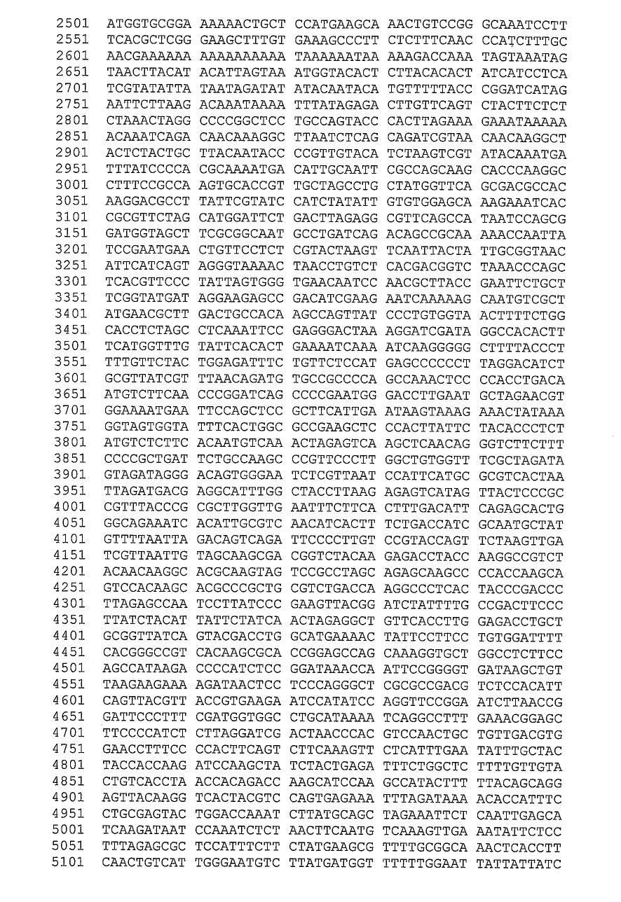

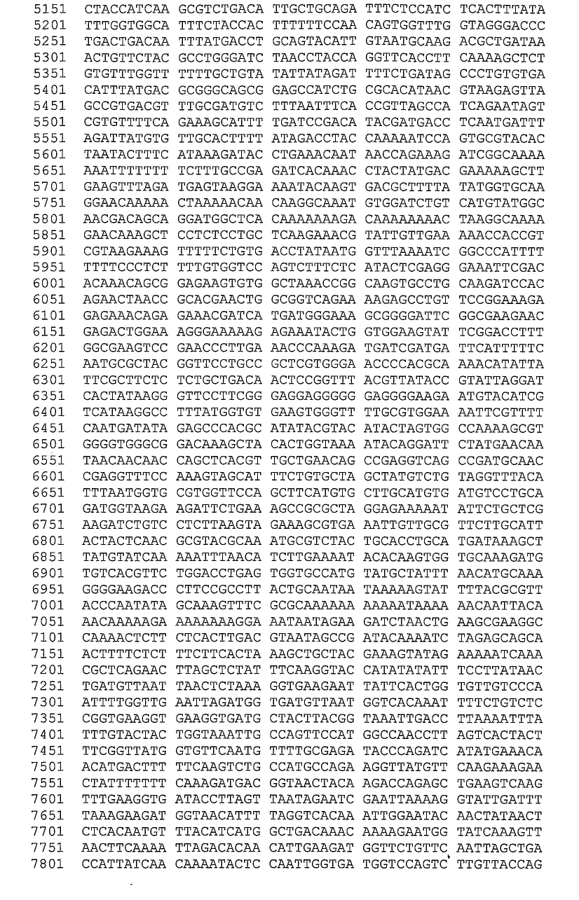

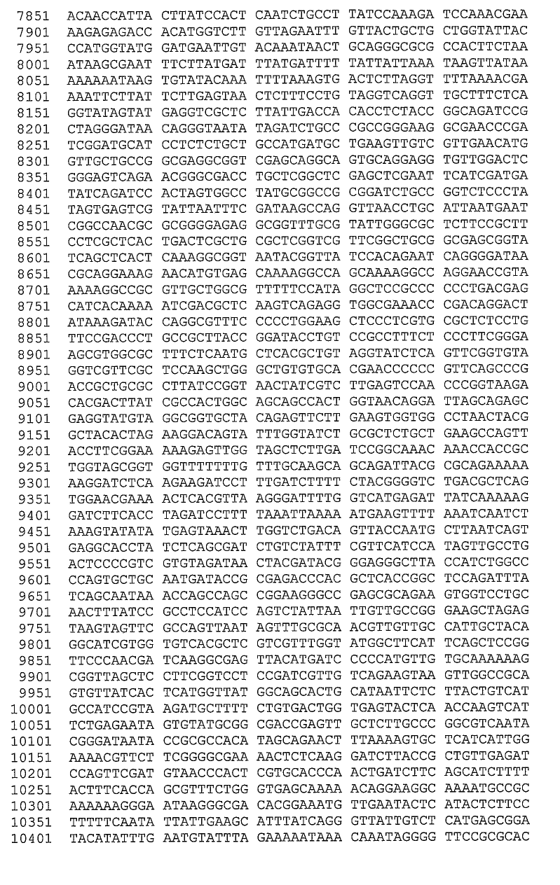

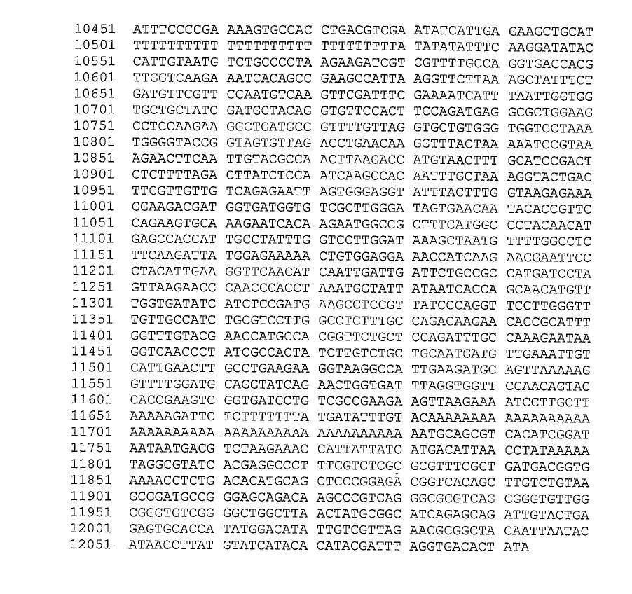

Hence, a preferred integrating vector according to the invention is referred to as pGenIn022 A-form, and is illustrated in FIG. 38. The full sequence of this vector is shown in FIG. 42, and is 12093 bp in length. pGenIn022 A-form comprises RAD54 and it will be appreciated that similar integrating vectors comprising RNR2 or RNR3 although not illustrated are in accordance with the invention. pGenIn022A comprises the LEU2-d selectable marker and a non-functional kanMX3 module.

FIG. 40 illustrates (i) the effect of the presence of kanMX on the brightness of pGenIn012, i.e. the integrating form of RAD54-GFP-HO (all without MMS) when integrated into the chromosome at HO (HO int); and (ii) the effect of the presence of kanMX on the brightness of pGenIn022A, i.e. the integrating form of RAD54-GFP-rDNA. It should be appreciated that rDNA B int and rDNA A int-A and B refer to orientation of the reporter cassette in the chromosome. It is evident that rDNA A int-A is the brightest construct, possibly due to effects of the surrounding DNA sequence and protein interactions therewith, such as steric hindrance.

Preferably recombinant vectors may be formed from pFA vectors or derivatives thereof, which are known to the art (see Wach et al. (1994) Yeast 10 p 1793-1808).

Preferred recombinant vectors are derived from yEGFP-444 disclosed in WO 97/44149 (the disclosure of which is incorporated herein by reference). yEGFP-444 is also known as pWDH445.

According to the third aspect of the invention the recombinant vector is incorporated within a cell. Such host cells may be prokaryotic or eukaryotic. Suitable host cells include bacteria, plant, yeasts, insect and mammalian cells. Preferred host cells are yeast cells such as Saccharomyces cerevisiae. Yeast are preferred because they can be easily manipulated like bacteria but are eukaryotic and therefore have DNA repair systems that are more closely related to humans than those of bacteria. Another benefit of using yeast cells as a host is that DNA repair systems are inducible in yeast unlike in humans where the repair systems are largely constitutive.

Preferred yeast cells include:

(i) Y485 in haploid form;

(ii) Y486 (also known as FF18984) in haploid form;

(iii) Y485/486 in diploid form;

(iv) FY73

(v) YLR030w.alpha.; and

(vi) Y300.

These strains may all be found in national yeast strain collections.

We have found that (i), (ii) and (iii) above are particularly useful strains for use according to the method of the invention. A preferred strain comprises GenT01. The reporter strain (GenT01) comprises the yeast FF18984 containing a replicative plasmid (pGen001 shown in FIG. 15) containing the entire upstream non-coding DNA sequence of the RAD54 gene fused to the yeast-enhanced Aequorea victoria GFP gene.

Host cells used for expression of the protein encoded by the DNA molecule are ideally stably transformed, although the use of unstably transformed (transient) cells is not precluded.

Transformed cells according to the third aspect of the invention may be formed by following procedures described in the Example. The cell is ideally a yeast cell (for instance one of the strains described above). Such transformed cells may be used according to the method of the fourth aspect of the invention to assess whether or not agents induce or potentiate DNA damage. GFP expression is induced in response to DNA damage and the light emitted by GFP may be easily measured using a fluorimeter as an index of the DNA damage caused. For instance, the light emitted by GFP at 511 nm (after excitation between 475 and 495 nm—e.g. 488 nm) in response to DNA damage, may be evaluated either in a suspension of a defined number of whole cells or from a defined amount of material released from cells following breakage. Alternatively, light emitted by GFP at 520 nm may be evaluated through a 535 nm filter.

The method of the fourth aspect of the invention is particularly useful for detecting agents that induce DNA damage at low concentrations. The methods may be used to screen compounds, such as candidate medicaments, food additives or cosmetics, to assess whether it is safe to expose a living organism, particularly people, to such compounds.

Alternatively the method of the fourth aspect of the invention may be employed to detect whether or not water supplies are contaminated by DNA damaging agents or agents that potentiate DNA damage. For instance, the methods may be used to monitor industrial effluents for the presence of pollutants that may lead to increased DNA damage in people or other organisms exposed to the pollution.

When the methods are used to detect whether or not water supplies are contaminated, the cells according to the second aspect of the invention are ideally unicellular organisms such as bacteria, algae, protozoa and particularly yeast.

The expression of light emitting reporter protein may be monitored according to the method of the invention from cell extracts or from samples containing intact, whole cells.

There are several advantages associated with the use of whole cells. As there is no requirement to break open cells, the number of treatment steps is reduced. The production of extracts requires cell-harvesting, washing, breakage with glass beads and centrifugation to clear the extract. The reduction in treatment steps also reduces the risk of errors arising in handling and makes the method much faster. Furthermore, cell density and light emission can be made simultaneously giving greater sensitivity. Therefore, the method of the invention is preferably performed by growing cells transformed with a recombinant vector according to the first aspect of the invention (such as pGen001, pGenRNR2, pGenRNR3, pGenEM001, pGenIn012, or pGenIn022A), incubating the cells with the agent which putatively causes DNA damage for a predetermined time and monitoring the expression of the light emitting reporter protein directly from a sample of the cells.

When whole cells are used they are preferably contained in low fluorescence growth medium. This can obviate the need to wash the cells before measurements are made and therefore reduce the number of steps in the method further. For instance, preferred yeast according to the third aspect of the invention may be grown in F1 medium (described in Walmsley et al. (1983) Mol. Gen. Genet. 192 p 361-365 and the Example).

A preferred method of fluorescence detection is described in U.S. Pat. No. 6,509,161. This method is particularly useful when the light emitting report is GFP or a derivative thereof.

A preferred method of testing for DNA damage according to the fourth aspect of the invention comprises the steps of: (1) preparing a microplate for use in an assay; (2) conducting the assay in the microplates; (3) collecting and analysing the data; and (4) making a judgment on DNA damage and the consequences.

Details of steps (1)-(4) for conducting a most preferred test for DNA damage are given below.

### (1) Microplate Preparation.

Assays were carried out in 96 well, black, clear-bottomed microplates. For example Matrix ScreenMates, Cat. No. 4929, Apogent Discoveries, USA, or Corning (BV, Netherlands: Cat. No. 3651). A number of alternative microplates were assessed, though the variable background absorbance and fluorescence both within and between plates from individual manufacturers were generally unacceptable, leading to the currently preferred choice. It will therefore be appreciated that microplates used according to the invention preferably have consistent absorbance and fluorescence between plates and batches thereof.

The assay plates can be filled using a liquid handling robot. For example the MicroLabS single probe, from Hamilton GB Ltd., Birmingham or a Genesis 8-probe robot (Tecan UK Ltd. Theale. UK). Microplates can also be filled rapidly and effectively using a multi-channel pipette.

### (2) Assay

The following standard protocol may be followed. A 1 mM stock of a test chemical, or sample containing an agent that putatively caused DNA damage, was prepared in 2% v/v aqueous DMSO, and used to make 2 identical dilution series across a 96 well microplate and a ‘control’ (see below). To achieve this, 150 microlitres of the test chemical solution were put into 2 microplate wells. Each sample was serially diluted by transferring 75 microlitres into 75 microlitres of 4% DMSO, mixing, and then taking 75 microlitres out and into the next well. This produced 9 serial dilutions of 75 microlitres each.

Controls were added as follows:
a. Test compound/sample containing agent alone, to provide information on compound absorbance/fluorescence
b. Yeast cultures diluted with 4% DMSO alone, to give a measure of maximum proliferative potential
c. MMS as a genotoxicity control: ‘high’=0.00125% v/v, ‘low’=0.0001875% v/v
d. Methanol as a cytotoxicity control: ‘high’=3.5% v/v, ‘low’=1.5% v/v.
e. Growth medium alone, to confirm sterility/lack of contamination

Stationary phase cultures of yeast cells according to the invention (e.g. GenT0) and also a control yeast having a non-functional reporter (e.g. GenC01), were diluted to an optical density (OD600 nm)=0.2 in double strength F1 medium (Billinton et al., 1998). 75 microlitres of the yeast suspension were added to each well of the diluted chemical: GenT01 to one series and GenC01 to the second, and to appropriate standards and controls. After the plates were filled, they were sealed using either a gas permeable membrane (for example Breath-easy, Diversified Biotech, USA) or a plastic lid, and then incubated without shaking, overnight at 25° C.

### (3) Data Collection and Handling.

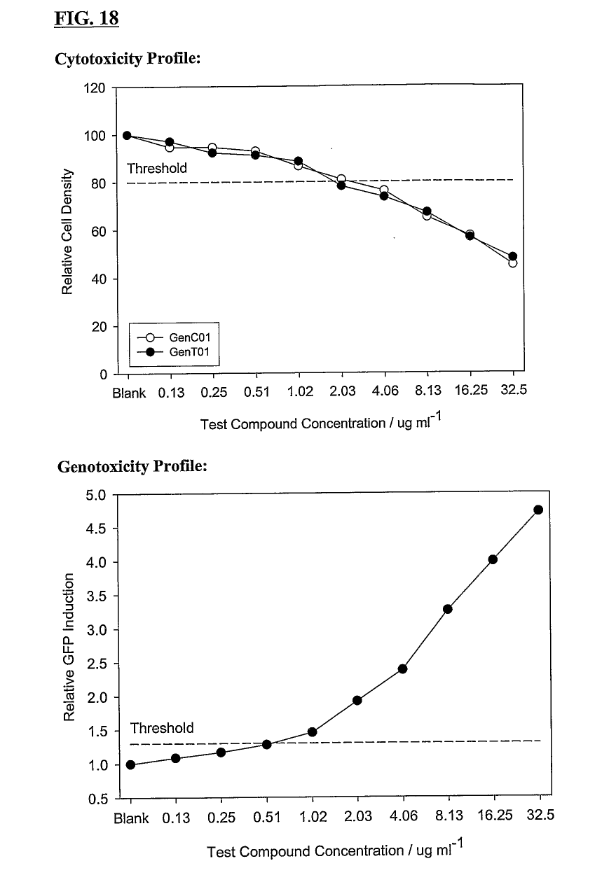

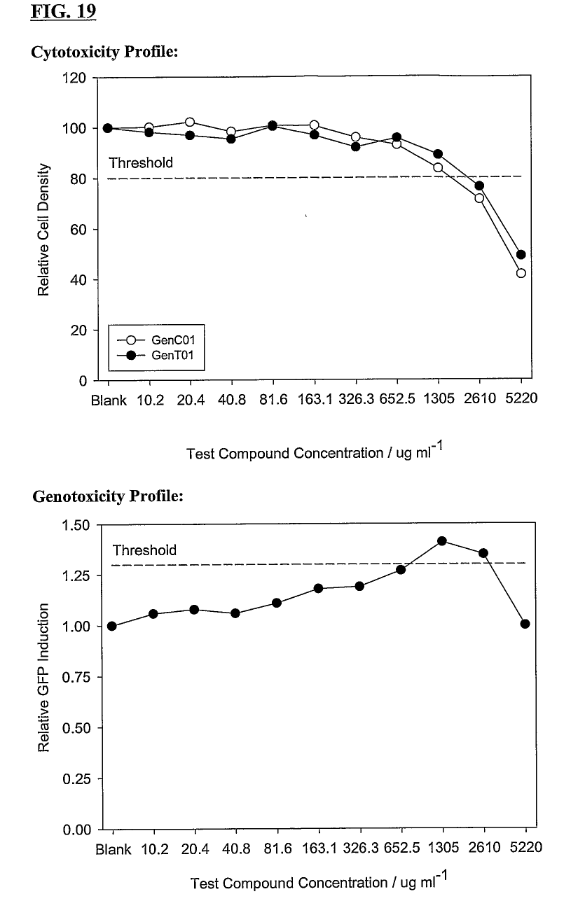

Following overnight incubation, fluorescence and absorbance data were collected from the microplates. Two different microplate readers which combine fluorescence and absorbance functionality have been used, and comparable data were obtained. These were a Tecan Ultra-384 (Tecan UK Ltd.): excitation 485 nm/emission 535 nm with an additional dichroic mirror (reflectance 320 nm-500 nm, transmission 520 nm-800 μm), and a BMG PolarStar (BMG Labtechnologies, Germany): excitation 485 μm/emission 520 nm. Absorbance was measured through a 620 nm filter in both instruments. The data were transported into a Microsoft Excel spreadsheet, and converted to graphical data (see typical data in FIGS. 18 and 19 for Example 2). Data processing is minimal: absorbance data give an indication of reduction in proliferative potential and these data were normalised to the untreated control (=100% growth). Fluorescence data were divided by absorbance data to give ‘brightness units’, the measure of average GFP induction per cell. These data were normalised to the untreated control (=1). In this way, one can distinguish between a small number of highly fluorescent cells and a large number of weakly fluorescent cells. In order to correct for induced cellular autofluorescence and intrinsic compound fluorescence, the brightness values for the GenC01 strain were subtracted from those of GenT01. This makes visual assessment of the data more reliable. All the data were checked with and without this correction, and the decision (see below), on whether or not a compound was classified as being genotoxic, was not affected.

### (4) Decision Thresholds.

It is useful to have clear definitions of positive and negative results from routine assays and such definitions have been derived, taking into account the maximum noise in the system and data from chemicals where there is a clear consensus on genotoxicity and mechanism of action. Naturally it is also possible for users to inspect the numerical and graphical data and draw their own conclusions. For example an upward trend in genotoxicity data that did not cross the threshold might still distinguish two compounds. The decision thresholds were set as follows:

The cytotoxicity threshold is set at 80% of the cell density reached by the untreated control cells. This is greater than 3 times the standard deviation of the background. A positive cytotoxicity result (+) is concluded if 1 or 2 compound dilutions produce a final cell density lower than the 80% threshold. A strong positive cytotoxicity result positive (++) is concluded when either (i) three or more compound dilutions produce a final cell density lower than the 80% threshold or (ii) at least one compound dilution produces a final cell density lower than a 50% threshold. A negative result (−) is concluded when no compound dilutions produce a final cell density lower than the 80% threshold. The lowest effective concentration (LEC) is the lowest test compound concentration that produces a final cell density below the 80% threshold.

The compound absorbance control allows a warning to be generated if a test compound is significantly absorbing. If the ratio of the absorbance of the compound control well to a well filled with diluent alone is >2, there is a risk of interference with interpretation. The cytotoxicity controls indicate that the yeast is behaving normally. The ‘high’ methanol standard should reduce the final cell density to below the 80% threshold, and should be a lower value than the ‘low’ standard.

The genotoxic threshold is set at a relative GFP induction of 1.3 (i.e. a 30% increase). This is greater than 3 times the standard deviation of the background. A positive genotoxicity result (+) is concluded if 1 or 2 compound dilutions produce a relative GFP induction greater than the 1.3 threshold. A strong positive genotoxicity result (++) is concluded if either (i) three or more compound dilutions produce a relative GFP induction greater than the 1.3 threshold or (ii) at least one compound dilution produces a relative GFP induction greater than a 1.6 threshold. A negative genotoxicity result (−) is concluded where no compound dilutions produce a relative GFP induction greater than the 1.3 threshold. The LEC is the lowest test compound concentration that produces a relative GFP induction greater than the 1.3 threshold. The genotoxic controls demonstrate that the strains are responding normally to DNA damage. The ‘high’ MMS standard must produce a fluorescence induction >2, and be a greater value than the ‘low’ MMS standard. Anomalous brightness data is generated when the toxicity leads to a final cell density less than 30% that of the blank. Genotoxicity data is not calculated above this toxicity threshold. Compounds that tested negative for genotoxicity, were re-tested up to 10 mM, or to the limit of solubility or cytotoxicity.

The compound fluorescence control allows a warning to be generated when a compound is highly auto-fluorescent. If the ratio of the fluorescence of the compound control well to a diluent filled well is >5, there is a risk of interference with interpretation. In these cases (four in this study), fluorescence polarisation can be used to distinguish GFP from other sources of fluorescence (Knight et al., 2000, 2002). Both the Tecan and BMG instruments have this facility. Occasionally, compounds though not fluorescent themselves, induce cellular auto-fluorescence. This is apparent from rising brightness in the control (GenC01) strain in the absence of fluorescence from the chemical-only control. The routine subtraction of GenC01 from GenT01 data removes this interference from the data.

According to a preferred embodiment of the method of the fourth aspect of the invention FF18984 cells may be transformed with pGen001 and grown in F1 medium. According to another preferred embodiment of the method of the invention, FF18984 cells may be transformed with pGenRNR2 or pGenRNR3 and grown in F1 medium. Additionally, FF18984 may be transformed with any of pGenEM001, pGenin012, or pGenIn022A, and grown in F1 medium.

A putative DNA damaging agent (e.g. a food additive or potential medicament or an agent contained within a water sample or effluent sample) may then be added to the F1 medium containing the cells. The cells are then allowed to grow for a defined period of time after which a sample of the cells is removed and fluorescence measured therefrom. This measurement may be effected by estimating the cell concentration and fluorescence in the sample using nephelometry (light scattering). For example, cells can be illuminated at 600 nm and the scattered light (at 600 nm) estimated at 90 degrees to the incident beam. The light emitted by GFP can be measured by excitation at 475-495 nm (e.g. 487 nm) and fluorescent light emitted at 518 nm measured at 90 degrees to the incident beam. Both measurements may be made in a single cuvette. A normalised GFP light emission is calculated by dividing the GFP fluorescence value by the whole cell light scattering value (at 600 mm). This embodiment of the invention has the advantage that it may be easily carried out with a minimum of steps (i.e. an incubation period followed by direct fluorescence measurement).

The method of the invention should ideally employ sensitive fluorimeters and reduce light scattering in order that light emission can be accurately measured from the reporter protein. We have found that sensitivity can be improved by using a 487 nm filter which is introduced between the sample chamber and the emission-detector of the fluorimeter. Such a filter further reduces the impact of light scattering and improves the sensitivity of the method when samples containing whole cells are used.

Preferred methods of fluorescence detection and quantitation are given in the Examples

According to a further aspect of the invention, there is provided a recombinant vector comprising a recombinant DNA molecule comprising a regulatory element that activates gene expression in response to DNA damage operatively linked to a DNA sequence that encodes a light emitting reporter protein and a DNA vector characterised in that the vector comprises an origin of replication; at least one selectable marker; and when used to transform a cell, does not alter the sensitivity of the cell to geneticin.

According to a further aspect of the invention, there is provided a recombinant vector comprising a recombinant DNA molecule comprising an RNR regulatory element operatively linked to a DNA sequence that encodes a light emitting reporter protein and a DNA vector characterised in that the vector comprises an origin of replication; at least one selectable marker; and when used to transform a cell, does not alter the sensitivity of the cell to geneticin.

All of the features described herein (including any accompanying claims, abstract and drawings), and/or all of the steps of any method or process so disclosed, may be combined with any of the above aspects in any combination, except combinations where at least some of such features and/or steps are mutually exclusive.

The present invention will now be described, by way of examples, with reference to the accompanying drawings in which:

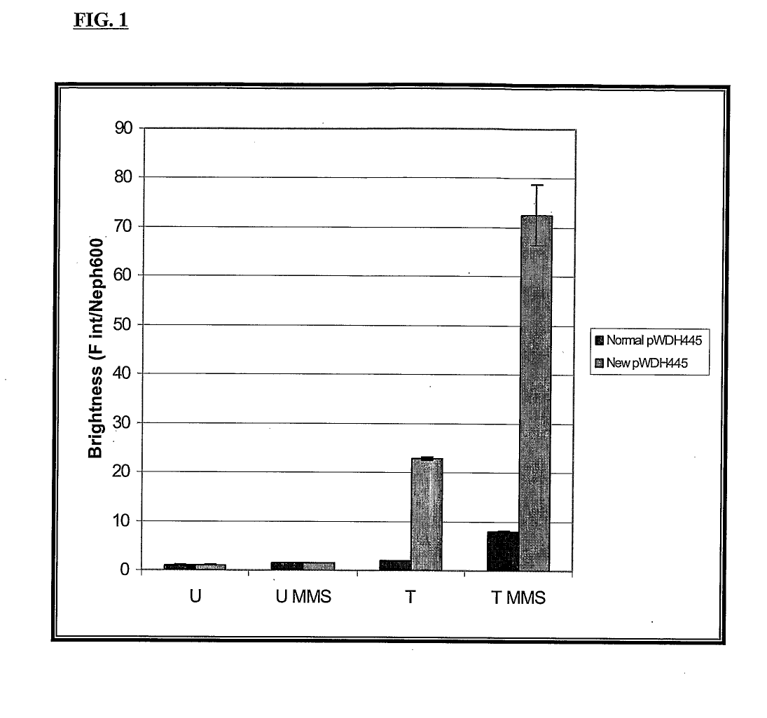

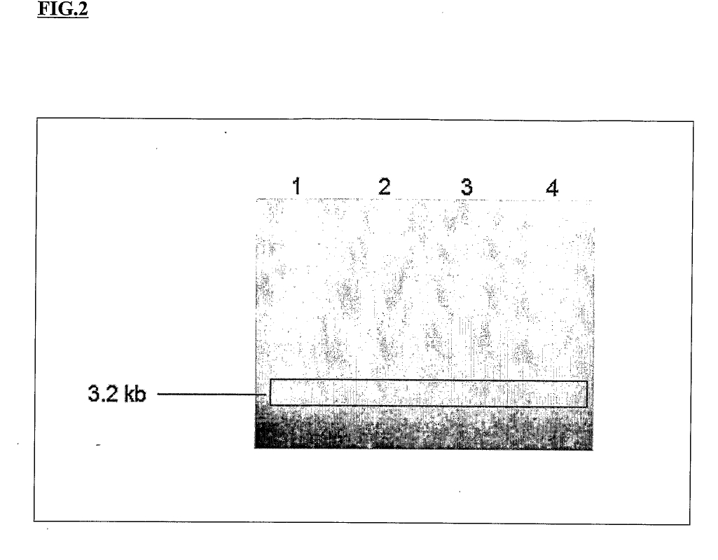

FIG. 1 shows the difference between the brightness values obtained from FF18984 cells transformed with pWDH445, exhibiting normal levels of brightness (▪) and enhanced brightness (▪) without and with exposure to MMS. U, untransformed FF18984 cells not treated with MMS; U MMS, untransformed cells exposed to 0.005% MMS (15 hours); T, cells transformed with pWDH445 but not treated with MMS; T MMS, cells transformed with pWDH445 and exposed to 0.005% MMS; Brightness, fluorescence intensity (F int) corrected for culture density using the intensity of scattered light at 600 nm (Neph600). Each bar represents the average brightness value of three independent cultures. Both uninduced and MMS-induced brightness values are clearly significantly enhanced in the new transformants relative to the original transformants.

FIG. 2 shows an ethidium bromide stained agarose gel of pWDH445 cut with BamH I and Asc I. The lanes contain the following: Lane 1, pWDH445; Lane 2, plasmid isolated from an enhanced brightness transformant; Lane 3, pWDH445 isolated from a normal brightness yeast transformant; and Lane 4, a second isolate from a transformant exhibiting enhanced brightness. BamH I-Asc I restriction of pWDH445 liberates a fragment of 3.2 kb (boxed) bearing HO-RAD54promoter-yEGFP cassette. A band representing a 3.2 kb-sized fragment is visible in all four lanes.

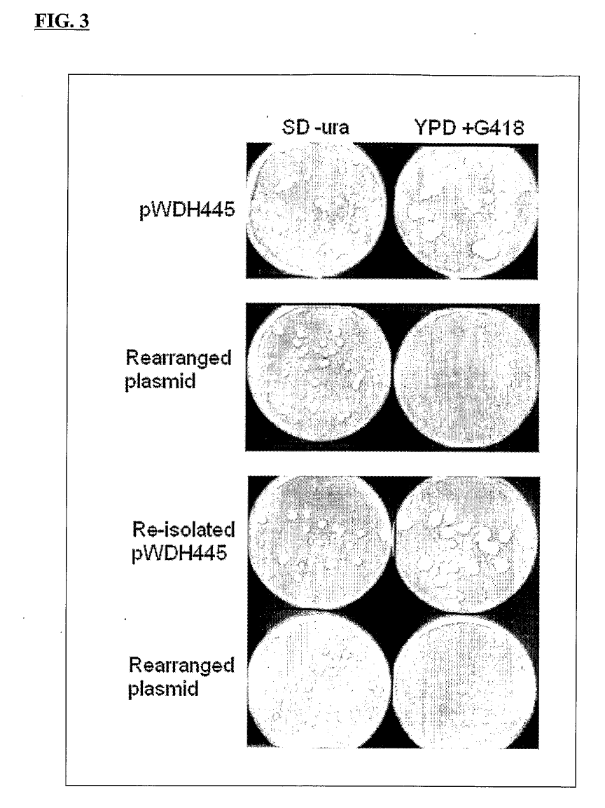

FIG. 3 shows FF18984 cells transformed with pWDH445 and cells re-transformed with re-isolated pWDH445 or rearranged plasmid (two independent isolates) isolated from transformants of enhanced brightness. The four sets of transformants were replica-plated onto SD medium deficient in uracil and YPD medium incorporating 200 μgml⁻¹ geneticin (G418) and incubated at 30° C. for 3 days. All four sets of transformants grew on SD-ura but only the cells bearing pWDH445 and re-isolated pWDH445 grew on YPD+G418.

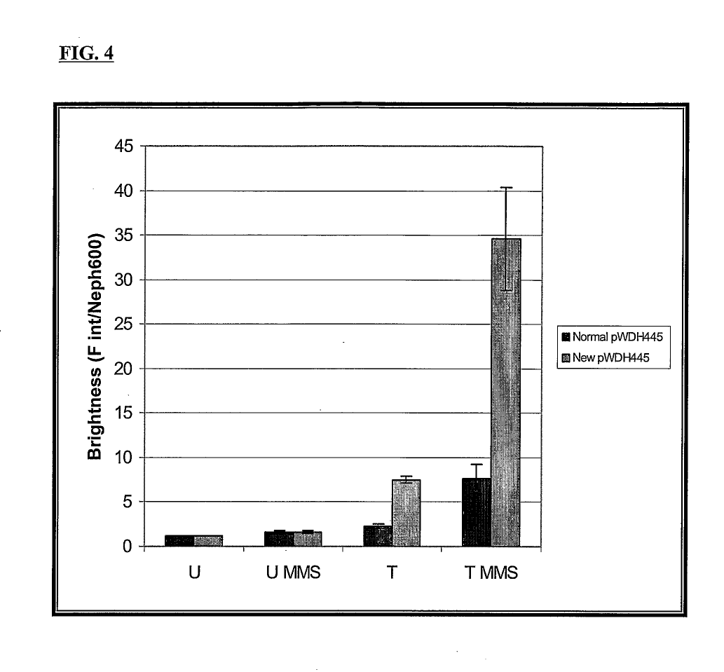

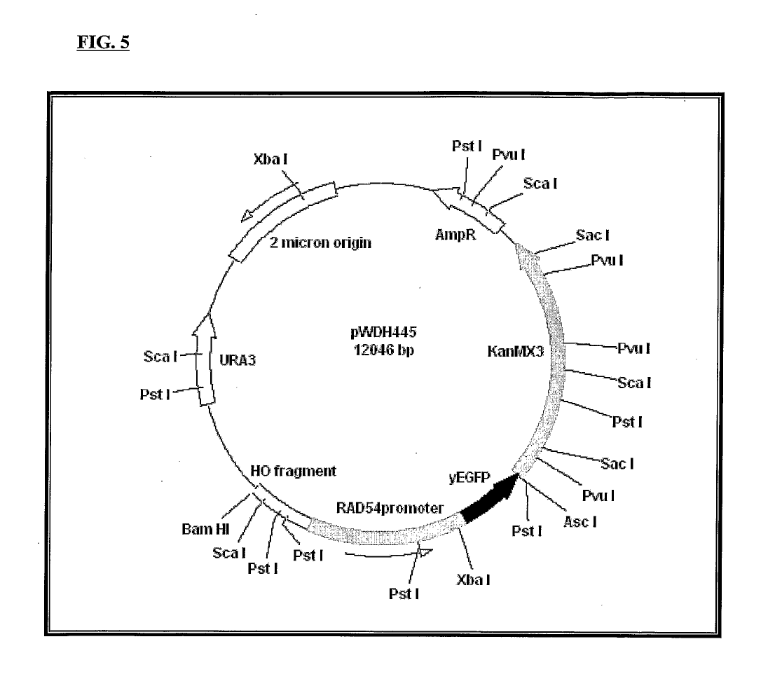

FIG. 4 demonstrates that FF18984 cells re-transformed with rearranged plasmid (▪) are brighter than FF18984 cells bearing pWDH445 (▪) without and with exposure to 0.005% MMS. U, untransformed FF18984 cells not exposed to MMS; U MMS, untransformed cells exposed to 0.005% MMS (15 hours); T, cells transformed with pWDH445 or rearranged plasmid not exposed to MMS; T MMS, cells transformed with pWDH445 or rearranged plasmid and exposed to 0.005% MMS; Brightness, fluorescence intensity (F int) corrected for culture density using the intensity of scattered light at 600 nm (Neph600). Each bar represents the average brightness value of six independent cultures. Cells re-transformed with the re-isolated rearranged plasmid are still brighter than normal pWDH445 transformants both without and with exposure to MMS.

FIG. 5 shows the restriction map of pWDH445 created for and used in this study. Base-pair sequences of the plasmid constituents were analysed for restriction enzyme cleavage sites using software available on the Stanford Genome Database (SGD) web-site. Major plasmid components are shown as blocked sections with the plasmid backbone (mainly bacterial sequences, such as the origin of replication) drawn represented by a line (−). The RAD54 promoter (▪)-yEGFP (▪) cassette and the kanMX3 cassette (▪) are shaded to highlight the main regions of interest, whilst Amp^{r} and URA3 are represented by open block arrows. The arrows represent the direction of transcription for individual components.

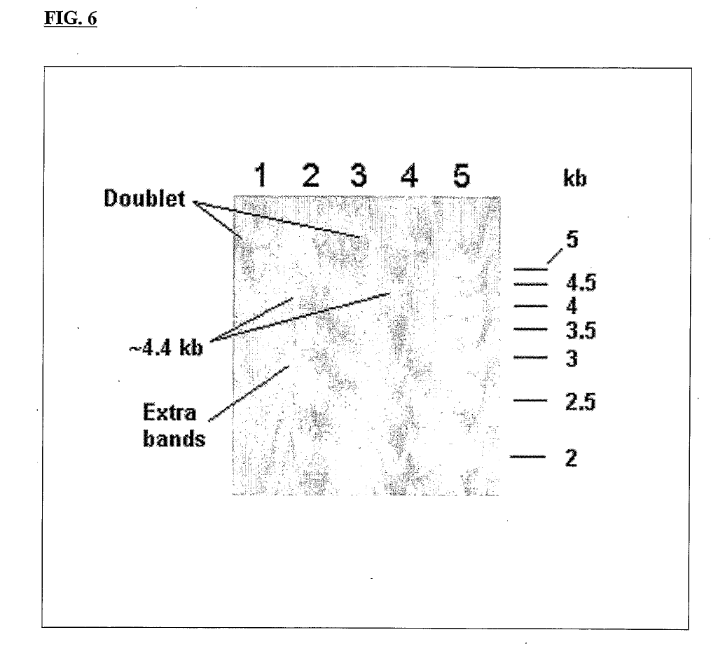

FIG. 6 shows an ethidium bromide stained agarose gel of the four plasmids (pWDH445, re-isolated pWDH445, and two rearranged plasmids) digested with Xba I. The lanes were loaded as follows: Lane 1, pWDH445; Lane 2, rearranged plasmid; Lane 3, re-isolated pWDH445; Lane 4, second rearranged plasmid; Lane 5, 500 bp ladder (5-0.5 kb) marker DNA. Xba I restriction of both rearranged plasmids produced a novel band representing a fragment of 4.4 kb, which was not detected for pWDH445 and re-isolated pWDH445. The rearranged plasmids also appear to be missing one band of a 6 kb doublet observed for the pWDH445 digestions. Extra bands in lane 2 reveal that one rearranged plasmid is larger than pWDH445, while the loss of a band from the doublet (and no extra bands) suggest that the rearranged plasmid loaded in lane 4 is smaller than pWDH445.

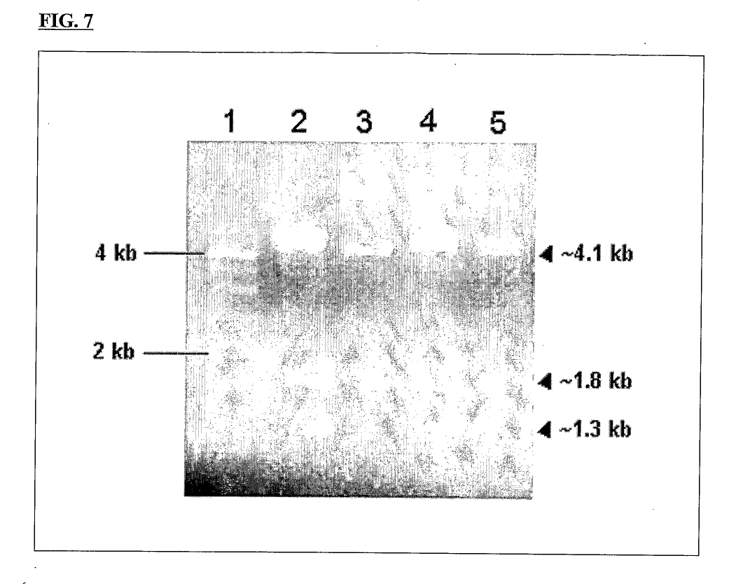

FIG. 7 shows an ethidium bromide stained agarose gel of the four plasmids (pWDH445, re-isolated pWDH445, and two rearranged plasmids) restriction digested with Sca I. The lanes were loaded as follows: Lane 1, 500 bp ladder (5-0.5 kb) molecular weight marker DNA; Lane 2, pWDH445; Lane 3, larger rearranged plasmid; Lane 4, re-isolated pWDH445; Lane 5, smaller rearranged plasmid. Bands representing fragments sized ˜1.8 kb and ˜4.1 kb are present in all four lanes (lanes 2-5) but the band representing a fragment of 1.3 kb (present for pWDH445 and re-isolated pWDH445) is not exhibited in the digestion of either rearranged plasmid (lanes 3 and 5).

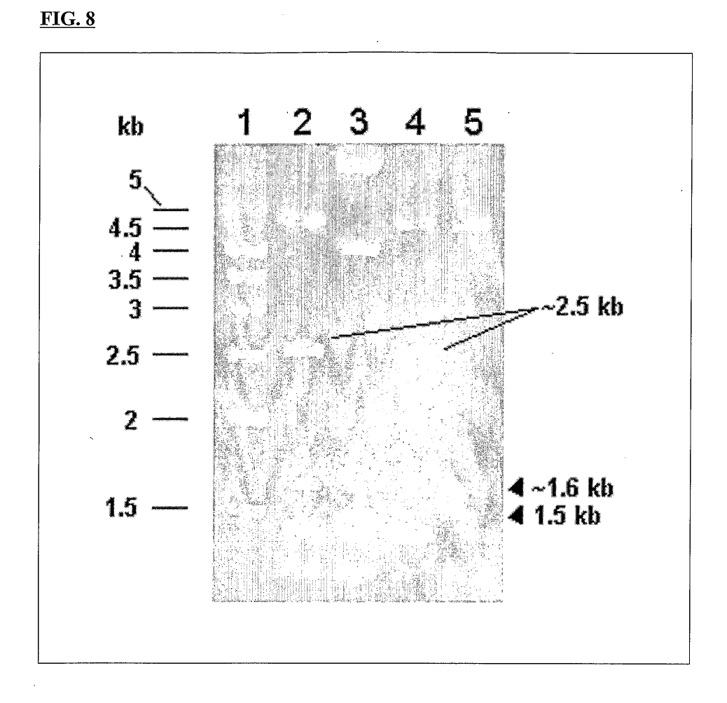

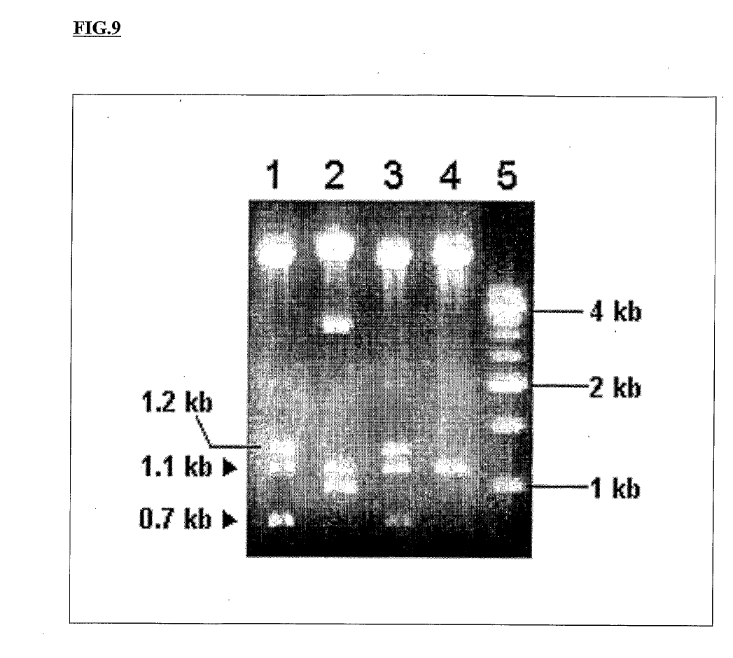

FIG. 8 presents an ethidium bromide stained agarose gel of the four plasmids (pWDH445, re-isolated pWDH445, and two rearranged plasmids) restriction digested with Pst I. Lanes were loaded as follows: Lane 1, 500 bp ladder molecular weight marker DNA; Lane 2, pWDH445; Lane 3, larger rearranged plasmid; Lane 4, re-isolated pWDH445; Lane 5, smaller rearranged plasmid. Lanes 2 and 4 produced identical banding patterns whilst those in lanes 3 and 5 (rearranged plasmids) are significantly different. Only lanes 2 and 4 exhibit a band representing a fragment of ˜2.5 kb. Lanes 2-5 all exhibit bands representing fragments of ˜1.6, ˜1.4, and ˜1.3 kb, but lanes 3 and 5 also showed an extra band representing a fragment of 1.5 kb.

FIG. 9 shows an ethidium bromide stained agarose gel of Pvu I-digested pWDH445, re-isolated pWDH445, and rearranged plasmids. Lanes loaded as follows: Lane 1, pWDH445; Lane 2, larger rearranged plasmid; Lane 3, re-isolated pWDH445; Lane 4, smaller rearranged plasmid; Lane 5, 500 bp ladder molecular weight marker DNA. Lanes 1 and 3 exhibited the expected banding pattern, whereas neither rearranged (lanes 2 and 4) plasmid exhibits bands representing fragments of 1.2 and 0.7 kb. The larger rearranged plasmid (lane 2) also shows two novel bands representing fragments of 3.5 kb and 1 kb.

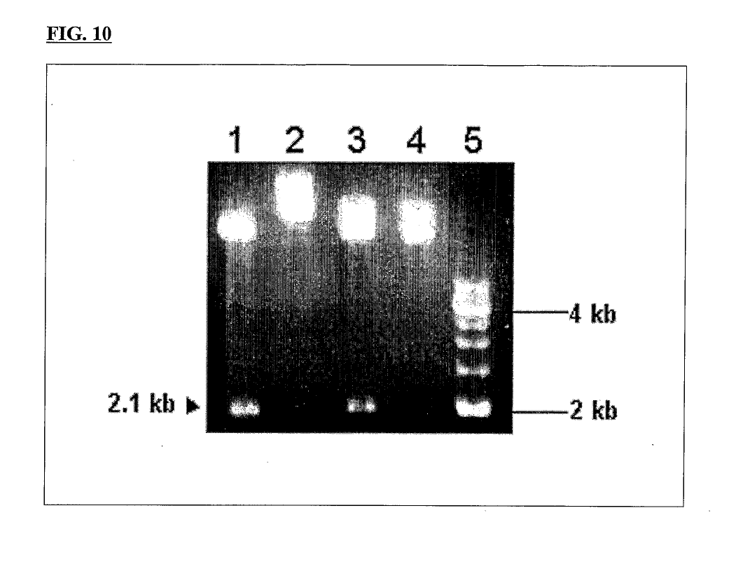

FIG. 10 shows an ethidium bromide stained agarose gel of the four plasmids (pWDH445, re-isolated pWDH445, and two rearranged plasmids) digested with Sac I. Lanes were loaded with the following digested plasmids: Lane 1, pWDH445; Lane 2, larger rearranged plasmid; Lane 3, re-isolated pWDH445; Lane 4, smaller rearranged plasmid; Lane 5, 500 bp ladder molecular weight marker DNA. Sac I liberates a 2.1 kb kanMX internal fragment from pWDH445 and both lanes 1 and 3 exhibit a band representing a 2.1 kb fragment. Neither rearranged plasmid (lanes 2 and 4) reveals such a band after Sac I restriction.

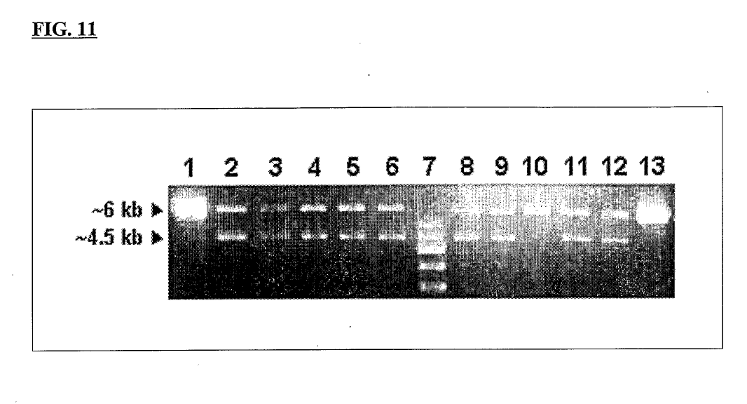

FIG. 11 presents an ethidium bromide stained agarose gel of Xba I-digested pWDH445 and pWDH445 with kanMX enzymically removed. Lanes were loaded as follows: Lanes 1 and 13, pWDH445; Lanes 2-6, 8, 9, 11, and 12, plasmid isolated from ampicillin-resistant, kanamycin-sensitive E. coli transformants; Lane 10, plasmid isolated from an ampicillin-resistant, kanamycin-resistant E. coli transformant; Lane 7, 500 bp ladder molecular weight marker DNA. Lanes 1 and 13 exhibit a doublet band representing two fragments of 6 kb, which is also seen for the plasmid from the kanamycin-resistant colony (lane 10). All other lanes containing digested plasmid show a single band representing a fragment of 6 kb and a second band representing a fragment of −4.5 kb.

FIG. 12 represents the brightness values of FF18984 cells transformed with modified pWDH445 (enzymically removed kanMX) (▪) compared with those from cells transformed with pWDH445 (▪). U, untransformed FF18984 cells not exposed to MMS; U MMS, untransformed cells exposed to 0.005% MMS (15 hours); T, FF18984 cells transformed with pWDH445 or modified pWDH445 but not exposed to MMS; T MMS, cells transformed with pWDH445 or modified pWDH445 exposed to 0.005% MMS; Brightness, fluorescence intensity (F int) corrected for culture density using the intensity of scattered light at 600 nm (Neph600). Each bar represents the average brightness value of ten independent cultures. Cells bearing the modified pWDH445 exhibit enhanced brightness comparable to that of cells re-transformed with isolated rearranged plasmid.

FIG. 13 shows a schematic of the flow-through fluorescence detector with a blown-up schematic of the detector orientations within the light-tight box, and a further blown-up cartoon of the excitation beam and emissions, relative to the flow cell. The top section shows the overall layout of the instrument, beginning with cultures incubating in a water-bath shaker. Alternatively, samples can be injected via the sample injection valve. Culture/sample is pumped peristaltically into the light-tight instrument enclosure (grey box), wherein excitation occurs and emission is detected. The source of the excitation beam is an argon-ion laser (▪). Within the instrument enclosure (see panel blown-up from the grey box) is a duplicated detector set-up. A beam-splitting cube send excitation light (488 nm) to both sets of detectors, each of which consists of an optical flow cell, photomultiplier tube (PMT), silicon photodiode (SPD), and optical filters (see key panel for colour-coding). Fluorescence is detected by the PMT whilst scattered light (as a measure of cell or particulate density) is detected by the SPD, both of which are situated perpendicular to the direction of the excitation beam. Orientation of the excitation beam and the emission detection is simplified in the bottom panel.

FIG. 14 depicts the detection of fluorescence polarisation with the flow-through fluorescence detector. Sample circulating through the flow cell is excited by plane-polarised laser light (vertical arrows represent the plane of polarisation) and scattered light is detected by an SPD placed at the other side of the flow cell in line with the beam of excitation. Emitted fluorescence is less polarised than the excitation light and this is represented by vertical, horizontal, and diagonal arrows around the emission, before reaching the polaroid filters. The two polaroid filters either side of the flow cell are positioned in opposite orientations, such that one filter allows transmission of the vertical component of the fluorescence (Parallel orientation with respect to the plane polarised excitation light), whilst the other permits transmission of the horizontal component (Perpendicular orientation with respect to the plane of polarisation of the absorbed light). Fluorescence transmitted by the polaroid filter is detected by two PMTs (PMT1 and PMT2).

FIG. 15 shows the restriction map of pGen001 a preferred recombinant vector according to the present invention.

FIG. 16 shows full sequence of pGen001 in FASTA format

FIG. 17 shows full sequence of pGen001 in GeneBank format

FIG. 18 represents data from Example 2 illustrating that Methyl methanesulfonate is strongly cytotoxic and genotoxic.

FIG. 19 represents data from Example 2 illustrating that Benzaldehyde is cytotoxic and genotoxic.

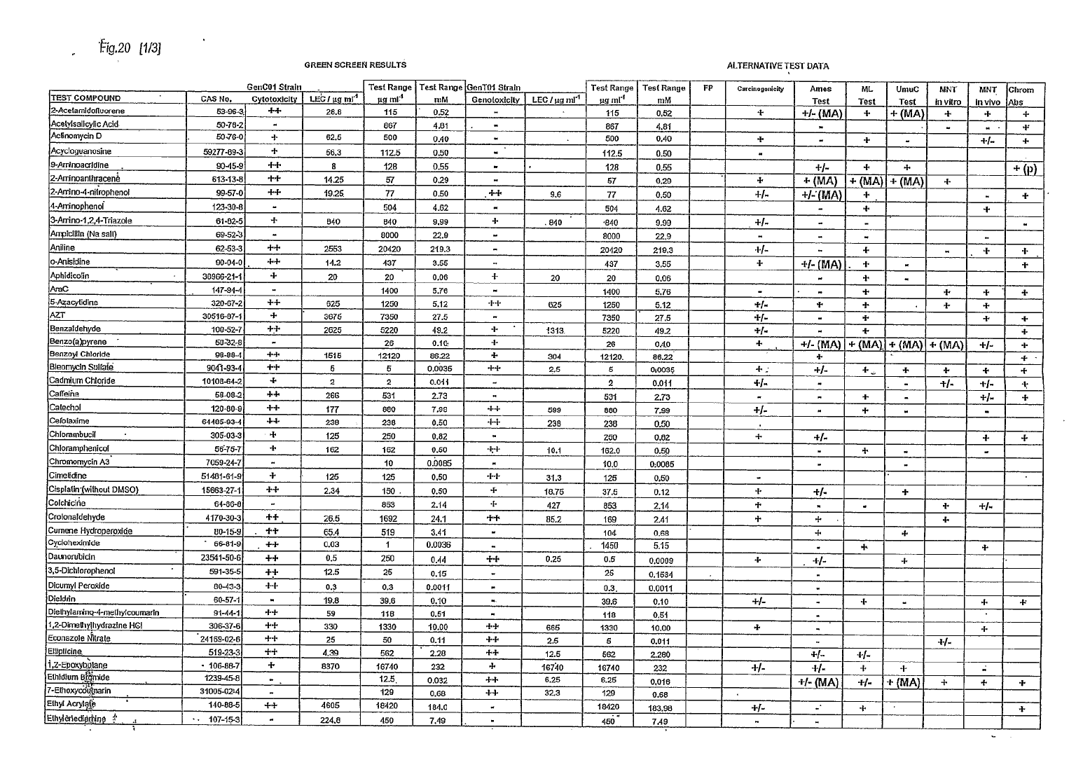

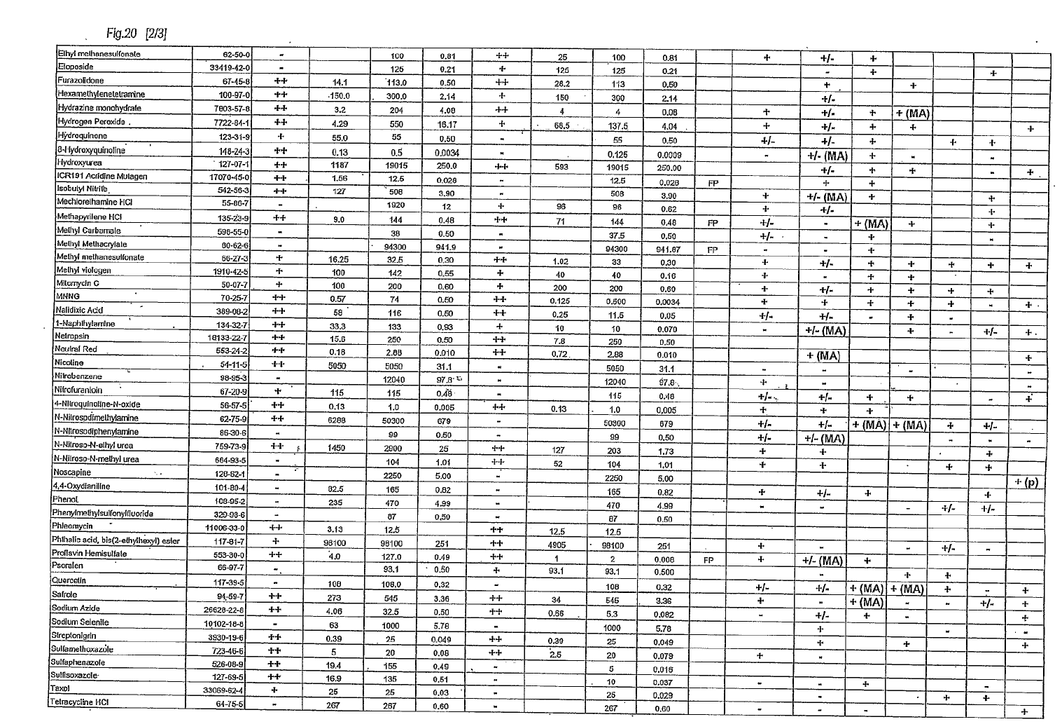

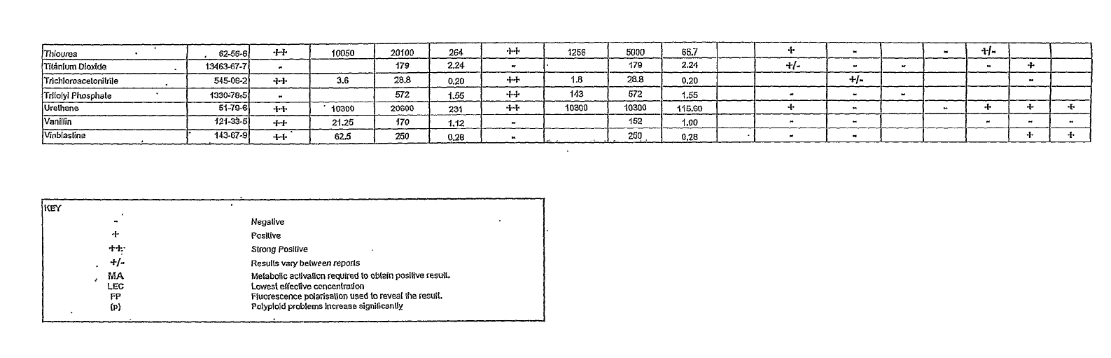

FIG. 20 provides Genotoxicity and cytotoxicity data from the Assay described in Example 2 including test concentration and limits of detection, with comparative data from other genotoxicity tests according to Example 2.

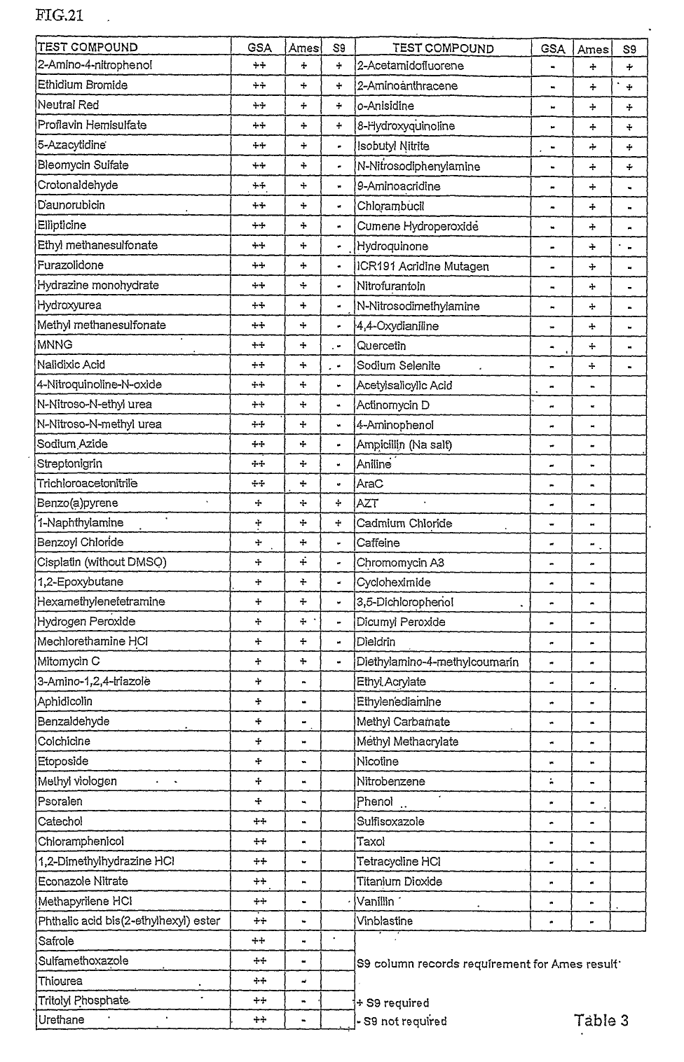

FIG. 21 provides extracted genotoxicity data comparing GreenScreen Assay and reported Ames Test results highlighting compounds requiring S9 metabolic activation for a positive Ames result according to Example 2.

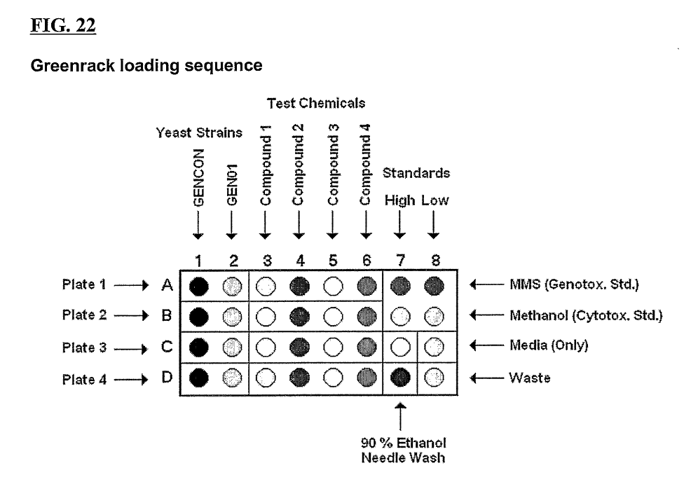

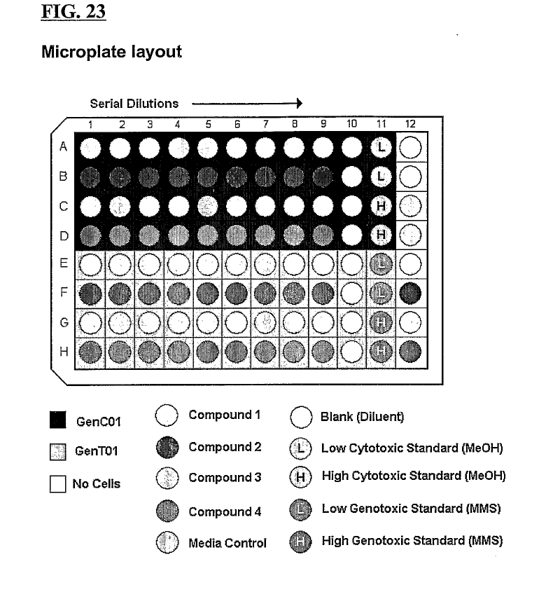

FIG. 22 illustrates the Greenrack loading sequence according to Example 3.

FIG. 23 illustrates a microplate layout according to Example 3.

FIG. 24 shows the restriction map of pGenRNR2 a preferred recombinant vector according to the present invention.

FIG. 25 shows the restriction map of pGenRNR3 a preferred recombinant vector according to the present invention.

FIG. 26 shows the full sequence of pGenRNR2 in GeneBank format.

FIG. 27 shows the full sequence of pGenRNR3 in GeneBank format.

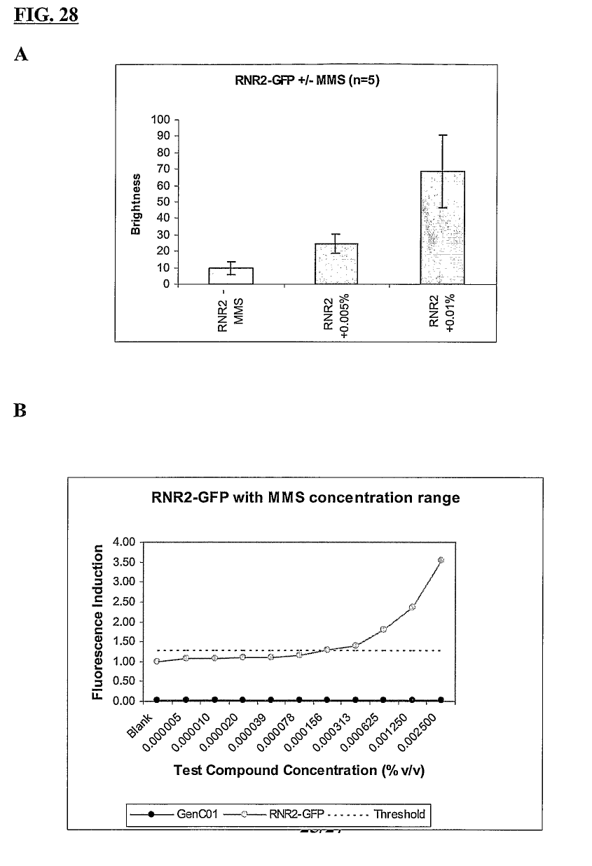

FIG. 28 shows results for (A) a cuvette assay and; (B) a microplate assay for a test strain transfected with pGenRNR2 and using MMS as a test compound in Example 2.

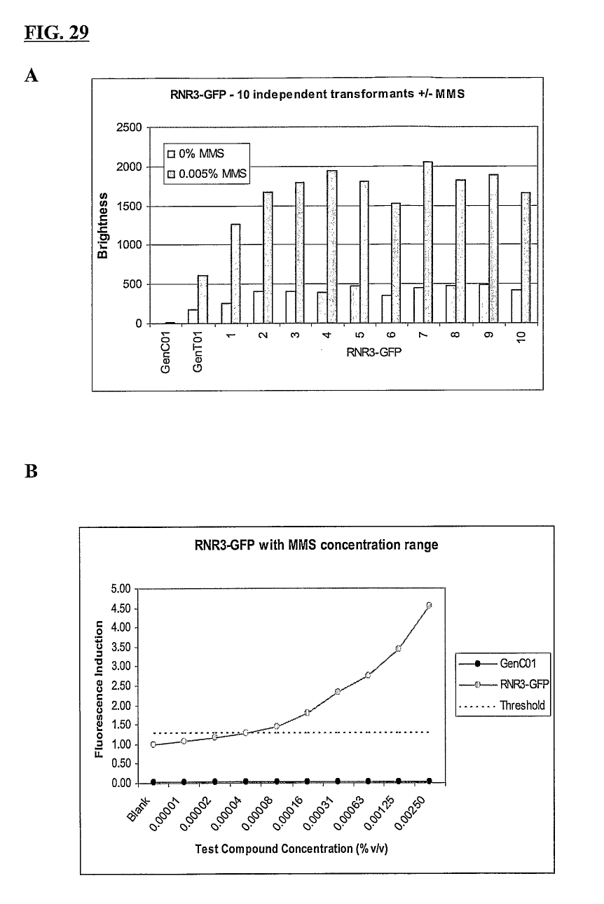

FIG. 29 shows results for (A) a cuvette assay and; (B) a microplate assay for a test strain transfected with pGenRNR3 and using MMS as a test compound in Example 2.

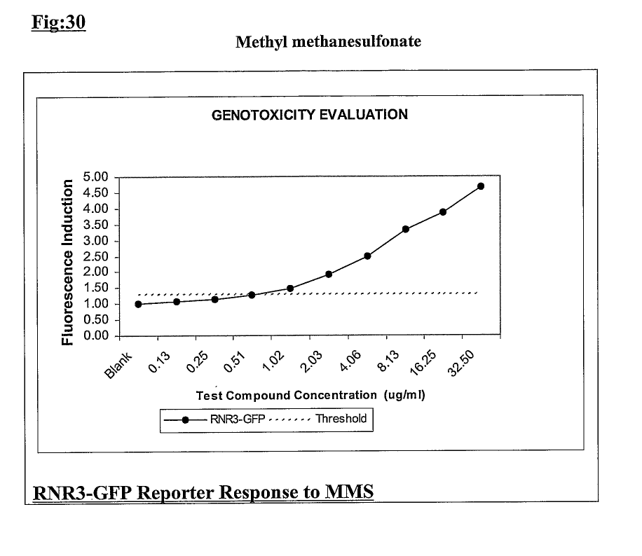

FIG. 30 shows results for a microplate assay for a test strain transfected with pGenRNR3 and using MMS as a test compound in Example 4.

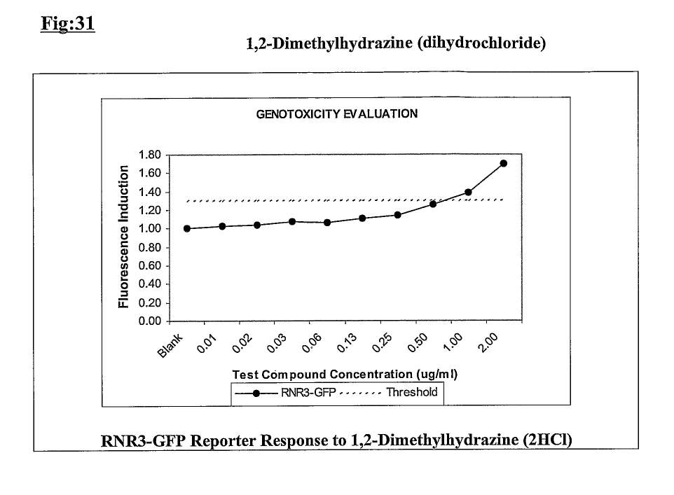

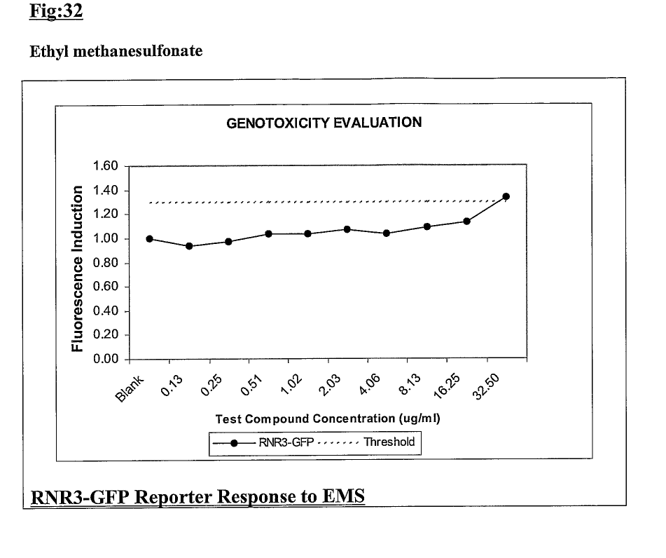

FIG. 31 shows results for a microplate assay for a test strain transfected with pGenRNR3 and using 1,2-Dimethylhydrazine as a test compound in Example 4.

FIG. 32 shows results for a microplate assay for a test strain transfected with pGenRNR3 and using Ethyl methanesulfonate as a test compound in Example 4.

FIG. 33 shows the full sequence of RNR3 sequence including 1 kb upstream of start codon in GeneBank format.

FIG. 34 shows the removal of bacterial origin of replication and Amp resistance from pGen001 to generate pGenEM001, a preferred vector according to the invention.

FIG. 35 shows a fragment of HO sequence.

FIG. 36 shows the restriction map of pGenIn012, a preferred recombinant vector according to the present invention.

FIG. 37 shows rDNA sequence used in multiple copy rDNA integrating plasmids according to the invention.

FIG. 38 shows the restriction map of pGenIn022A, a preferred recombinant vector according to the present invention.

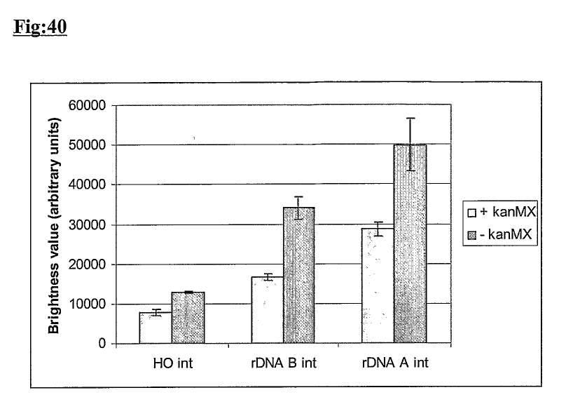

FIG. 39 shows the difference between the brightness values obtained from empty pRS316 vector compared with pRS316 containing the RAD54-GFP reporter cassette plus the kanMX module, and compared with pRS316 containing the RAD54-GFP cassette but not kanMX.

FIG. 40 show the effect of the presence of kanMX on the brightness of RAD54-GFP (all without MMS) when integrated into the chromosome at HO (HO int) and the rDNA array (rDNA B int and rDNA A int-A and B refer to orientation).

FIG. 41 shows the full sequence of pGenIn012 in GeneBank format.

FIG. 42 shows the full sequence of pGenIn022A in GeneBank format.

### EXAMPLE 1

The discovery of brighter cells carrying a mutant pWDH445 will be described. This brighter strain was compared with a typical pWDH445-bearing strain followed by phenotypic analysis of the new strain and molecular biological examination of the plasmid borne by these cells. Finally, the creation of brighter strains by basic genetic engineering is disclosed.

### Materials & Methods

### Strains and Plasmids

### Strains

Saccharomzyces cerevisiae and Escherichia coli strains used in the Example are listed in Table 1, along with their respective genotypes.

TABLE 1

| Strain | Organism | Genotype | Source |
|---|---|---|---|
| FF18984 | S. cerevisiae | MATa leu2-3, | F. Fabre |
| | | 112 lys2-1 his7-1 ura3-52 | |
| DH5α | E. coli | SupE44 ΔlacU169 (φ80lacZΔ15) | Laboratory |
| | | hsdR17 recA1 endA1 gyrA96 | collection |
| | | thi-1 relA1 | |

### Plasmids

pWDH445—is illustrated in FIG. 5 and corresponds to yEGFP-444 as disclosed in WO 97/44149 (e.g. see FIG. 12)

### Media

### Sterilisation

All media were prepared by dissolution of the components in distilled water before sterilisation by autoclaving for 20 minutes at 15 lbin² unless otherwise stated. Alternatively, sterilisation was performed by filtration through 0.22 μm diameter pore Millipore filters where autoclaving was inappropriate.

### Yeast Media

Bacto-agar was added to a final concentration (w/v) of 2% to each of the growth media. Yeast extract, peptone, and agar were all obtained from Difco (Becton Dickinson, Sparks, Md. 21152, USA). The following media were used (Sherman et al. “Methods in yeast genetics” Cold Spring Harbor Laboratory Press).

### YPD (Yeast Extract, Peptone, and Dextrose)

TABLE 2

| Component | % w/v | Final concentration gL⁻¹ |
|---|---|---|
| Bacto-yeast extract | 1 | 10 |
| Bacto-peptone | 2 | 20 |
| Dextrose (glucose) | 2 | 20 |
| *Bacto-agar | 2 | 20) |

*Bacto-agar added in preparation of solid medium.

### YPG (Synthetic Dextrose)

TABLE 3
YPG

| | | Final concentration |
|---|---|---|
| Component | % w/v or v/v | gL⁻¹ or mlL⁻¹ |
| Bacto-yeast extract | 1 | 10 |
| Bacto-peptone | 2 | 20 |
| Glycerol | 3 (v/v) | 30 ml |
| *Bacto-agar | 2 | 20 |

*Bacto-agar added in preparation of solid medium.

### SD

TABLE 4
SD medium

| Component | % w/v | Final concentration gL⁻¹ |
|---|---|---|
| **Bacto-yeast nitrogen | 0.17 | 1.7 |
| base | | |
| (NH₄)₂SO₄ | 0.5 | 5 |
| Dextrose (glucose) | 2 | 20 |
| *Bacto-agar | 2 | 20 |

*Bacto-agar is added in the preparation of solid medium.
**Bacto-yeast nitrogen base was purchased without amino acids or ammonium sulphate. This allows control of the nitrogen source and facilitates the preparation of selective media. A 20x YNB stock comprising 34 gL⁻¹ Bacto-yeast nitrogen base and 100 gL⁻¹ (NH₄)₂SO₄ was stored in the dark at 4° C.

TABLE 5
SD Supplements

| | Working | | |
|---|---|---|---|
| | concentration | Stock solution | Volume of stock |
| Component | mgL⁻¹ | g/100 ml | mlL⁻¹ |
| Adenine | 20 | 0.2 | 10 |
| sulphate | | | |
| Uracil | 20 | 0.2 | 10 |
| L-histidine | 20 | 1.0 | 2 |
| L-leucine | 30 | 1.0 | 3 |
| L-lysine | 30 | 1.0 | 3 |
| L-tryptophan | 20 | 1.0 | 2 |

### F1 Medium (Fermentation One)

TABLE 6
F1 medium - salts stock (6.7×)

| Component | [×6.7] gL⁻¹ | Final conc. gL⁻¹ |
|---|---|---|
| (NH₄)₂SO₄ | 20.88 | 3.13 |
| KH₂PO₄ | 13.33 | 2.00 |
| MgSO₄•7H₂O | 3.66 | 0.55 |
| CaCl₂•2H₂O | 0.60 | 0.09 |
| NaCl | 0.67 | 0.10 |

TABLE 7
F1 medium - trace elements stock (10000×)

| Component | [×10000] gL⁻¹ | Final conc. mgL⁻¹ |
|---|---|---|
| ZnSO₄•7H₂O | 0.7 | 0.07 |
| FeCl₃•6H₂O | 0.5 | 0.05 |
| CuSO₄•5H₂O | 0.1 | 0.01 |
| H₃BO₃ | 0.1 | 0.01 |
| KI | 0.1 | 0.01 |

TABLE 8
F1 medium - vitamins stock (600×)

| Component | [×600] gL⁻¹ | Final conc. mgL⁻¹ |
|---|---|---|
| Inositol | 37.2 | 62 |
| Thiamine/HCl | 8.4 | 14 |
| Pyridoxine | 2.4 | 4 |
| Ca-pantothenate | 2.4 | 4 |
| Biotin | 0.18 | 0.3 |

F1 medium is a defined minimal medium used in fermentation [Brown et al, 1981, adapted by Walmsley et al., 1983] that has a particularly low background autofluorescence. Concentrated stocks of salts, trace elements and iron (III) chloride were autoclaved, stored separately, and diluted into sterile water upon requirement. A concentrated vitamin stock was prepared in sterile water, filter sterilised by syringe and sterile 0.20 μm pore filter (Sartorius, Gottingen, Germany), and stored in aliquots at −20° C. Vitamins were then added subsequent to autoclaving of F1, to avoid their denaturation. All F1 was stored at 4° C. in the absence of glucose.

A subsequent variant form of F1 was employed with 50% less inositol than quoted in the vitamins stock Table 8 in order to lessen the effects of flocculation in troublesome yeast strains. This reduced inositol F1 was further modified by producing it in phosphate buffer at pH6 (as described in Varley, 1967. Practical Clinical Biochemistry, 4th Edition, William Heinemann Medical Books, pp. 759), instead of in water. The buffered medium was created by mixing 0.067 M solutions of KH₂PO₄ and Na₂HPO₄, or concentrated stocks thereof (see Table 9), before addition of salts, trace elements, iron chloride, and nutritional supplements. The buffer mix was autoclaved and allowed to cool down to room temperature before addition of vitamins.

TABLE 9
Phosphate buffer for F1 medium

| | Volume of 1/15 M PO₄²⁻/100 ml |
|---|---|
| Component | Ml |
| Na₂HPO₄ | 12.2 |
| KH₂PO₄ | 87.8 |

### Bacterial Media

### LB Medium

TABLE 10
LB Medium

| Component | Concentration gL⁻¹ |
|---|---|
| Bacto-tryptone | 10 |
| Bacto-yeast extract | 5 |
| NaCl | 10 |

Luria-Bertani medium (LB) for broth cultures was prepared as described [Sambrook et al., second edition, 1989] and LB broth plus 2% Bacto-agar was used for solid cultures.

### Antibiotics

Ampicillin

Ampicillin was prepared in aqueous solution at a stock concentration of 10 mgml⁻¹ and used at working concentrations of up to 100 μgml⁻¹. Ampicillin stock solutions were stored in aliquots of 1 ml at −20° C.

### Geneticin (G418)

G418 disulphate salt was prepared in aqueous solution as a stock solution at the concentration of 20 mgml⁻¹ and used at a working concentration of 200 μg/ml. The G418 stock solution was stored at 4° C.

### Kanamycin

Kanamycin was also prepared in aqueous solution, at a stock concentration of 50 mgml⁻¹ and used at a working concentration of 50 μgml⁻¹. Kanamycin stock solutions were stored at −20° C.

Standard solutions and buffers used herein were taken from Sambrook et al (second edition, 1989).

Standard yeast and E. coli techniques, for example, biomass production, transformation, isolation of plasmid DNA, and restriction enzyme digests, were used in accordance with published methods.

### Fluorescence Detection and Quantitation

### Fluorescence Assays with Cell Extracts

The assessment of GFP fluorescence in cell extracts was performed essentially as described in Walmsley et al., 1997 (Yeast 13 p 1535-1545). A stationary phase culture of cells grown in SD medium was used as the inoculum source. 15 μl aliquots of cells were inoculated into 1.5 ml of SD in 15 ml test tubes. Half of the tubes were then supplemented with 0.01% MMS (the remaining tubes left as controls). The tubes were incubated at 25° C. (accurate folding of the S65T derivative of GFP is temperature sensitive) for 16 hours in an orbital shaker incubator at 120 rpm, or in a water bath shaker. After 16 hours the cultures were adjusted to 0.02% NaN₃ to inhibit respiration and incubated on ice for 90 minutes, maintaining agitation. The cells were then transferred to 1.5 ml microfuge tubes, harvested by centrifugation (10 s), washed twice in sterile distilled water, then washed in 1 ml of “extraction” buffer (20 mM Tris-Cl pH 7.5, 0.1 M NaCl, and 1 mM EDTA). After harvesting the cells by centrifugation, the supernatant was aspirated off and the cell pellet resuspended in 250 μl “crushing” buffer (20 mM Tris-HCL, pH 7.5, 0.1 M NaCl, and 1 mM PMSF). 250 μl of 400-600 nm diameter glass beads were added to the tubes of cells that were then placed in a BIO101 Fastprep FP120 (Savant, purchased from Anachem Ltd, Luton, UK) on speed 4 for 30 seconds to mechanically disrupt the cells. The tubes were incubated on ice for 1 minute and then returned to the Fastprep for another 30 seconds. This process was repeated such that each tube had undergone 3 periods in the Fastprep separated by 1 minute ice incubations. Following centrifugation for 30 s, the supernatant was transferred to a clean tube. The pellet of beads was washed in a further 250 μl of crushing buffer and this was added to the supernatant from the previous extraction. The extract was adjusted to 0.1 M Tris, pH 11 by the addition of 1 M Tris base, 200 μl of the pooled extract plus 2.8 ml of water was then transferred to an acrylic cuvette (Sarstedt Ltd, Numbrecht, Germany).

### Fluorescence Assays with Intact Yeast Cells

Starter cultures were initiated by inoculating 1.5 ml of F1 medium (plus appropriate nutritional supplements and 2% glucose) in sterile 15 ml polythene centrifuge tubes with small portions of colonies, picked by inoculating loop from stock plates. These starter cultures were grown for up to 24 hours at 30° C. with shaking (at 120 rpm) and were used as the source of inoculum for the assay cultures. Cells were inoculated into 1.5 ml F1 cultures in 15 ml polythene centrifuge tubes to give an initial OD_{600 nm} of ˜0.1 (typically a 10-15 μl inoculum per 1.5 ml culture). The lids of the tubes were left one-quarter unscrewed, but held in place by masking tape, to ensure maximum oxygenation for GFP maturation. Half of the tubes were treated with 0.005% methyl methanesulphonate (MMS, methanesulphonic acid methyl ester) and half left as unchallenged controls. MMS was purchased as a liquid and this stock solution was taken to be 100%. This was diluted to a 0.5% stock in small aliquots as required and then diluted 1:100 for the assay cultures (15 μl of 0.5% stock per 1.5 ml culture). Assay cultures were incubated at 25° C. (30° C. when the S65T-GFP was replaced by yEGFP, which is more heat stable) in shaking water bath incubators for 14-16 hours. Each of the 1.5 ml cultures was transferred to a 4-window acrylic cuvette (Sarstedt Ltd, Numbrecht, Germany) and diluted with 1 ml of sterile water before measurement. For the measurement of intact yeast cell fluorescence of cells grown in YPD or SD, the cells were washed twice in sterile water to remove traces of autofluorescent medium and resuspended in 1.5 ml of sterile water. Washed cells were then transferred directly to 4-window acrylic cuvettes containing 1 ml of sterile distilled water. 4-window cuvettes were necessary since measurement of fluorescence emission is performed perpendicular to the path of excitation light.

### Fluorescence Measurements and “Brightness” Calculation

Fluorescence measurements were performed with a Perkin-Elmer LS50B Fluorescence Spectrometer (Perkin-Elmer Ltd., Beaconsfield, UK). The excitation and emission wavelengths were set to 488 nm and 511 nm respectively, with a slit width of 10 μm, for the S65T GFP derivative-expressing reporter. For the yEGFP expressing reporter, the excitation and emission wavelengths were set to 490 nm and 518 nm respectively (due to the altered fluorescence characteristics of yEGFP), with 5 nm slit widths. To account for differences in cell numbers between YPD or SD assay cultures, the OD_{600 nm} was recorded for each cuvette. Similarly, in order to measure protein extraction efficiency, light absorption at 280 nm (OD_{280 nm}) was measured for crude cell extracts. Both of these measurements used a Cecil Instruments CE505 double beam ultraviolet spectrophotometer. Acrylic cuvettes were essential for the latter measurements since they permit greater transmission of ultraviolet (UV) light. Cell density estimations for F1 medium cultures were made by measuring light scattering (nephelometry) using the LS50B luminescence spectrometer, with both excitation and emission wavelengths at 600 μm and slit widths of 2.5 nm (Neph600). The fluorescence values obtained from the fluorometer were then divided by the absorption/scatter readings to give the “brightness value”, an arbitrary unit which is independent of sample concentration, though varies with different fluorometers. The y-axis on uncorrected fluorescence scans gives raw data in the form of machine defined fluorescence intensity (“INT”) units.

### Induction Ratio

The induction ratio is used to calculate a “signal-to-noise ratio” with respect to the GFP signal. All detected signals are not pure GFP signals, but incorporate contaminating background fluorescence signals (autofluorescence). Autofluorescence significantly varies with the changing growth phase of a culture and is dependent on the strain background. Hence, in order to be able to compare the signal-to-noise ratios from cultures of the same strain in different growth phases or from cultures of different strains, it is necessary to remove the variable autofluorescence component from the brightness values.

Firstly, several terms (used throughout this thesis) must be defined:

“U” is the brightness value from untransformed cells i.e. cells not bearing a reporter plasmid (or other plasmid where specified), that have not been exposed to a genotoxin such as MMS, and as such this value represents the uninduced autofluorescence.

“U MMS” is the brightness value from untransformed cells that have been exposed to a genotoxin (in this case, MMS). This value represents the genotoxin-induced level of autofluorescence, often larger than that of uninduced autofluorescence.

“T” is the brightness value from cells transformed with one of the RAD54-GFP reporter plasmids (or other plasmid where specified) that have not been exposed to a genotoxin. This value represents the constitutive level of GFP fluorescence due to continual low-level expression from the RAD54 promoter, and also incorporates the uninduced autofluorescence.

“T MMS” is the brightness value obtained from cells transformed with a plasmid that have been exposed to a genotoxin (again, MMS in this case). T MMS represents a combination of the GFP signal due to damage-induced up-regulation of expression from the RAD54 promoter, the constitutive GFP signal, and the damage-induced autofluorescence signal.

For a true signal-to-noise ratio based solely on the signal from GFP, the two parameters of interest are the GFP signal due to the damage-induced response of RAD54 and the constitutive GFP response in the absence of damage. The constitutive signal is obtained by subtracting the uninduced autofluorescence from T (i.e. T-U) and the induced GFP signal is calculated by subtracting the induced autofluorescence from T MMS (i.e. T MMS-U MMS). (T-U) gives the constitutive signal or C value, whereas (T MMS-U MMS) provides the induced value or I value. The induction ratio is a simple ratio of I to C and can be expressed thus:

$$Induction Ratio = \frac{( T MMS - U MMS )}{( T - U )} \frac{( GFP signal )}{( GFP noise )}$$

### Flow-Through Fluorescence Detector

The development of a continuous-flow detector for the measurement of GFP signal from a yeast culture has been described previously [Knight et al., 1999, Measurement Science and Technology, 10: 211, 217] and adapted for the fluorescence polarisation technique [Knight et al., 2000a, The Analyst, 125: 499-506]. FIG. 13 shows a schematic diagram of the basic layout of the instrumentation developed with a fluorescence flow cell, through which the yeast culture or GFP extract is circulated by use of a Gilson Minipuls 3 peristaltic pump (purchased from Anachem Ltd, Luton, UK). The 488 nm excitation was provided by an air-cooled 40 mW argon ion laser (LG Laser Graphics GmbH, Dieberg, Germany), reduced to 5 mW by filtering. A photomultiplier tube (PMT) was used as the fluorescence detector and a silicon photodiode (SPD) as the scattered light detector for nephelometric measurement of the cell density. The flow cell, PMT, SPD, and associated electronics were housed in a light-tight box (in duplicate in FIG. 13). The PMT is positioned to one side of the flow cell such that fluorescence is detected at 90° to the path of the excitation light. The SPD is situated at the other side of the flow cell from the light source in the path of the excitation light, or to one side of the flow cell in the same way as the PMT, since scattered light can detected in either orientation. Data acquisition and manipulation was performed in real time on a personal computer via a 12-bit analogue to digital converter (ADC) and associated software (Pico Technology Ltd., Cambridge, UK).

### Fluorescence Polarisation

FIG. 14 shows the detection of fluorescence polarisation using the flow-through fluorescence detector. Polarised laser light (polarisation represented by vertical arrows) is used to excite the sample circulating through the flow cell. In this cartoon, fluorescence is detected at 90° on either side of the flow cell with a single polaroid filter between the flow cell and each PMT. Fluorescence from the sample in the flow cell is less polarised than the excitation light (represented by vertical, horizontal, and diagonal arrows). The polaroid filters to either side of the flow cell are arranged in opposite orientations, such that one filter allows the vertical component of the fluorescence to pass through (parallel orientation, with respect to the plane of polarisation of the incident light), while the other permits transmission of only the horizontal component of the fluorescence (perpendicular orientation, with respect to the plane of polarisation of the incident light).

The degree of fluorescence polarisation (P) measured in this work was defined as:

P=(I_{∥}−I_{⊥})/(I_{∥}+I_{⊥})

where I_{∥} is the fluorescence intensity measured polarised parallel to the absorbed plane-polarised radiation, and I_{⊥} is that perpendicular to the absorbed radiation. P is a dimensionless parameter and is not dependent on the intensity of the emitted light or on the concentration of the fluorophore. For the discrimination of GFP signal from that of autofluorescence, calculation of the (I_{∥}−I_{⊥}) term is sufficient, since this term tends to zero for molecules emitting relatively unpolarised fluorescence (for example, small molecules like fluorescein), whilst being relatively large for molecules emitting fluorescence that exhibits significant polarisation.

### Results

### Brighter FF18984 Transformants Bearing pWDH445

### Spontaneous Generation of Bright pWDH445-Bearing FF18984 Cells

After the production of new pWDH445-containing strains, transformants screened by marker selection were assayed for a change in brightness in response to MMS, to ensure the presence of functional pWDH445.

Sporadically, pWDH445 transformants (transformed using standard experimental procedures) were identified which were significantly brighter in response to MMS than typical transformants. FIG. 1 shows the average normal brightness for 3 replicates of FF18984 cells carrying pWDH445 (darker bars) and the average brightness for 3 replicates of brighter FF18984 cells carrying pWDH445 (lighter bars). In this example, ‘T’ (transformed cells with no MMS) was 12 fold brighter in the new variant FF18984 cells than the typical signal level, and ‘T MMS’ was 9 fold brighter. The enhanced brightness in the variant cells actually led to a decrease in the induction ratio from 7.72 to 3.27. However, the ease of detection of the variant cells with enhanced signal output made them attractive for the development of ever-brighter reporters.

### There is a Link Between Enhanced Signal Output and Geneticin Resistance

The bright variants discussed above were first subjected to phenotypic analysis to confirm that they were indeed FF18984 transformants. Strains were re-plated onto media excluding uracil to check for the presence of the pWDH445. This was the favoured method of transformant selection to minimise the use of antibiotics (in this case G418). Both normal and variant transformed cells grew on SD medium lacking uracil. Cells were replica-plated onto SD medium lacking one of leucine, lysine, or histidine, and once more both normal and variant strains responded accordingly: i.e. they were unable to grow. This suggested that both normal and variant cells had the correct genetic background for FF18984.

The cells bearing pWDH445 were then tested for resistance to G418, which is conferred by the kanMX module carried on pWDH445. Cells of typical brightness were G418 resistant and hence able to grow on G418 (200 μgml⁻¹) containing YPD plates. However, bright variant cells were unable to grow on such medium, despite the previous confirmation of the presence of pWDH445 by growth on uracil-deficient medium. This suggested that mutation or rearrangement of the reporter plasmid had occurred in the variant cells.

### The RAD54-GFP Cassette has No Gross Rearrangements

In order to test the hypothesis that the plasmid had undergone a rearrangement or mutation, it was necessary to isolate pWDH445 from both normal cells and variant cells. Plasmid was prepared from yeast using standards techniques and the DNA pellet was dissolved in 40 μl of sterile water. 5 μl of each plasmid preparation was used to transform DH5α E. coli cells using standard techniques. E. coli transformed with the plasmids prepared from yeast were selected by growth on LB agar containing 80 μgml⁻¹ ampicillin. Transformant cultures and plasmid preparations were made as described in the methods and DNA pellets were resuspended in 50 μl of TE and RNAase (20 μgml⁻¹).

A simple test for the integrity of the RAD54-GFP cassette in pWDH445 was to utilise the modular nature of the plasmid to liberate the cassette by restriction endonuclease digestion. This module is released by double digestion of pWDH445 with BamH I and Asc I restriction enzymes to give a 3177 bp fragment from the normal plasmid. pWDH445, plasmid prepared from 2 independent bright transformants, and plasmid isolated from a normal transformant were digested with BamH I and Asc I, and the resulting DNA fragments separated by agarose gel electrophoresis. FIG. 2 shows the bands produced after ethidium bromide staining for the above restriction digests. All 4 pWDH445 reactions produce a band of 3.2 kb suggesting that the HO-RAD54promoter-yEGFP cassette is unchanged from the normal pWDH445 (lanes 1 and 3) to those isolated from bright variants (lanes 2 and 4). However, the band representing the remainder of the plasmid is larger in one of the variants (lane 2) and smaller in the other (lane 4), whilst it remains constant between the DNA used in transformation and that isolated from a transformant showing normal brightness levels.

### Yeast Re-Transformed with Rearranged Plasmid are G418 Sensitive

Plasmid DNA isolated from yeast and then prepared from E. coli after amplification was used to re-transform FF18984 yeast with normal and rearranged pWDH445, using the lithium acetate/PEG/SS-DNA protocol. Transformants were selected by their ability to grow on medium deficient in uracil, since this was apparently unaffected by the mutations/rearrangements in the variants. The auxotrophic markers of transformants were checked as before and corresponded to those of the FF18984 background, with the exception of the ability to grow without uracil, conferred by pWDH445. Once more, resistance to G418 was tested by transferal of transformants to YPD plates containing 200 μgml⁻¹ G418. FIG. 3 shows photographs taken of re-transformed FF18984 incubated on YPD plates containing G418 and SD plates lacking uracil. The uracil-deficient plates are represented in the right-hand column and as expected demonstrate growth in all cases. However, the G418 plates (left-hand column) reveal that cells carrying the control pWDH445 and the normal pWDH445 isolated from yeast are G418 resistant as expected, but cells bearing either of the 2 anomalous plasmids are sensitive to 200 μgml⁻¹ G418. This suggests that the mutations or rearrangements are stable in that they were not reverted by the mutational effects sometimes associated with the transformation procedure.

### Re-Transformed G418-Sensitive Yeast Still Give Brighter Signal Outputs

FF18984 cells re-transformed with the altered reporter plasmids were tested for their ability to induce the reporter in response to 0.005% MMS, compared with cells carrying the normal pWDH445. After 15 hours incubation with MMS, fluorescence and scatter measurements were made and brightness values determined. FIG. 4 depicts the brightness signals from both cells bearing normal pWDH445 (darker bars) and cells conveying the altered plasmids (lighter bars). As previously, the lighter bars for the transformed cells are significantly larger than the corresponding darker bars. The modified plasmids gave rise to a 3.3 fold increase in the untreated brightness signal and a 4.6 fold increase in the MMS-induced signal, compared with cells bearing the unchanged pWDH445. Overall these increases had little effect on the induction ratio, producing only a slight decrease from 5.5 to 5.2 in cells bearing modified plasmid. However, the increase in brightness signal with the re-transformants was not as great as for the original variants isolated.

### Restriction Analysis of pWDH445 and its Brighter Derivatives

It was hypothesised that there was a link between the enhancement of brightness signal and the loss of G418 resistance, which suggested a change in or the loss of the kanMX module. Investigation of this required the use of diagnostic restriction digestions.

A restriction map of pWDH445 defined in this study is shown in FIG. 5.

### Xba I Digestion Revealed Size Differences in the Rearranged Plasmids

The BamH I and Asc I double digestion employed previously to confirm the presence of the intact HO-RAD54 promoter-yEGFP module also revealed that one rearranged plasmid was larger than pWDH445 whilst the other was smaller. Xba I digestion was used to confirm the size changes in the rearranged plasmids after fractionation of the DNA fragments by agarose gel electrophoresis. From the map, Xba I digestion to completion was expected to yield 2 bands in a doublet formation at ˜6 kb, for pWDH445 and plasmid isolated from cells exhibiting normal brightness. In FIG. 6, lanes 1 and 3 represent the fragments created by Xba I digestion of pWDH445 and plasmid from cells of normal brightness, separated on a 1% agarose gel and labelled with ethidium bromide. Both lanes show identical bands in a doublet formation at an approximate size of 6 kb (lane 5 contains 500 bp ladder molecular weight marker DNA, with the largest fragment at 5 kb). Lane 4 represents the Xba I fragments for the rearranged plasmid that appeared to be smaller in the BamH 1-Asc I double digestion. The smaller size is also reflected in the replacement of the larger band from the doublet (˜6 kb) with a band at 4.4 kb, giving a vector size of ˜10.4 kb as opposed to the 12 kb of pWDH445. Lane 2 shows the Xba I fragments for the other rearranged plasmid that appeared to be larger than pWDH445 in the BamH I-Asc I digestion. As with the smaller rearranged plasmid, the larger band from the 6 kb doublet is lost, but replaced not only with a band of ˜4.4 kb, but also 2 further new bands at ˜3.2 and 3.0 kb. This would suggest a vector size of ˜16.6 kb, an increase of ˜4.6 kb compared with pWDH445. This suggests that the brightness increase in cells carrying the rearranged plasmids was not purely a vector size-related phenomenon (commonly there is an inverse relationship between size and copy number). It is also suggested that both rearrangements occur in the same half of the vector since the larger of the ˜6 kb bands is lost in each case. The larger band represents the half of the plasmid bearing Amp^{r}, kanMX3, and yEGFP. This further corroborated the suggestion from the BamH I-Asc I digestion, that the HO-RAD54 promoter-yEGFP module was intact after rearrangement.

### Sca I Digestion Further Limited the Region of Rearrangement

pWDH445 contains four recognition sites for the Sca I restriction enzyme and hence Sca I digestion should result in the production of four fragments. The four fragments should be represented by bands at approximately 4.7, 4.1, 1.9, and 1.3 kb after electrophoretic separation. FIG. 7 shows a picture of the ethidium bromide stained 1% agarose gel used to fractionate the Sca I fragments from pWDH445 (lane 2), the larger rearranged plasmid (lane 3), re-isolated pWDH445 (lane 4), and the smaller rearranged plasmid (lane 5). Lanes 2 and 4 show the same patterns of bands, with a doublet between 4 and 5 kb (representing the 4.7 and 4.1 kb fragments) and single bands at ˜1.8 kb and 1.3 kb. Neither of the rearranged plasmids (lanes 3 and 5) exhibit the 1.3 kb Sca I band, the production of which requires the Sca I recognition site within kanMX3 to be intact. The ˜1.8 and 4.1 kb bands are present in all 4 of the plasmids digested, implying that no alteration had occurred in the region containing HO, URA3, the 2 micron origin, or Amp^{r}. Hence, the portion of the vector in which the rearrangements could have occurred was further limited to the region between the unique Asc I recognition site and the Sca I recognition site in Amp^{r}, including the entire kanMX3 module.

### The Pst I Cleavage Site Within the kanMX3 Module is Lost in the Rearranged Plasmids

Once the region of the plasmid incorporating the rearrangements was established, it was necessary to test restriction enzymes with cleavage sites in this section. The Pst I cleavage site occurs 7 times in pWDH445, though only 6 bands were expected to be detectable on an ethidium bromide stained 1% agarose mini-gel after electrophoretic separation of the Pst I fragments. The seventh fragment was too small to be detected under these conditions. Approximate sizes for the 6 bands were 4.2, 2.5, 2.2 or 1.6, 1.4, 1.3, and 0.9 kb. The fragment expected to be detected at 2.2 or 1.6 kb should result from cleavage at the sites within HO and URA3, but the size is dependent upon the orientation of the URA3 gene.

FIG. 8 shows a picture of the ethidium bromide stained gel in which the fragments from Pst I digested plasmid were separated. pWDH445 and re-isolated pWDH445 were loaded into lanes 2 and 4, respectively, whilst the larger and smaller rearranged plasmids were loaded into lanes 3 and 5, respectively. All four plasmids exhibit a 1.6 kb band suggesting that the URA3 gene is transcribed in the opposite direction to the HO-RAD54 promoter-yEGFP-kanMX3 cassette. The 2 bands involving the Pst I cleavage site in the kanMX3 module are the 2.5 and 0.9 kb fragments. Only lanes 2 and 4, containing digested pWDH445 and digested re-isolated pWDH445, produced bands at 2.5 and 0.9 kb (the 0.9 kb band is not visible in FIG. 8) whilst these were absent for both rearranged plasmids (lanes 3 and 5). Loss of the 2.5 kb and 0.9 kb bands from both of the rearranged plasmids suggests that a rearrangement had occurred within kanMX, causing loss of the kanMX Pst I cleavage site. Pst I digestion of both of the rearranged plasmids produced a novel 1.5 kb band not present in the pWDH445 digestions. If only the Pst I cleavage site in kanMX3 had been altered in such a way as to prevent its cleavage, the appearance of a ˜3.5 kb band would be expected (combination of the 2.5 and 0.9 kb fragments). However, recombination between the direct repeat elements flanking the Kan^{r} gene in the kanMX3 module could result in the loss of a 2 kb portion of the 3.5 kb fragment. This may account for the 1.5 kb band detected in lanes 3 and 5.

### Two Out of Three Pvu I Cleavage Sites were Lost in Both Rearranged Plasmids

There are four cleavage sites for the Pvu I restriction endonuclease in pWDH445, 3 of which lie within the kanMX3 module. The four fragments expected were of the sizes ˜9, 1.2, 1.1, and 0.7 kb. The four plasmids were digested with Pvu I and the resulting fragments were fractionated on a 1% agarose gel by electrophoresis. The digested plasmids were loaded onto the gel with stock pWDH1445 in lane 1, the larger rearranged plasmid in lane 2, isolated pWDH445 in lane 3, and the smaller rearranged plasmid in lane 4. FIG. 9 shows a picture of the ethidium bromide stained Pvu I fragments. Both lanes 1 and 3 exhibit all four of the expected bands whilst lanes 2 and 4 reveal different band patterns. The smaller rearranged plasmid in lane 4 showed the large band, representing the bulk of the plasmid, and a band at 1.1 kb that all four plasmids exhibit. The 1.1 kb band results from cleavage by Pvu I in the Amp^{r} and Kan^{r} genes, suggesting that the Pvu I cleavage site at the 5′ end of kanMX3 was intact. However, neither rearranged plasmid shows bands representing the 1.2 and 0.7 kb kanMX3 internal fragments. This suggests that a loss of DNA within the kanMX3 module has occurred. The larger rearranged plasmid also showed 2 novel bands at 1 kb and ˜3.5 kb.

### Sac I Does not Liberate ˜2.1 kb of kanMX3 from the Rearranged Plasmids

There are 2 Sac I cleavage sites in pWDH445, one in each of the direct repeat sequences flanking Kan^{r} in kanMX3. Thus, 2 bands representing 2 fragments of ˜2.1 kb and ˜10 kb should be produced by Sac I digestion of pWDH445. The 2.1 kb fragment is released from kanMX3 whilst the larger fragment represents the remainder of the plasmid. The four plasmids were digested with Sac I restriction endonuclease and the resulting fragments were separated electrophoretically in a 1% agarose gel. FIG. 10 is a picture of the ethidium bromide stained gel with pWDH445 in lane 1, the larger rearranged plasmid in lane 2, isolated pWDH445 in lane 3, and the smaller rearranged plasmid in lane 4. Only lanes 1 and 3 exhibit the expected 2.1 kb fragment released from kanMX3, whilst lanes 2 and 4 both show only a single band representing uncut plasmid. In conjunction with the other evidence discussed above, this suggests that both rearranged plasmids have lost a portion of kanMX3 between and including the Sac I cleavage sites in the direct repeat sequences.

### Enzymic Removal of kanMX3 from pWDH445

The nature of the investigations described above is such that a considerable amount of time could have been consumed in attempting to precisely define the rearragement(s). Since a significant rearrangement was defined within the kanMX module, it was decided to re-create this modification by enzymically excising kanMX3. This would provide a means to test the hypothesis that the enhancement of brightness from cells bearing the rearranged plasmids was due to the loss of part of the kanMX3 module.

### Sac I Digestion of pWDH445 Followed by Re-Ligation

Stock pWDH445 was digested with Sac I to completion, in order to remove a ˜2.1 kb fragment from the kanMX3 module. A 3 μl aliquot of Sac I-digested pWDH445 was loaded onto a 1% agarose gel and the fragments separated by electrophoresis in order to check the success of the digestion (data not shown). After heat inactivation of the Sac I enzyme by incubation at 65° C. for 20 minutes, digested pWDH445 was then re-ligated. Ligations were performed at 3 different concentrations of digested plasmid DNA (undiluted, 1 in 10 fold dilution, and 1 in 100 fold dilution) by incubation with T4 DNA ligase at 16° C. overnight. Dilutions of digested DNA were used in order to promote intramolecular ligation events. A 10 μl aliquot of each ligation mix was used to transform E. coli and transformants were selected by overnight incubation at 37° C. on LB plates containing ampicillin. Ampicillin resistant transformants were replica-plated onto LB plates containing kanamycin. After overnight incubation on kanamycin plates, very few colonies were able to grow.

10 ampicillin resistant colonies were selected, including one transformant that was also kanamycin resistant, and incubated with shaking in LB broth containing ampicillin at 37° C. overnight. Plasmid preparations were made from these cultures using standard techniques and the isolated DNA digested with Xba I along with normal pWDH445. Fragments were separated by electrophoresis in a 1% agarose gel and the ethidium bromide stained gel is shown in FIG. 11. Lanes 1 and 13 show the bands representing the xba I fragments from pWDH445, though overloading makes the 6 kb fragment difficult to distinguish. Lane 7 contained a 500 bp ladder molecular weight marker with the largest band at 5 kb, demonstrating that the bands in lanes 1 and 13 were ˜6 kb. Lane 10 contained DNA from the kanamycin resistant colony and reveals the same 6 kb band as the controls in lanes 1 and 13. The remainder of the lanes contained DNA from kanamycin sensitive colonies and all exhibit a band at 6 kb, but also a second band at ˜4.5 kb. This is the same banding pattern as that seen for Xba I-digestion of the smaller rearranged plasmid (see lane 4, FIG. 6).

### The New Plasmid is Brighter than pWDH445 in Yeast

FF18984 cells were transformed with DNA from preparations of the new plasmid using standard methods. Transformants were selected for by incubation on SD plates lacking uracil and the auxotrophic markers were checked. 10 transformants were picked for assessment of brightness in response to 0.005% MMS. Incubation with MMS lasted 15 hours before measurement of fluorescence and scatter. The average brightness values determined for cells bearing the new plasmid (red bars) are compared with those from cells carrying pWDH445 (blue bars) in FIG. 12. The brightness values for the cells carrying the plasmid with part of kanMX3 enzymatically excised (FIG. 12) were comparable with those of cells bearing the spontaneously modified plasmid isolated from yeast. Untreated cells bearing the new plasmid were found to be 4 fold brighter than those conveying pWDH445, whilst the brightness increased 5 fold for cells treated with MMS. However, the induction ratio was reduced in the brighter cells due to the increase in the constitutive level of brightness.

### Summary

1. Transformation of FF18984 with pWDH445 generated some transformants with a 12 fold brighter constitutive signal and a 9 fold brighter MMS-induced signal than typical pWDH445-bearing FF18984 cells. The induction ratio was halved in the brighter cells.
2. Bright transformants were unable to grow on medium containing G418, suggesting an alteration within the kanMX3 module of pWDH4454.
3. Double restriction of plasmids isolated from the brighter transformants with BamH I and Asc I produced the same size bands as pWDH445, suggesting that the HO-RAD54 promoter-yEGFP module was unchanged. However, one rearranged plasmid was larger than pWDH445 whilst another was smaller.
4. Yeast cells (FF18984) re-transformed with rearranged plasmid were sensitive to G418, confirming that the change in brightness was due to the plasmid change, as opposed to a chromosomal mutation.
5. Untreated and MMS-induced re-transformed yeast cells were 3.3 fold and 4.6 fold brighter than pWDH445-carrying cells, respectively. However, there was little change in induction ratio between cells conveying pWDH445 or rearranged plasmid.
6. Digestion with Xba I corroborated the finding from BamH I-Asc I double digestion that one plasmid was larger and one smaller than pWDH445. The fragment altered by the rearrangements carries Amp^{r}, kanMX3, and yEGFP.
7. Sca I digestion suggested that no rearrangement had occurred in the region bearing HO, URA3, the 2 micron origin, and part of Amp^{r}. In conjunction with the findings from the BamH I-Asc I and Xba I digestions, rearrangement must have occurred between the unique Asc I cleavage site and the Sca I cleavage site in Amp^{r}, in a region incorporating the entire kanMX3 module.
8. The 2 bands formed by Pst I digestion if the Pst I site in kanMX3 is intact are not present for either of the rearranged plasmids.
9. 2 of 3 Pvu I cleavage sites in kanMX3 are not present in the rearranged plasmids suggesting loss of DNA within the kanMX3 module.
10. Sac I does not release the expected ˜2.1 kb kanMX3 fragment from the rearranged plasmids.
11. A ˜2.1 kb fragment of kanMX3 was cleaved from pWDH445 using Sac I before religation of the Sac I sticky ends. Yeast cells transformed with the new plasmid were as bright as the yeast cells bearing spontaneously rearranged plasmids.

### Discussion

Rearrangements of pWDH445 (to form recombinant vectors according to the first aspect of the invention) occurred, probably in a transformation-dependent manner given that transformation is known to be a mutagenic process, generating brighter than normal transformants. These bright transformants were found to be sensitive to growth on medium containing 200 μgml⁻¹ geneticin, despite pWDH445 bearing a kanMX module incorporating the gene encoding the aminoglycoside phosphotransferase that confers resistance to geneticin on yeast cells. This suggested that mutation or loss of the kanMX module had occurred, leaving the cells sensitive to geneticin. It was anticipated that kanMX had probably been lost from the plasmid, since a deletion event would reduce the size of the plasmid, increasing its stability and hence copy number.

However, after isolation of plasmid DNA from the bright transformants and digestion to release the HO-RAD54promoter-yEGFP cassette, it was clear from the bands representing the DNA fragments separated by gel electrophoresis that whilst one bright transformant carried a smaller reporter plasmid, another conveyed a larger derivative of pWDH445. This ruled out the hypothesis that the enhanced reporter output was a plasmid size-related phenomenon and thus, demanded more detailed investigation. The outcome of further restriction analysis of the rearranged plasmids revealed that kanMX was lost in both the smaller and larger plasmids, and the larger plasmid gained several kb of DNA.

The smaller plasmid was probably generated by recombination between the direct repeats that flank the kan^{r} gene and promoter sequences within the kanMX module. The kanMX fragment released by Sac I digestion is ˜2.1 kb which is approximately the size of fragment lost by the small plasmid.

It has been previously shown that bacterial sequences borne by shuttle vectors can be toxic to heterologous host organisms and that such sequences unnecessary for maintenance in S. cerevisiae can be automatically eliminated by recombination events. Since all pWDH445 transformants were primarily selected for their ability to grow on SD medium lacking uracil, the kanMX module was unnecessary for plasmid maintenance.

Seemingly the enhancement of reporter brightness is not related to a reduction in size and hence increase in copy number, since one of the rearranged plasmids was shown to have increased in size. This suggests that the constitutive expression of the aminoglycoside phosphotransferase driven by the promoter from the translation elongation factor 1α (AgTEF) from the filamentous fungus Ashbya gossipyii, affects the expression of yEGFP from the RAD54 promoter in some way. The kanMX module is immediately downstream of the HO-RAD54promoter-yEGFP cassette with only a direct repeat sequence (465 bp) separating yEGFP from AgTEF. While we do not wish to be bound by any hypothesis, it is conceivable that the omnipresent (due to the constitutive expression) transcription complex for the aminoglycoside phosphotransferase interferes with termination of yEGFP transcription, resulting in imperfect transcripts and hence nascent yEGFP polypeptide that is incapable of legitimate folding to produce mature GFP. Loss of kanMX would remove the interference and permit a greater proportion of yEGFP to fold correctly and hence fluoresce, in both constitutive and induced reporter states.

Interference of efficient transcription of yEGFP might result from the conformation of the plasmid. The size and sequence of pWDH445 bearing kanMX might be such that the plasmid assumes a conformation restricting access of components of the transcriptional complex to the RAD54 promoter. However, loss of kanMX regardless of the mechanism could result in alteration of the conformation, due to the change in size and sequence, leading to greater accessibility of the RAD54 promoter. In this case, it must be that pWDH445 is of the critical size allowing it to assume the restrictive conformation, and hence any change in size, either increase or reduction, alters the conformation sufficiently.

An alternative explanation for the enhanced brightness in the absence of kanMX is the greater availability of general transcription factors. Since the aminoglycoside phosphotransferase encoded within kanMX is constitutively expressed, it must have a constant requirement for general transcription factors. Thus, the availability of such factors for transcription from the RAD54 promoter is reduced in the presence of kanMX, and removal of this module eliminates the constraints on the reporter cassette. This is most likely only a “local” deficiency in the volume occupied by the plasmid, and might reflect a higher affinity for the transcription factors in the AgTEF promoter.

Finally, kanMX related interference with reporter expression might occur post-transcriptionally. Both yEGFP and the aminoglycoside phosphotransferase are heterologous proteins since GFP originates from Aequorea victoria and Kan^{r} from the E. coli transposon Tn903. It is possible that the 2 foreign proteins interact, preventing efficient protein folding and maturation of yEGFP. Loss of kanMX would result in loss of the illegitimate interaction, increasing the proportion of fluorescent yEGFP.

The mechanism by which kanMX is lost and reporter output is enhanced is essentially irrelevant to the commercial development of the reporter (as long as the response remains predictable), since a brighter reporter is obviously beneficial. The kanMX module was employed as a convenient selectable marker for research purposes, but stringent rules governing the release of genes conferring antibiotic resistance would require its displacement for commercialisation.

If the kanMX effect seen with pWDH445 is purely a plasmid-related phenomenon then the reporter cassette could be re-cloned into a different plasmid backbone, in order to determine if the effect is specific to pWDH445. Replacement of kanMX with a different gene conferring antibiotic resistance, such as hygromycin B (hph; hphMX), nourseothricin (nat; natMX), and bialaphos (pat; patMX), would resolve whether the effect is specific to kanMX on pWDH445. An intergenic region could be inserted between yEGFP and kanMX on pWDH445 to determine whether the effect is caused by the proximity of the AgTEF promoter of kanMX to the termination codon of yEGFP. Assessment of these new vectors would be by constitutive and induced brightness values when borne by the FF18984 strain. Bright transformants have been isolated from other yeast strains though plasmid DNA has not been isolated and assessed in the same way as from bright FF18984 cells. However, it is anticipated that similar rearrangements would be detected in plasmid isolated from alternative strains, which would discount a strain-specific effect.

### EXAMPLE 2

A yeast DNA repair reporter assay termed the GreenScreen® assay (GSA) is described. This assay represents a preferred method of detecting for the presence of an agent that causes DNA damage according to the third aspect of the invention.

We describe the simple, robust assay protocol, the development and a validation study; results of which indicate that compounds giving positive responses have a high likelihood (96%) of corresponding positive responses in one or more of the regulatory tests (Ames, Micronucleus and Mouse Lymphoma Tests or carcinogenicity bioassays). The false positive rate is extremely low. The endpoint of the test reflects the typically eukaryotic chromosomes and DNA metabolising enzymes of Saccharomyces cerevisiae. The capacity for metabolic activation (MA) is limited compared to the mammalian liver or its extracts, but the assay does detect a subset of compounds that would require MA in existing genotoxicity tests. The GSA detects a different spectrum of compounds to bacterial genotoxicity assays and thus, together with an in silico Structure Activity Relationship (SAR) screen, and possibly a high throughput bacterial screen, would provide an effective preview of the regulatory battery of genotoxicity tests.

### Introduction

The introduction of high-throughput bacterial screens such as the SOS umu and SOS lux tests have demonstrated that it is possible to apply genotoxicity testing to the small quantities of test chemical available at the early stages of the drug discovery process. This type of assay may also have an important role in such diverse disciplines as the environmental monitoring and food industries and military field-testing. Although these bacterial assays have proved their effectiveness, they have some disadvantages; especially because they use non-eukaryotic cells and hence will not detect genotoxins that interact with eukaryotic-specific targets.

The inventors have previously reported the construction and preliminary validation of a yeast-based genotoxicity test system, RAD54-GFP (see WO 98/44149). Induction of the RAD54 promoter results in the production of the extremely stable Green Fluorescent Protein (GFP), which is fluorescent in the green spectrum when illuminated by blue light. It appears that any agent able to cause mutation in yeast will lead to RAD54 induction. The specificity to DNA damage has been confirmed by studying the global transcriptional response to DNA-damaging agents using DNA microarrays. It was found that RAD54 did not respond to other non-genotoxic stresses, such as heat shock, oxidative stress, reductive stress, osmotic shock or amino acid starvation.

RAD54 encodes a structural element of the homologous recombinational (HR) repair pathway, but responds to a broad spectrum of genotoxins, suggesting that the yeast DNA damage sensing pathways activate the HR pathway as a default for failure of the other repair pathways. This is supported by several lines of genetic evidence. For example the loss of the non-homologous end-joining (NHEJ) pathway in yeast is undetectable unless the RAD52 (HR) pathway is also ablated. Furthermore, it has been demonstrated that induction of RAD54 by methyl methanesulfonate (MMS) is insensitive to mutations in the ‘classical’ DNA-damage sensing pathway controlled by the RAD9 and DDC1 checkpoints. This suggests that there are additional DNA damage sensing pathways leading to RAD54 activation and that this may account for its broad response profile. The HR DNA-repair process is well characterised and is apparently highly conserved within all eukaryotic organisms. Expression of the human homologue hRAD54 in yeast largely suppresses the phenotypes resulting from deletion of the native copy. Increased expression of RAD54 in yeast as a result of exposure to genotoxins is therefore likely to be predictive of the increased DNA repair activity in mammalian cells.

Here we describe a protocol suited to the use of robotic liquid handling systems and present simple data handling protocols that provide clear graphical output. We report the results of an assay validation program in which 102 compounds with diverse genotoxic potential have been tested.

### Materials and Methods

### Strains & Plasmids

The yeast strains, plasmids and growth media (F1) used in this study have been described previously (Walmsley et al., 1997) and the use of Green Fluorescent Protein (GFP) as a reporter for the DNA damage-induced gene RAD54 from Saccharomyces cerevisiae, has also been described (Billinton, et al., 1998). The Saccharomyces cerevisiae strain FF18984 (MATa, leu2-3,112 ura3-52 lys2-1 his7-1) was obtained from Francis Fabre (French Atomic Energy Commission (CEA), Fontenay-aux-Roses, France).

The reporter strain (GenT01) is FF18984 containing a replicative plasmid (pGen001 shown in FIG. 15) containing the entire upstream non-coding DNA sequence of the RAD54 gene fused to the yeast-enhanced Aequorea victoria GFP gene. The control strain (GenC01) is FF18984 containing an identical plasmid except that 2 base pairs have been removed at the start of the GFP gene, such that no GFP is made.

### Microplate Preparation

Assays were carried out in 96 well, black, clear-bottomed microplates (Matrix ScreenMates, Cat. No. 4929, Apogent Discoveries, USA). A number of alternative microplates were assessed, though the variable background absorbance and fluorescence both within and between plates from individual manufacturers were generally unacceptable, leading to the conclusion that only Matrix or Corning (BV, Netherlands: Cat. No. 3651) plates were appropriate for this assay. The assays were performed using a liquid handling robot (MicroLabS single probe, Hamilton GB Ltd., Birmingham. UK) in a protocol designed to set up 4 compounds per test on a single 96 well microplate in 30 minutes. Results for a subset of compounds have been reproduced using a Genesis 8-probe robot (Tecan UK Ltd. Theale. UK), which can set up a similar microplate in less than 5 minutes. Microplates can also be filled rapidly and effectively using a multi-channel pipette.

A microplate version of the assay has been previously reported (Afanassiev et al., 2000), but different microplate layouts and controls have been used for this study, so more detail is presented here. The following standard protocol was followed. A 1 mM stock of the test chemical was prepared in 2% v/v aqueous DMSO, and used to make 2 identical dilution series across the microplate and a ‘control’ (see below). To achieve this, 150 microlitres of the test chemical solution were put into 2 microplate wells. Each sample was serially diluted by transferring 75 microlitres into 75 microlitres of 2% DMSO, mixing, and then taking 75 microlitres out and into the next well. This produced 9 serial dilutions of 75 microlitres each.

Controls were added as follows:

a. Compound alone, to provide information on compound absorbance/fluorescence
b. Yeast cultures diluted with 2% DMSO alone, to give a measure of maximum proliferative potential
c. MMS as a genotoxicity control: ‘high’=0.00125% v/v, ‘low’=0.0001875% v/v
d. Methanol as a cytotoxicity control: ‘high’=3.5% v/v, ‘low’=1.5% v/v.
e. Growth medium alone, to confirm sterility/lack of contamination

Stationary phase cultures of GenT01 and GenC01 were diluted to an optical density (OD_{600 nm})=0.2 in double strength F1 medium (Billinton et al., 1998). 75 microlitres of the yeast suspension were added to each well of the diluted chemical: GenT01 to one series and GenC01 to the second, and to appropriate standards and controls. After the plates were filled, they were sealed using either a gas permeable membrane (Breath-easy, Diversified Biotech, USA) or a plastic lid, and then incubated without shaking, overnight at 25° C.

### Compounds Chosen for the Study

In order to compile a list of chemicals unbiased by the inventors' laboratory, a number of pharmaceutical companies and contract research organisations were asked to suggest compounds for which results would be of interest in assessing a new test. All compounds were sourced at analytical purity where available (Sigma, Aldrich, Fluka, BDH, Avocado).

### Data Collection and Handling

Following overnight incubation, fluorescence and absorbance data were collected from the microplates. Two different microplate readers which combine fluorescence and absorbance functionality have been used, and comparable data were obtained. These were a Tecan Ultra-384 (Tecan UK Ltd.): excitation 485 nm/emission 535 nm with an additional dichroic mirror (reflectance 320 nm-500 nm, transmission 520 nm-800 nm), and a BMG PolarStar (BMG Labtechnologies, Germany): excitation 485 nm/emission 520 nm. Absorbance was measured through a 620 nm filter in both instruments. The data were transported into a Microsoft Excel spreadsheet, and converted to graphical data (see typical data in FIG. 18). Data processing is minimal: absorbance data give an indication of reduction in proliferative potential and these data were normalised to the untreated control (=100% growth). Fluorescence data were divided by absorbance data to give ‘brightness units’, the measure of average GFP induction per cell. These data were normalised to the untreated control (=1). In this way, one can distinguish between a small number of highly fluorescent cells and a large number of weakly fluorescent cells. In order to correct for induced cellular autofluorescence and intrinsic compound fluorescence, the brightness values for the GenC01 strain were subtracted from those of GenT01. This makes visual assessment of the data more reliable. All the data were checked with and without this correction, and the decision (see below), on whether or not a compound was classified as being genotoxic, was not affected.

### Decision Thresholds

It is useful to have clear definitions of positive and negative results from routine assays and such definitions have been derived, taking into account the maximum noise in the system and data from chemicals where there is a clear consensus on genotoxicity and mechanism of action. Naturally it is also possible for users to inspect the numerical and graphical data and draw their own conclusions. For example an upward trend in genotoxicity data that did not cross the threshold might still distinguish two compounds. The decision thresholds were set as follows:

The cytotoxicity threshold is set at 80% of the cell density reached by the untreated control cells. This is greater than 3 times the standard deviation of the background. A positive cytotoxicity result (+) is concluded if 1 or 2 compound dilutions produce a final cell density lower than the 80% threshold. A strong positive cytotoxicity result positive (++) is concluded when either (i) three or more compound dilutions produce a final cell density lower than the 80% threshold or (ii) at least one compound dilution produces a final cell density lower than a 50% threshold. A negative result (−) is concluded when no compound dilutions produce a final cell density lower than the 80% threshold. The lowest effective concentration (LEC) is the lowest test compound concentration that produces a final cell density below the 80% threshold.

The compound absorbance control allows a warning to be generated if a test compound is significantly absorbing. If the ratio of the absorbance of the compound control well to a well filled with diluent alone is >2, there is a risk of interference with interpretation. The cytotoxicity controls indicate that the yeast is behaving normally. The ‘high’ methanol standard should reduce the final cell density to below the 80% threshold, and should be a lower value than the ‘low’ standard.

The genotoxic threshold is set at a relative GFP induction of 1.3 (i.e. a 30% increase). This is greater than 3 times the standard deviation of the background. A positive genotoxicity result (+) is concluded if 1 or 2 compound dilutions produce a relative GFP induction greater than the 1.3 threshold. A strong positive genotoxicity result (++) is concluded if either (i) three or more compound dilutions produce a relative GFP induction greater than the 1.3 threshold or (ii) at least one compound dilution produces a relative GFP induction greater than a 1.6 threshold. A negative genotoxicity result (−) is concluded where no compound dilutions produce a relative GFP induction greater than the 1.3 threshold. The LEC is the lowest test compound concentration that produces a relative GFP induction greater than the 1.3 threshold. The genotoxic controls demonstrate that the strains are responding normally to DNA damage. The ‘high’ MMS standard must produce a fluorescence induction >2, and be a greater value than the ‘low’ MMS standard. Anomalous brightness data is generated when the toxicity leads to a final cell density less than 30% that of the blank. Genotoxicity data is not calculated above this toxicity threshold. Compounds that tested negative for genotoxicity, were re-tested up to 10 mM, or to the limit of solubility or cytotoxicity.

The compound fluorescence control allows a warning to be generated when a compound is highly auto-fluorescent. If the ratio of the fluorescence of the compound control well to a diluent filled well is >5, there is a risk of interference with interpretation. In these cases (four in this study), fluorescence polarisation can be used to distinguish GFP from other sources of fluorescence (Knight et al., 2000, 2002). Both the Tecan and BMG instruments have this facility. Occasionally, compounds though not fluorescent themselves, induce cellular auto-fluorescence. This is apparent from rising brightness in the control (GenC01) strain in the absence of fluorescence from the chemical-only control. The routine subtraction of GenC01 from GenT01 data removes this interference from the data.

### Comparisons

Published results from other genotoxicity tests and cancer studies have been tabulated. They include Ames test, Mouse Lymphoma Assay (MLA), in vitro and in vivo cytogenetics (chromosome aberration assays) and rodent Micronucleus Test (MNT). The data have been collected both from the peer-reviewed literature and from freely available internet resources. These include CCRIS (Chemical Carcinogenesis Research Information System: http://toxnet.nlm.nih.gov), NTP (National Toxiciology Program Reports: http://ntp-server.niehs.nih.gov), IARC (International Agency for Research on Cancer: http://www.iarc.fr), NIOHS (National Institute for Occupational Health and Safety: http://www.cdc.gov/niosh/homepage.html) and the USEPA (Environmental Protection Agency: http://www.epa.gov/iris/search.htm).

There are several compounds where there are conflicting data from different sources (recorded as +/−). However, where comparisons have been drawn these are recorded as positive (+). The only exception to this is where NTP has more recently suggested a definitive designation. It is not intended in this paper to reclassify chemicals on the basis of other test results and the authors apologize for any inadvertent gaps or confounding decisions.

### Results

The results of the validation study incorporating 102 chemicals are shown in full in FIG. 20. For completeness it includes some compounds tested at very high concentrations where positive genotoxicity results have been concluded from higher concentrations in other studies.

In comparison with the other test data, it is clear that the sensitivity of the test is high. 55 compounds were positive for genotoxicity in GSA, 50 of which had comparable data from other genotoxicity and cancer studies. Excluding the cancer results, 47/50 GSA positive compounds were positive in either Ames, MLA, MNT or chromosome aberration. Considering just the mammalian test data (i.e. excluding Ames data) 40 of 44 GSA positive compounds (91%) have positive data from MLA, MNT or chromosome aberration studies. However, if all data is included (i.e. mammalian, Ames and the 2 additional compounds with only positive cancer data, (3-amino-1,2,4-triazole and sulfamethoxazole), the sensitivity of the test to genetic damage increases to 96% (48/50). A second important statistic from the whole study is that there were only two compounds positive in GSA for which there was no corresponding positive data from other tests. These were tritolyl phosphate and cimetidine.

TABLE 12
Suggested maximum tolerable solvent concentrations.

| | | Maximum Concentration (% |
|---|---|---|
| Test Compound | CAS No. | v/v) |
| Carbon tetrachloride | 56-23-5 | 2 |
| 1-Chloro-2-propanol | 127-00-4 | 2 |
| Dimethylsulfoxide | 67-68-5 | 5 |
| Ethanol | 64-17-5 | 2 |
| Methanol | 67-56-1 | 2 |
| Tetrahydrofuran | 109-99-9 | 2 |

Table 12 lists chemicals/solvents that are often used with test samples, to promote dissolution etc. All of these compounds are toxic at higher concentrations due to osmotic effects and/or effects on membranes, and interfere with data interpretation. If these chemicals are used to dissolve compound stock solutions, they should also be included in the diluent used to make the compound dilution series. The routine use of DMSO in this study revealed its predictable interference with certain compounds. For example, cisplatin was only positive when retested using water in place of 2% DMSO as the diluent. Interference of DMSO, a free radical scavenger, in the recombinogenic properties of several oxidising agents has previously been reported.

| | | MNT | MNT | | Chrom. | SOS | |
|---|---|---|---|---|---|---|---|
| Term | MLA | in vivo | in vitro | Ames | Ab. | umuC | Carc. |
| Number of | 56 | 52 | 30 | 93 | 36 | 43 | 69 |
| compounds | | | | | | | |
| (N) | | | | | | | |
| Prevalence/ | 88 | 69 | 73 | 51 | 83 | 58 | 77 |
| % | | | | | | | |
| Predictive | 87 | 67 | 83 | 64 | 95 | 71 | 89 |
| Value/% | | | | | | | |
| Sensitivity/% | 53 | 46 | 63 | 68 | 50 | 77 | 62 |
| Specificity/ | 43 | 53 | 67 | 61 | 83 | 53 | 76 |
| % | | | | | | | |

Several different indices have been used to assess the utility of new tests, and the relative value of these has been discussed elsewhere (Cooper et al., 1979). The appropriate terms and their definitions are taken from Table 1 in the latter paper and are paraphrased as follows.

The test with GSA can have a positive outcome, for which there may be either a positive result (‘a’) or a negative result (‘b’) from another test (for example MLA). The total number of positives for GSA is thus ‘a+b’. Similarly, the test with GSA can have a negative outcome, for which there might be either a positive (‘c’) or a negative (‘d’) result form the second test. The total number of negatives for GSA is thus ‘c+d’. It follows that the total number of positive results from the second test is ‘a+c’ and the total number of negative results from the second test in ‘b+d’. similarly the total number of compounds for which there is data for both tests ‘n’ is ‘a+b+c+d’. The following terms were calculated from these data:

Sensitivity, a/(a+c); specificity, d/(b+d); predictive value, a/(a+b); prevalence, (a+c)/n

FIG. 21 contains the subset of data for which there are Ames data, and highlights the distinctively different endpoints for GSA and bacterial genotoxicity results. There are 93 compounds in this group. 18 compounds that are positive in GSA are negative for the Ames test (with or without S9), 17 of which are positive in animal carcinogenesis or mammalian cell tests. 6 of 31 GSA positives that are Ames positive, required metabolic activation for the Ames result. 15 Ames positive compounds were negative in GSA, of which 6 are only Ames positive with metabolic activation.

### Discussion

The induction of the yeast RAD54 gene has been monitored using a promoter-GFP fusion. In a previous study (Afanassiev et al., 2000), a small sample of chemicals (12) was selected to provide an indication of the range of DNA damaging agents that could be detected. That study used a slightly different experimental protocol, though the results demonstrated a good coverage of DNA damaging agents as well as enzyme inhibitors including DNA polymerase (aphidicolin) and topoisomerase 1 (camptothecin) inhibitors.

In selecting compounds for this extended study it was decided that a very restricted list of compounds with expected results (unambiguous mutagens and non-mutagens) would not give a very broad definition of the endpoint. Broader selections of the ‘obvious’ in other studies have previously led to dispute about what the expected result should be. Thus the results presented here come from a collection of test compounds not selected by the authors. Naturally, it includes many obvious mutagens and non-mutagens; it also includes compounds with either conflicting genotoxicity data from other sources, or very little other data. The latter were clearly suggested to the authors to give a better idea of the test endpoint.

This broader study of 102 compounds (FIG. 20) confirms that the RAD54 gene is induced by a variety of DNA damage lesions caused by direct-acting agents. These include examples of base alteration (MMS, ethidium bromide), clastogenicity (chromosome breakage: bleomycin, catechol, 1,2-dimethylhydrazine, methyl viologen, methapyraline, phthalic acid bis(2-ethylhexyl) ester, phleomycin), and cross-linking (mitomycin C, cisplatin). Amongst the oxidising agents, cumene hydroperoxide is a notable negative. It is interesting that the photomutagen psoralen was a GSA positive as the test was only exposed to normal laboratory lighting. The reporter also responds to a variety of genotoxins that may not act directly on DNA. These include compounds that target topoisomerases (etoposide, ellipticine) as well as anti-mitotic, spindle targeting compounds (aneugens) such as colchicine and econazole. The latter results are interesting because they suggest that some aneugens can also lead indirectly to DNA damage. Importantly for a proposed screening test, only two positive results were unique to yeast, tritolyl phosphate and cimetidine. It would be interesting to discover the chemistry behind these results though it is intriguing that the latter (cyano-methyl-guanidine) is chemically related to methyl-nitro-nitrosoguanidine (MNNG), a well-known mutagen.

The group of other carcinogens not detectable by Ames (including safrole, urethane, aniline and thiourea) is interesting and here the GSA result confirms similar observations using the yeast DEL assay.

The spectrum of metabolic enzymes expressed by yeast is less complex than that of mammalian cells. There are, for example, the CYP (cytochrome P450) type enzymes (ERG5/CYP61; DIT2/CYP56; P450_{14-DM}/ERG11/CYP51; the P450 oxido-reductase NCP1/CPR1 and glutathione-S-transferases GTT1 and GTT2. This proficiency is reflected by the identification of 6 compounds producing positive data with GSA that require S9 for an Ames positive result (Neutral Red, 2-amino-4-nitrophenol, proflavin hemisulfate, ethidium bromide, benzo[a]pyrene, 1-naphthylamine). S9 was not used in any of the GSA tests reported in this paper.

The metabolic capability of yeast contrasts to the enhanced activities in S9 required for the activation of most promutagens, and explains the most obvious group of compounds not detected by GSA: primary aromatic amines and aromatic amides. 9 such compounds were tested. Two were positive in GSA: 2-amino-4-nitrophenol and 1-naphthylamine. The others were negative in GSA and positive in other in vitro tests: 2-acetamidofluorene, 9-aminoacridine, 2-aminoanthracene, 4,4-oxydianiline, o-anisidine, aniline and 4-aminophenol. The last two of these are negative with and without S9 in Ames. The aromatic amine motif itself is recognised by SAR programmes so whilst missed by GSA, such compounds would be unlikely new drug candidates. It has been demonstrated that the metabolic competence of yeast strains could be enhanced by the expression of human cytochrome P450 genes. In preliminary experiments using plasmids from the Sengstag group in the yeast strains used in this study, the RAD54-GFP reporter has also increased its spectrum of response. A full assessment of these strains is underway (including an assessment of an S9 protocol) and these will be reported when complete.

To assess the relative compound requirement of the GSA when compared to bacterial screening tests, the Lowest Effective Concentrations (LEC) of a small sample of the validation chemical set were compared in Table 13.

This comparison indicates that the GSA is of comparable sensitivity to the SOS umuC chromotest and the SOS lux bioluminescence assay (based on a very limited dataset). Therefore the total compound requirement of the GSA is comparable to other commonly used bacterial short-term screens.

The GSA has a simple and fixed microplate protocol with clear decision-making thresholds. Together these properties were conceived to give a high throughput capability and to minimise the generation of conflicting test data. These are important requirements of any useful screen. The compounds tested in this screen provide sufficient data to reveal that a positive result with GSA provides a relevant warning of positive results in the regulatory battery of tests, and in the present form provides complementary data to bacterial tests—each providing significantly non-overlapping endpoints. It will be interesting to see how the definition of the endpoint develops as more data become available.

### REFERENCES

Afanassiev, V., Sefton, M., Anantachaiyong, T., Barker, M. G., Walmsley, R. M., and Wolfl, S. (2000). Application of yeast cells transformed with GFP expression constructs containing the RAD54 or RNR2 promoter as a test for the genotoxic potential of chemical substances. Mutat. Res., 464, 297-308.
Billinton, N., Barker, M. G., Michel, C. E., Knight, A. W., Heyer, W.-D., Goddard, N. J., Fielden, P. R. and Walmsley, R. M. (1998). Development of a green fluorescent protein reporter for a yeast genotoxicity biosensor. Biosens. Bioelectron., 13, 831-838.
Cesareni, G. & Murray, J. A. H. (1987) in Genetic Engineering: Principals and Methods, eds. Setlow, J. K. & Hollaender, A. (Plenum, New York), Vol. 9, pp. 135-154.
Sikorski R S, Hieter P. A system of shuttle vectors and yeast host strains designed for efficient manipulation of DNA in Saccharomyces cerevisiae. Genetics 122: 19-27, 1989.
Wahmsley, R. M., Billinton, N. and Heyer W.-D. (1997). Green fluorescent protein as a reporter for the DNA damage-induced gene RAD54 in Saccharomyces cerevisiae. Yeast, 13, 1535-1545.

TABLE 13
A comparison of LEC data from GSA, umuC and lux assays.

| | | | SOS UMUC | SOS LUX |
|---|---|---|---|---|
| | | | ASSAY | ASSAY |
| COMPOUND | S9 | GSA (M) | (M) | (M) |
| BENZO[A]PYRENE | + | 1.0 × 10⁻⁴ | NT | 7.9 × 10⁻⁷ |
| CISPLATIN | − | 6.2 × 10⁻⁵ | 9.0 × 10⁻⁷ | NT |
| HYDROGEN | − | 2.0 × 10⁻³ | NT | 5.9 × 10⁻⁵ |
| PEROXIDE | | | | |
| METHYL | − | 9.3 × 10⁻⁶ | 1.5 × 10⁻⁴ | 7.3 × 10⁻⁵ |
| METHANESULFONATE | | | | |
| MITOMYCIN C | − | 6.0 × 10⁻⁴ | 1.0 × 10⁻⁸ | 4.7 × 10⁻⁸ |
| MNNG | − | 1.7 × 10⁻⁶ | 6.0 × 10⁻⁶ | NT |
| NALIDIXIC ACID | − | 1.6 × 10⁻⁶ | 2.4 × 10⁻⁶ | 6.9 × 10⁻⁷ |
| SODIUM AZIDE | − | 1.0 × 10⁻⁵ | 5.4 × 10⁻³ | — |

KEY
M = Molar concentration
NT = Not tested
MNNG = N-Methyl-N′-nitro-N-nitrosoguanidine

### EXAMPLE 3

This example provides a preferred protocol for carrying out the method of the invention using a plate assay procedure.

### 1) Preparing the Starter Cultures.

2 different strains are currently used per test: GenC01 (control strain) and GenT01 (the test strain). The cultures are inoculated and incubated in an orbital shaker at 200 rpm and 30° C. for three days (72 hours), after which the cells will be in ‘stationary phase’. Culture flasks are then stored in a refrigerator (+4° C.) and can be used for tests over a period of up to seven days.

Each starter culture is prepared as follows:

Into a sterile 250 ml conical flask place—
10 ml sterile water
10 ml 2× F1 media (See Appendix 1)
1×250 μl colour coded aliquot of frozen cells

Seal flask with foam bung (taped to the flask) before incubating.

### 2) Preparing the Test Compounds.

Standard solutions of the test compounds should be freshly prepared shortly before each test run. Unless a specific test concentration is required, the recommended top concentration of a test compound is 1 mM in 4% DMSO v/v in water. It is necessary to ensure that the final concentration of test compound is made up in the same diluent as that supplied to the robot for serial dilutions. The diluent is 4% DMSO v/v in water by default but can be substituted for water alone.

1 ml of test compound solution is required per ‘test run’ and there is sufficient capacity on a 96-well plate for 4 test runs. Therefore for each plate prepare 2 tubes each containing 1 ml of the respective test compound, label them, cover with black caps and leave in a fume cupboard until required. For reference record the details of the test compound, its concentration, dilution calculation and the diluent used.

### 3) Preparing the Control Compounds.

The control compounds are prepared in 4% DMSO v/v in water to the following concentrations:
MMS High=0.002500% v/v*
MMS Low=0.000375% v/v*
Methanol High=7% v/v*
Methanol Low=3% v/v*
200 μl of each control chemical is required per plate (this includes a volume required to allow safe automated pipetting distance). Hence for a set of 4 plates, make up at least 800 μl of each control.
By default all concentrations are halved when plated out, as a sample volume of 75 μl is combined with 75 μl of yeast culture producing a 50:50 dilution.

MMS and Methanol prepared from 100% stock solution with a diluent of 4% DMSO/water (v/v).

MMS:
5 μl 100% MMS+995 μl diluent=0.5% MMS Solution
Standard 1 (High): 20 μl 0.5% AMS+3980 μl diluent=0.002500%

MMS
Standard 2 (Low): 450 μl 0.0025% MMS+2550 μl diluent=0.000375%

MMS

Methanol:
Standard 1 (High): 280 μl+3720 μl diluent=7% Methanol
Standard 2 (Low): 120 μl+3880 μl diluent=3% Methanol

### 4) Preparing the Cells.

Measure the optical density (at 600 nm) of the ‘stationary phase’ GenT01 and GenC01 starter cultures using a 1 ml disposable cuvette with a 1 cm path length and water as a comparison blank. Due to the high density of the starter cultures, dilute 20 fold (950 μl sterile water+50 μl cell culture) to enable measurement in the linear range. Calculate the OD of the undiluted cultures (Y) using the equation below:

OD of the starter culture(Y)=Measured OD×20
Note: Once in cold storage (+4° C.) the cell density does not change significantly over the course of a week, and do not need to be re-measured before each day's use.

Prepare a fresh suspension of both GenT01 and GenC01 cells in 2×F1 media, at an initial OD of 0.2 units/ml. 4 ml of GenC01 and GenT01 cell suspensions are required per plate (3.3 ml plus dead volume). Therefore for 4 microplates the total volume of fresh cell suspension required is 16 ml for GenC01 and 16 ml for GenT01.

Use the following equation to calculate, V, the volume of starter culture required to prepare the new cell suspension:

$$V = \frac{0.2 \times volume of fresh culture required ( ml )}{Y}$$

Y=Optical density (O.D) of starter culture

Transfer V ml of stock cell culture into a sterile 5 ml glass test tube containing 4 mls of 2×F1 media.

Therefore for 4 microplates 16 ml of GenC01 is split into 4×4 ml aliquots in (blue capped) test tubes, and 16 ml of GenT01 is split into 4×4 ml aliquots (red capped) test tubes. For a lower number of plates scale down accordingly.

### 5) Preparing Additional Tubes and Solutions.

Place 3.5 ml of 90% ethanol in a single tube.

Place 3.5 ml of “media only” (1×F1) in a single tube.

Place approx. 900 ml of 10% ‘Chloros’ in a 1 L container as a disinfectant, and toxic chemical neutralising, solution into which caps and spent test tubes can be sterilised before washing, autoclaving and recycling.

### 6) Set-Up for the Pipetting Robot and Deck Layout.

Switching on and Software Set-Up
i. SWITCH ON the equipment in the following order.
   The fume hood.
   Computer.
   MVP (Diluent Selector Unit) at mains plug.
   The robot using the switch on right hand side.
   load up ‘Microlab Sampler Software’.
If the robot has been left filled with anything other than sterile water, purge the system with sterile water using a wash program before use. The following methods are available. To run use File/Open the click “Run Method”, “Start” and follow the on-screen instructions.

| | |
|---|---|
| Wash Needle/ | Washes 10 ml of a solution connected to MVP |
| System.Ame | position 1 into a waste container placed in the |
| | ‘Bleach wash’ position. |
| Wash Station/ | Washes the needle and “wash station” and all |
| Tubing.Ame | associated pump tubing for cycles of 10 s, from |
| | the “clean wash” reservoir to waste. |

ii. The control program reads in the settings for the chosen diluent, sample volume, yeast volume and the volume carried over in each serial dilution for each plate. The default values are as follows.

| PLATE | DILUENT | SMPVOL | YEASTVOL | DILVOL1 | DILVOL2 | DILVOL3 | DILVOL4 |
|---|---|---|---|---|---|---|---|
| 1, | 1, | 75, | 75, | 75, | 75, | 75, | 75 |
| 2, | 1, | 75, | 75, | 75, | 75, | 75, | 75 |
| 3, | 1, | 75, | 75, | 75, | 75, | 75, | 75 |
| 4, | 1, | 75, | 75, | 75, | 75, | 75, | 75 |

To alter the values or check the current configuration open the file C:/Program Files/Hamilton/Microlab Sampler/Methods/4COMPOUND.lay in Notepad. Make the alteration then select File/Exit and only when prompted with “The text in the file has changed. Do you want to save the changes?” select Yes to resave the file.
iii. In the Microlab Sampler software select File I Open and open 4COMPOUND.lay
iv. Select “Run Method”.

Loading the Robot Deck and Plating Out
i. Before commencing any method carefully clean the outside of the needle by hand with ethanol using a soft cloth.
ii. Load the required number of Matrix micro-plates onto the robot deck in the positions as shown on the screen.
iii. Load the GREEN RACK with the appropriate tubes containing yeast cultures, samples, standards, 90% ethanol, plate blank and an addition one for waste volumes of the test compounds according to the loading sequence (see FIG. 21).
iv. Place the appropriate diluent solutions in position to the right hand side of the deck with tube lines connected to the corresponding MVP selector positions. (Position 1 by default).
v. Place the ‘clean wash’ tube in a container of sterilised water (approx. 300 ml per plate).

vi. Ensure the ‘dirty water’ receptacle has sufficient capacity to collect the wash solution supplied.
vii. Remove all tube caps and place in a suitable disinfectant/compound neutralising solution (i.e. 10% Chloros).
viii. Select START in the ‘Run Method’ software.
ix. The method will now run taking approximately 30 minutes to set up each plate according to the layout described in FIG. 22.
x. Once the method is completed empty the remaining contents of the test tubes in the Green Rack into a sealable plastic bag containing a suitable absorbent material. Seal, autoclave and dispose of the bag according to local regulations. Place the empty glass test tubes into the Chloros solution for sterilisation and decontamination before subsequently washing and recycling.

### 7) Incubating the Plates.

Once the plate is complete cover with breathable membrane or plate lid and label clearly. Shake plate vigorously for 30 seconds on microtitre plate shaker (to fully mix the contents of each well) and then incubate at 25° C., unshaken, for approximately 16 hours (or overnight).

### 8) Robot Maintenance

Wash through the pipetting robot with sterile water. (See section 7 i for details).

When leaving for long periods (i.e. >2 days) leave the system filled with 70% ethanol v/v in water. Approximately once a week wash through the system with a suitable disinfectant (i.e. dilute Chloros solution) and then thoroughly with sterile water so that future user's solutions are not contaminated.

Clean the outside of the needle with a soft cloth soaked in ethanol.

Position the needle such that the needle tip is left immersed in sterile water contained in a test tube in the Green Rack, in position A1. (Once switched off the needle and robot arm can be moved manually taking care not to bend or damage the needle. Its position will re-set automatically when commencing a new method.)

### 9) Plate Reading and Data Analysis

For plate reading and analysis refer to the protocol appropriate for the plate reader used. i.e. Tecan Ultra or BMG Polar Star.

### 10) Disposal

When all results processing is completed, contain the used micro-plates within a sealed plastic bag containing a suitable absorbent material. Seal, autoclave and dispose of according to local regulations.

### EXAMPLE 4

In addition to the RAD54 vectors described in the previous Examples, the inventors constructed an FF18984 reporter strain containing a replicative plasmid harbouring an RNR regulatory element. The vectors pGenRNR2 or pGenRNR3 are shown in FIGS. 24 and 25, respectively, and contain the upstream non-coding DNA sequence of the RNR2 gene or RNR3 gene fused to the yeast-enhanced Aequorea victoria GFP gene. A control FF18984 strain containing an identical plasmid except that 2 base pairs have been removed at the start of the GFP gene, such that no GFP is made, was also examined.

pGenRNR2

In order to test the brightness of the reporter for RNR2 constructs, similar methodology was used as for RAD54 constructs, as described in the previous Examples, but using instead pGENRNR2.

FIG. 28 illustrates results obtained in a yeast test strain transformed with pGenRNR2. Brightness/fluorescence was induced by the genotoxic compound MMS. The inventors believe this compound activates the RNR2 regulatory element which in turn results in the expression of GFP and thereby produces a signal measurable according to the method of the invention

pGenRNR3

In order to test the brightness of the reporter for RNR3 constructs, similar methodology was used as for RAD54 constructs, as described in the previous Examples, but using instead pGENRNR3.

FIG. 29 illustrates data similar to that shown in FIG. 28, except that the yeast strain was transformed with pGenRNR3.

FIGS. 30 to 32 show graphs of the response of RNR3—GFP reporter to MMS, 1,2-dimethylhydrazine (2HCl), and EMS, respectively. In each case, it will be seen that the fluorescence induction by the reporter increases with increasing concentration of test compound.

### EXAMPLE 5

The inventors also constructed a vector according to the first aspect, using as backbone, the vector pRS316, which is a centromeric plasmid. pRS316 is URA3 selected. FIG. 39 shows empty pRS316 vector compared with pRS316 containing the RAD54-GFP reporter cassette plus the kanMX module, compared with pRS316 containing the RAD54-GFP cassette but not kanMX. It will be appreciated that pRS316 containing the RAD54-GFP cassette but not kanMX gives a brighter reporter signal. Furthermore, it will be appreciated that the brightness values are lower than pWDH445, since pRS316 is a centromeric plasmid i.e. at a low copy number.

1. A recombinant vector comprising a recombinant DNA molecule comprising a DNA sequence that encodes a light emitting reporter protein, which DNA sequence is operatively linked to a regulatory element arranged to activate expression of the DNA sequence in response to DNA damage, wherein when used to transform a cell, the vector does not substantially alter the sensitivity of the cell to geneticin, when compared to the sensitivity of the cell which has not been transformed with the vector.

2. The recombinant vector according to claim 1, wherein the light emitting reporter protein is Green Fluorescent Protein or a light-emitting derivative thereof.

3. The recombinant vector according to claim 2, wherein the recombinant DNA molecule comprises a DNA sequence encoding the S65T derivative of Green Fluorescent Protein.

4. The recombinant vector according to claim 3, wherein the recombinant DNA molecule comprises a DNA sequence encoding a Yeast Enhanced derivative of Green Fluorescent Protein.

5. The recombinant vector according to any preceding claim, wherein the vector is designed to autonomously replicate in a cell.

6. The recombinant vector according to claim 5, wherein the vector contains DNA from the 2μ plasmid.

7. The recombinant vector according to any one of claims 1 to 4, wherein the vector is designed to integrate in the genomic DNA of a cell.

8. The recombinant vector according to claim 7, wherein the vector comprises DNA encoding for part of the HO gene from chromosome IV of a yeast.

9. The recombinant vector according to any one of claims 7, wherein the vector comprises sequences from the ribosomal DNA array of S. cerevisiae.

10. The recombinant vector according to claim 1, wherein the regulatory element comprises a yeast RAD54 gene.

11. The recombinant vector according to claim 1, wherein the regulatory element comprises a yeast RNR regulatory element.

12. The recombinant vector according to claim 11, wherein the regulatory element comprises the RNR2 or RNR3 gene.

13. The recombinant vector according to claim 1, wherein the vector is a pFA vector.

14. A recombinant vector according to claim 1, comprising a light emitting reporter protein, a regulatory element and a non-functional kanMX module.

15. A recombinant vector according to claim 14, wherein the vector comprises a non-functional kanMX3 module.

16. A recombinant vector according to claim 15, wherein the KanMX3 module is disrupted with a deletion, substitution or addition.

17. A recombinant vector according to claim 1 comprising the vector of FIG. 15 or a functional derivative thereof.

18. A recombinant vector according to claim 1 comprising the vector of FIG. 24 or a functional derivative thereof.

19. A recombinant vector according to claim 1 comprising the vector of FIG. 25 or a functional derivative thereof.

20. A recombinant vector according to claim 1 comprising the vector of FIG. 34 or a functional derivative thereof.

21. A recombinant vector according to claim 1 comprising the vector of FIG. 36 or FIG. 38 or a functional derivative thereof.

22. A cell containing a recombinant vector according to claim 1.

23. The cell according to claim 22, wherein the cell is a yeast.

24. The yeast according to claim 23, wherein the yeast is Saccharomyces cerevisiae.

25. The yeast according to claim 24 which is FF18984 or Y486 in haploid form.

26. A method of detecting the presence of an agent that causes or potentiates DNA damage, the method comprising subjecting a cell according to any one of claims 22-25 to an agent and monitoring the expression of the light emitting reporter protein from the cell, wherein an increase of the expression in the presence of the agent indicates that the agent causes or potentiates DNA damage.

27. The method according to claim 26, wherein the agent is further screened to assess whether it is safe to expose a living organism to the agent.

28. The method according to claim 26, wherein the agent is a candidate medicament, food additive or cosmetic.

29. The method according to claim 26, wherein the agent is a contaminant of water supplies.

30. The method according to claim 26, wherein the agent is a contaminant of industrial effluents.

31. The method according to claim 26, wherein expression of the light emitting reporter protein is measured from a whole cell.

32. The method according to claim 26, wherein the light emitting reporter protein is Green Fluorescent Protein.

33. The method according to claim 32 comprising growing cells transformed with a recombinant vector according to claim 1, incubating the cells with the agent for a predetermined time and monitoring the expression of the Green Fluorescent Protein directly from a sample of the cells.

34. The method according to claim 26, wherein the cells are grown in a low fluorescence growth medium.

35. The method according to claim 34, wherein the low fluorescence growth medium is F1 medium.

36. A method of generating a recombinant vector, the method comprising the steps of: —
(i) providing a vector backbone with a DNA sequence that encodes a light emitting reporter protein;
(ii) operatively linking the DNA sequence to a regulatory element arranged to activate expression of the DNA sequence in response to DNA damage;
(iii) providing the vector backbone with a selectable marker arranged to confer resistance to geneticin; and
(iv) rendering the selectable marker non-functional, wherein when used to transform a cell, the vector does not substantially alter the sensitivity of the cell to geneticin, when compared to the sensitivity of the cell which has not been transformed with the vector.


SEQUENCE LISTING (9 sequences)

SEQ ID NO: 1 - DNA, 10661 bp, Unknown
  Recombinant Vector “pGen001”
gatccaagctatctactgagatttctggctcttttgttgtactgtcacctaaccacagaccaagcatccaagccatactttttacagcaggagttacaaggtcactacgtccagtgagaaatttagataaaacaccatttcctgcgagtactggaccaaatcttatgcagctagaaattctcaattgagcatcaagataatccaaatctctaacttcaatgtcaaagttgaaatattctcctttagagcgctccatttcttctatgaagcgttttgcggcaaactcaccttcaactgtcattgggaatgtcttatgatggttttttggaattattattatcctaccatcaagcgtctgacattgctgcagatttctccatctcactttatatttggtggcatttctaccacttttttccaacagtggtttggtagggaccctgactgacaatttatgacctgcagtacattgtaatgcaagacgctgataaactgttctacgcctgggatctaacctaccaggttcaccttcaaaagctctgtgtttggttttttgctgtatattatagattttctgatagccctgtgtgacatttatgacgcgggcagcggagccatctgcgcacataacgtaagagttagccgtgacgtttgcgatgtctttaatttcaccgttagccatcagaatagtcgtgttttcagaaagcattttgatccgacatacgatgacctcaatgatttagattatgtgttgcacttttatagacctaccaaaaatccagtgcgtacactaatactttcataaagatacctgaaacaataaccagaaagatcggcaaaaaaattttttttctttgccgagatcacaaacctactatgacgaaaaagcttgaagtttagatgagtaaggaaaatacaagtgacgcttttatatggtgcaaggaacaaaaactaaaaacaacaaggcaaatgtggatctgtcatgtatggcaacgacagcaggatggctcacaaaaaaagacaaaaaaaactaaggcaaaagaacaaagctcctctcctgctcaagaaacgtattgttgaaaaaccaccgtcgtaagaaagtttttctgtgacctataatggtttaaaatcggcccattttttttccctcttttgtggtccagtctttctcatactcgagggaaattcgacacaaacagcggagaagtgtggctaaaccggcaagtgcctgcaagatccacagaactaaccgcacgaactggcggtcagaaaagagcctgttccggaaagagagaaacagagaaacgatcatgatgggaaagcggggattcggcgaagaacgagactggaaagggaaaaagagaaatactggtggaagtattcggacctttggcgaagtccgaacccttgaaacccaaagatgatcgatgattcatttttcaatgcgctacggttcctgccgctcgtgggaaccccacgcaaaacatattattcgcttctctctgctgacaactccggtttacgttataccgtattaggatcactataagggttccttcgggaggaggggggaggggaagaatgtacatcgtcataaggcctttatggtgtgaagtgggttttgcgtggaaaattcgttttcaatgatatagagcccacgcatatacgtacatactagtggccaaaagcgtggggtgggcggacaaagctacactggtaaaatacaggattctatgaacaataacaacaaccagctcacgttgctgaacagccgaggtcagccgatgcaaccgaggtttccaaagtagcatttctgtgctagctatgtctgtaggtttacatttaatggtgcgtggttccagcttcatgtgcttgcatgtgatgtcctgcagatggtaagaagattctgaaagccgcgctaggagaaaaatattctgctcgaagatctgtcctcttaagtagaaagcgtgaaattgttgcgttcttgcattactactcaacgcgtacgcaaatgcgtctactgcacctgcatgataaagcttatgtatcaaaaatttaacatcttgaaaatacacaagtggtgcaaagatgtgtcacgttctggacctgagtggtgccatgtatgctatttaacatgcaaaggggaagacccttccgccttactgcaataataaaaagtattttacgcgttacccaatatagcaaagtttcgcgcaaaaaaaaaaataaaaaacaattacaaacaaaaagaaaaaaaaggaaataatagaagatctaactgaagcgaaggccaaaactcttctcacttgacgtaatagccgatacaaaatctagagcagcaacttttctctttcttcactaaagctgctacgaaagtatagaaaaatcaaacgctcagaacttagctctatttcaaggtaccatatatatttccttataactgatgttaattaactctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataactgcagggcgcgccacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagtatgaggtcgctcttattgaccacacctctaccggcagatccgctagggataacagggtaatatagatctgcccgccgggaaggcgaacccgatcggatgcatcctctctgctgccatgatgctgaagttgtcgttgaacatggttgctgccggcgaggcggtcgagcaggcagtgcaggaggtgttggactcgggagtcagaacgggcgacctgctcggctcgagctccacttcggaggttggcgacgccattgcgcttgcagttaaggaagccttgcgcaggcaatccgcagctggtctgagctagcctcgaggacccttctctttagactattctactcttatgcacgtaaaaaattctaggaaatatgtattaactaggagtaaaataaccggctagtggcattcatatagccgtctgtttacatctacatcacacatttcgagtgtatatctcgcaacgttggcgttaaataggcagtcaatggcccgaccattctatggtgtttaggtcgatgccatctttgtacgtttagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcggggggggggggggttttctttccaattttttttttttcgtcattatagaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaaaaaatttcaaagaaaccggaatcaaaaaaaagaacaaaaaaaaaaaagatgaattgaaaccccccccccccccgatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagag

SEQ ID NO: 2 - DNA, 10661 bp, Unknown; identical to SEQ ID NO: 1
  Recombinant Vector “pGen001”

SEQ ID NO: 3 - DNA, 11712 bp, Unknown
  Recombinant Vector “pGenRNR2”
ggatccgtaccttccagcattgtccttctgagaaaacaaaaatggaagatgttgtgaaaatgcagtaagtgacaataagctagcgacttttcaatgatcgcagattaagtatatatacactttaggtctccttctcttctttactagctacgaaaaaacaaagaaaaaaaaaacaaaatagagggaaaaaagatatacgtataaaaatatgctccaggggaggttcgaactctcgaccttcagattatgagactgacgctcttcctactgagctactgaagcactatttcttgttccaaccgaggaataatactagacctgtagaaaagtaccgcttatctaatatccaactttttcttctgttccctttagtgaattcaagatgaaggaacataacagaacggaaaacaaaaaaaaagggaagaaaaaagatatcaccaccaattggcacgctcgcaacggtttcgggccagattcatggccctgtggggtagaagtgcctacagacaaaaaaccgttacgtcccgcctcaccaagcatcgacacacaagccctctcaacctccttaatttctttatctttttttcccttcacctctgtgctctttttttagccacagctgtgcattacgcttgctttgcttgttttgttctctttcgccatgccatattactacttttctacttactggcacgaggccgcgcaagccagatccaagcacgccagaaagtgcggtgtactggtgcaaccttttatcgatttctcatcatatcgctatcgcaacgggcatgggcgacgaaaaagccaatctaaagcgttgtcttcctcgttcctcgcgcacgcgattttgcgttctgcataggaagccgaagtcgaacaagaagcaggcaaagtttagagcactgcccctccgcactcaaaaaagaaaaaactaggaggaaaataaaattctcaaccacacaaacacataaacacatacaaatacaaatacaagcttatttacttgacatcgcgcgatcttccactattcagcgccgtccgccctctctcgtgttttttgtttacgcgacaactatgcgaaatccggagcaacgggcaaccgtttggggaaagaccacacccacgcgcgatcgccatggcaacgaggtcgcacacgccccacacccagacctccctgcgagcgggcatgggtacaatgtccccgttgccacagagaccacttcgtagcacagcgcagagcgtagcgtgttgttgctgctgacaaaagaaaatttttcttagcaaagcaaaggaggggaagcacgggcagatagcaccgtaccatacccttggaaactcgaaatgaacgaagcaggaaatgagagaatgagagttttgtaggtatatatagcggtagtgtttgcgcgttaccatcatcttctggatctatctattgttcttttcctcatcactttcccctttttcgctcttcttcttgtcttttatttctttcttttttttaattgttccctcgattggctatctaccaaagaatccaaacttaatacacgtatttatttgtccaattaccatgttaattaactctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataactgcagggcgcgccacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagtatgaggtcgctcttattgaccacacctctaccggcagatccgctagggataacagggtaatatagatctgcccgccgggaaggcgaacccgatcggatgcatcctctctgctgccatgatgctgaagttgtcgttgaacatggttgctgccggcgaggcggtcgagcaggcagtgcaggaggtgttggactcgggagtcagaacgggcgacctgctcggctcgagctccacttcggaggttggcgacgccattgcgcttgcagttaaggaagccttgcgcaggcaatccgcagctggtctgagctagcctcgaggacccttctctttagactattctactcttatgcacgtaaaaaattctaggaaatatgtattaactaggagtaaaataaccggctagtggcattcatatagccgtctgtttacatctacatcacacatttcgagtgtatatctcgcaacgttggcgttaaataggcagtcaatggcccgaccattctatggtgtttaggtcgatgccatctttgtacagcttgcctcgtccccgccgggtcacccggccagcgacatggaggcccagaataccctccttgacagtcttgacgtgcgcagctcaggggcatgatgtgactgtcgcccgtacatttagagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgactagggttgctgccatcggcctcgctcgcgtctttgccggatagcaagagcgcctttggcctctacgagccctgccacggctctgcgcccgatctgcccgccgggaaggcgaacccgatcggatgcatcctctctgctgccatgatgctgaagttgtcgttgaacatggttgctgccggcgaggcggtcgagcaggcagtgcaggaggtgttggactcgggagtcagaacgggcgacctgctcggctcgagctccacttcggaggttggcgacgccattgcgcttgcagttaaggaagccttgcgcaggcaatccgcagctggtctgagctagcctcgaggacccttctctttagactattctactcttatgcacgtaaaaaattctaggaaatatgtattaactaggagtaaaataaccggctagtggcattcatatagccgtctgtttacatctacatcacacatttcgagtgtatatctcgcaacgttggcgttaaataggcagtcaatggcccgaccattctatggtgtttaggtcgatgccatctttgtacgtttagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcggggggggggggggttttctttccaattttttttttttcgtcattatagaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaaaaaatttcaaagaaaccggaatcaaaaaaaagaacaaaaaaaaaaaagatgaattgaaaccccccccccccccgatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtaga

SEQ ID NO: 4 - DNA, 8840 bp, Unknown
  Recombinant Vector “pGenRNR3”
ggatccagaaacaagagaaggtaacaagcacataaaaaatcagcacatacgtacatacataagaatgaatcgcacgcacgcgtaaacatttatcatttaatcttcagttgttagataaaaaaaaaaagaaaagaaaagaaagtgaaggcttgtttcagtttgaactaggtagcagagcaagccctcgttcttggctgctaattttcctaaagtagtaaaaaaagccaagttatctgcctacggttgtcacagcaacattgcgtgccgttgttcttttgtttttttttttttttttttttcgtggttgtcgcagcaacgacacctaggcgctgctcaaaggggcaaaaacccggttgccatggcgaggaccaaacgacaagatgggaaaaaaacaatagtctattgttaaatcgtaatactgtattgtgagatgctgacgcgtttcgtttttcgtgtcagcgttctttatattgtttcgtgttctgctgcaaaacgtatataaacgcactgctattttgccttcttttgccttcttccttgcctttctctcatctcatatccaagttgaaataaatatgacaagcaagaatagcagcagcaataaatcaaatactcccacacaaatgttaattaactctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataactgcagggcgcgccacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagtatgaggtcgctcttattgaccacacctctaccggcagatccgctagggataacagggtaatatagatctgcccgccgggaaggcgaacccgatcggatgcatcctctctgctgccatgatgctgaagttgtcgttgaacatggttgctgccggcgaggcggtcgagcaggcagtgcaggaggtgttggactcgggagtcagaacgggcgacctgctcggctcgagctccacttcggaggttggcgacgccattgcgcttgcagttaaggaagccttgcgcaggcaatccgcagctggtctgagctagcctcgaggacccttctctttagactattctactcttatgcacgtaaaaaattctaggaaatatgtattaactaggagtaaaataaccggctagtggcattcatatagccgtctgtttacatctacatcacacatttcgagtgtatatctcgcaacgttggcgttaaataggcagtcaatggcccgaccattctatggtgtttaggtcgatgccatctttgtacgtttagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcggggggggggggggttttctttccaattttttttttttcgtcattatagaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaaaaaatttcaaagaaaccggaatcaaaaaaaagaacaaaaaaaaaaaagatgaattgaaaccccccccccccccgatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtaga

SEQ ID NO: 5 - DNA, 3610 bp, Saccharomyces cerevisiae
agattctgcgccagcaagtcgtctccgagggggggcccaccgctaccatacagtcaaggtttaactacgcatgggggctgatcaaatccactgacgtgaatgacgaaaggcttggtgtgaaaatcctcacagacatttacaaagaggccgagtcccgtagacgagaatgcctatattatctgaccataggttgctacaaactcggtgaatactctatggcgaagagatatgtagacactttatttgagcatgagcgtaataacaagcaggtgggcgctttgaagagtatggtagaggataagatccagaaggaaacactcaagggtgttgtcgtcgctggaggcgtactagccggcgctgtggccgtggctagtttcttcttaagaaacaagagaaggtaacaagcacataaaaaatcagcacatacgtacatacataagaatgaatcgcacgcacgcgtaaacatttatcatttaatcttcagttgttagataaaaaaaaaaagaaaagaaaagaaagtgaaggcttgtttcagtttgaactaggtagcagagcaagccctcgttcttggctgctaattttcctaaagtagtaaaaaaagccaagttatctgcctacggttgtcacagcaacattgcgtgccgttgttcttttgtttttttttttttttttttttcgtggttgtcgcagcaacgacacctaggcgctgctcaaaggggcaaaaacccggttgccatggcgaggaccaaacgacaagatgggaaaaaaacaatagtctattgttaaatcgtaatactgtattgtgagatgctgacgcgtttcgtttttcgtgtcagcgttctttatattgtttcgtgttctgctgcaaaacgtatataaacgcactgctattttgccttcttttgccttcttccttgcctttctctcatctcatatccaagttgaaataaatatgacaagcaagaatagcagcagcaataaatcaaatactcccacacaaatgtacgttattaaaagagacggccgcaaagagcccgttcaattcgataaaattacctcccgtatcacccgtttgtcatacggtttagacccaaaccgtattgatgctgttaaggtaacccaacgtattatttctggtgtgtactccggtgttactaccgttgagctggacaatcttgcagctgaaacatgtgcatacatgaccactgtgcaccctgattatgccactctagccgctagaatcgccatctctaacttacataagcaaaccacaaagcaattctccaaagttattgaggatttacacgactggattaacccagctactggaaagcatgctcctatgatttcggacgaaatttacaacattgtcatggaaaacaaagatactttgaactcggccatcgtgtacgatagggatttccagtatacgtatttcggattcaagacactggagcgttcgtacttgctaagactgaacggtgaagtggcagaacgtcctcagcatttggtaatgcgtgtggcgctaggtatccatggtagcgatatcgaatctgtgctgaagacttataatttgatgtcgttaagatacttcactcacgcttccccaactttattcaacgctggtacgccacatcctcaaatgtcttcatgtttcttaattgccatgaaggatgactctatcgaaggtatttatgatactttgaaagaatgtgctatgatttccaaaactgcaggtggtgttggtcttcatatcaacaacatccgttccacaggttcttatatcgctggtaccaacggtacttcaaacgggttgattcctatgattcgtgttttcaataatactgcccgttatgtggaccagggtggtaacaagagacctggtgctttcgcccttttcttggagccatggcatgcagatatcttcgactttgtcgatatcagaaaaacacatggtaaggaagaaattcgtgcaagagatttgttccctgctctatggatccctgatcttttcatgaaacgtgttcaagaggatgggccttggactttgttttcgcccagtgctgccccaggtttagatgatgtgtggggtgatgaatttgaagaactatatactcgttacgaaagagaaggtcgtggtaaaacaattaaagcccaaaagttgtggtatgccattttgcaagcacagacagaaacaggtacacctttcatggtttataaggacgcatgtaacaggaagacaaaccaacagaacttaggtactatcaaatcatctaatttatgttgtgaaatcgtcgaatattcctccccggatgaaactgcagtttgtaatttagcttctattgccctaccagcattcgttgaggtttcagaagatggtaaaactgcaagctataatttcgagagattacacgagattgctaaagtcattactcacaacttgaacagagttatcgaccgtaattactatccagttcccgaggctagaaattcaaatatgaagcatagacctattgctcttggtgtccagggtttggccgatacttatatgatgttgcgtctaccctttgaatcggaagaagctcaaactctaaacaaacaaatcttcgaaactatttaccatgctactcttgaagcctcctgtgaattggcccaaaaagaaggtaaatattctacttttgaaggttctccagcttctaagggtattttacaattcgatatgtggaacgctaaaccatttggcatgtgggattgggaaaccttaagaaaggacattgttaaacatgggttaagaaactctttgactatggcaccaatgccaaccgcctcaacttcccaaattcttggttataatgaatgcttcgaaccagtgacctcaaacatgtactctcgtcgtgtcctgtctggtgaattccaagttgttaatccatatttactacgtgatttagtcgacctgggtatttgggatgatagtatgaaacaatatctaattacacaaaatggttctattcaaggcttaccaaatgtgccacaagaattgaaggaattatacaaaaccgtctgggaaatctctcaaaagaccattatcaatatggctgctgatcgtgccatctacatcgatcagtctcattccttgaatcttttcttgcaagcaccatcaatgggtaagattactagtatgcatttctacggttggaagaagggtttaaaaactggtatgtactacttaagaacgcaagccgcctccgctgctattcaatttaccattgatcaagaggttgccgatcaagccgctacacatattgcttccgtctcagaattggatcgtccagtttatgttccaaagggtacaaaattctctgaacaaaaggcggcatctgcgcttaccgaaagctcagataatgagaaggatgcatctccagttccatccgaacaatcatcggtgtcgagtgccatgtcaaatgtgaaattggaagatagtgttgccccagcagttccaacggaaacaataaaagaagattccgacgagaagaaatgtgacatttacaatgaaaaggtgattgcttgtactgctcctactccagaagcttgtgagtcatgttccggttga

SEQ ID NO: 6 - DNA, 892 bp, Saccharomyces cerevisiae
aaattgtgacagctttccagaatggattatttttcctcaaattccttgtcttcctgttttcatctggaccatctccataatgaagccttacatgtttggcacgtagcggaacgtgatcgtcacaaaccgtaaggtagagaccccagattttcgcattttctcttaaactctccattagcttaggatccaagctatctactgagatttctggctcttttgttgtactgtcacctaaccacagaccaagcatccaagccatactttttacagcaggagttacaaggtcactacgtccagtgagaaatttagataaaacaccatttcctgcgagtactggaccaaatcttatgcagctagaaattctcaattgagcatcaagataatccaaatctctaacttcaatgtcaaagttgaaatattctcctttagagcgctccatttcttctatgaagcgttttgcggcaaactcaccttcaactgtcattgggaatgtcttatgatggttttttggaattattattatcctaccatcaagcgtctgacattgctgcagatttctccatctcactttatatttggtggcatttctaccacttttttccaacagtggtttggtagggaccctgactgacaatttatgacctgcagtacattgtaatgcaagacgctgataaactgttctacgcctgggatctaacctaccaggttcaccttcaaaagctctgtgtttggttttttgctgtatattatagattttctgatagccctgtgtgacatttatgacgcgggcagcggagccatctgcgcacataacgtaagagttagccgtgacgtttgcgatgtctttaatttcaccgttagccatcagaatagtcgtgttttcagaaagcat

SEQ ID NO: 7 - DNA, 4582 bp, Saccharomyces cerevisiae
gatctgacgatcacctagcgactctctccaccgtttgacgaggccatttacaaaaacataacgaacgacaagcctactcgaattcgtttccaaactcttttcgaacttgtcttcaactgctttcgcatgaagtacctcccaactacttttcctcacacttgtactccatgactaaaccccccctcccattacaaactaaaatcttacttttattttcttttgccctctctgtcgctctgccttaactacgtatttctcgccgagaaaaacttcaatttaagctattctccaaaaatcttagcgtatattttttttccaaagtgacaggtgccccgggtaacccagttcctcactattttttactgcggaagcggaagcggaaaatacggaaacgcgcgggaacatacaaaacatacaaaatatacctttctcacacaagaaatatatgctacttgcaaaatatcataccaaaaaacttttcacaaccgaaaccaaaaccaacggatatcatacattacactaccaccattcaaactttactactatcctcccttcagtttccctttttctgcctttttcggtgacggaaatacgcttcagagaccctaaagggaaatccatgccataacaggaaagtaacatcccaatgcggactataccaccccaccacactcctaccaataacggtaactattctatgttttcttactcctatgtctattcatctttcatctgactacctaatactatgcaaaaatgtaaaatcatcacacaaaacataaacaatcaaaatcagccatttccgcaccttttcctctgtccactttcaaccgtccctccaaatgtaaaatggcctatcggaatacattttctacatcctaactactataaaacaacctttagacttacgtttgctactctcatggtctcaatactgccgccgacattctgtcccacatactaaatctcttcccgtcattatcgcccgcatccggtgccgtaaatgcaaaacaaataccatctatgtcttccacaccatcattttactatgcctgccaccatccatttgtcttttgcaccatatcttcataacctgtcaccttgaaactacctctgcatgccacctaccgaccaactttcatgttctgtttcgacctacctcttgtaaatgacaaatcacctttttcatcgtatgcaccttattctccacatcacaatgcactattgcttttgctttttcacctgtcatatcctattgctattagatgaaatataataaaaattgtcctccacccataacacctctcactcccacctactgaacatgtctggaccctgccctcatatcacctgcgtttccgttaaactatcggttgcggccatatctaccagaaagcaccgtttcccgtccgatcaactgtagttaagctggtaagagcctgaccgagtagtgtagtgggtgaccatacgcgaaactcaggtgctgcaatctttatttctttttttttttttttttttttttttttttctagtttcttggcttcctatgctaaatcccataactaacctaccattcgattcagaaaaattcgcactatccagctgcactcttcttctgaagagttaagcactccattatgctcattgggttgctactacttgatatgtacaaacaatattctcctccgatattcctacaaaaaaaaaaaaaaaaacactccggttttgttctcttccctccatttccctctcttctacggttaatactttcctcttcgtctttttctacaccctcgtttagttgcttcttattccttcccgctttcctgcactaacattttgccgcattacactatatgatcgtagtacatcttacaactccgcataccgcgtcgccgcgtcgccgcgtcgccaaaaatttacttcgccaaccattccatatctgttaagtatacatgtatatattgcactggctattcatcttgcacttttcctctttcttcttcccagtagcctcatccttttacgctgcctctctggaacttgccatcatcattccctagaaactgccatttacttaaaaaaaaaaaaaaaaaaaaaatgtccccactgttcactgttcactgttcacttgtctcttacatctttcttggtaaaatcgtagttcgtagtattttttttcatatcaaaggcatgtcctgttaactataggaaatgagcttttctcaattctctaaacttatacaagcacctcatgtttgccgctctgatggtgcggaaaaaactgctccatgaagcaaactgtccgggcaaatcctttcacgctcgggaagctttgtgaaagcccttctctttcaacccatctttgcaacgaaaaaaaaaaaaaaaataaaaaataaaaagaccaaatagtaaatagtaacttacatacattagtaaatggtacactcttacacactatcatcctcatcgtatattataatagatatatacaatacatgtttttacccggatcatagaattcttaagacaaataaaatttatagagacttgttcagtctacttctctctaaactaggccccggctcctgccagtacccacttagaaagaaataaaaaacaaatcagacaacaaaggcttaatctcagcagatcgtaacaacaaggctactctactgcttacaataccccgttgtacatctaagtcgtatacaaatgatttatccccacgcaaaatgacattgcaattcgccagcaagcacccaaggcctttccgccaagtgcaccgttgctagcctgctatggttcagcgacgccacaaggacgccttattcgtatccatctatattgtgtggagcaaagaaatcaccgcgttctagcatggattctgacttagaggcgttcagccataatccagcggatggtagcttcgcggcaatgcctgatcagacagccgcaaaaaccaattatccgaatgaactgttcctctcgtactaagttcaattactattgcggtaacattcatcagtagggtaaaactaacctgtctcacgacggtctaaacccagctcacgttccctattagtgggtgaacaatccaacgcttaccgaattctgcttcggtatgataggaagagccgacatcgaagaatcaaaaagcaatgtcgctatgaacgcttgactgccacaagccagttatccctgtggtaacttttctggcacctctagcctcaaattccgagggactaaaggatcgataggccacactttcatggtttgtattcacactgaaaatcaaaatcaagggggcttttacccttttgttctactggagatttctgttctccatgagccccccttaggacatctgcgttatcgtttaacagatgtgccgccccagccaaactccccacctgacaatgtcttcaacccggatcagccccgaatgggaccttgaatgctagaacgtggaaaatgaattccagctccgcttcattgaataagtaaagaaactataaaggtagtggtatttcactggcgccgaagctcccacttattctacaccctctatgtctcttcacaatgtcaaactagagtcaagctcaacagggtcttctttccccgctgattctgccaagcccgttcccttggctgtggtttcgctagatagtagatagggacagtgggaatctcgttaatccattcatgcgcgtcactaattagatgacgaggcatttggctaccttaagagagtcatagttactcccgccgtttacccgcgcttggttgaatttcttcactttgacattcagagcactgggcagaaatcacattgcgtcaacatcactttctgaccatcgcaatgctatgttttaattagacagtcagattccccttgtccgtaccagttctaagttgatcgttaattgtagcaagcgacggtctacaagagacctaccaaggccgtctacaacaaggcacgcaagtagtccgcctagcagagcaagccccaccaagcagtccacaagcacgcccgctgcgtctgaccaaggccctcactacccgacccttagagccaatccttatcccgaagttacggatctattttgccgacttcccttatctacattattctatcaactagaggctgttcaccttggagacctgctgcggttatcagtacgacctggcatgaaaactattccttcctgtggattttcacgggccgtcacaagcgcaccggagccagcaaaggtgctggcctcttccagccataagaccccatctccggataaaccaattccggggtgataagctgttaagaagaaaagataactcctcccagggctcgcgccgacgtctccacattcagttacgttaccgtgaagaatccatatccaggttccggaatcttaaccggattccctttcgatggtggcctgcataaaatcaggcctttgaaacggagcttccccatctcttaggatcgactaacccacgtccaactgctgttgacgtggaacctttccccacttcagtcttcaaagttctcatttgaatatttgctactaccaccaagatc

SEQ ID NO: 8 - DNA, 7515 bp, Unknown
  Recombinant Vector “pGenIn012”
gaacgcggccgccagctgaagcttcgtacgctgcaggtcgacggatcaaaattgtgacagctttccagaatggattatttttcctcaaattccttgtcttcctgttttcatctggaccatctccataatgaagccttacatgtttggcacgtagcggaacgtgatcgtcacaaaccgtaaggtagagaccccagattttcgcattttctcttaaactctccattagcttaggatccaagctatctactgagatttctggctcttttgttgtactgtcacctaaccacagaccaagcatccaagccatactttttacagcaggagttacaaggtcactacgtccagtgagaaatttagataaaacaccatttcctgcgagtactggaccaaatcttatgcagctagaaattctcaattgagcatcaagataatccaaatctctaacttcaatgtcaaagttgaaatattctcctttagagcgctccatttcttctatgaagcgttttgcggcaaactcaccttcaactgtcattgggaatgtcttatgatggttttttggaattattattatcctaccatcaagcgtctgacattgctgcagatttctccatctcactttatatttggtggcatttctaccacttttttccaacagtggtttggtagggaccctgactgacaatttatgacctgcagtacattgtaatgcaagacgctgataaactgttctacgcctgggatctaacctaccaggttcaccttcaaaagctctgtgtttggttttttgctgtatattatagattttctgatagccctgtgtgacatttatgacgcgggcagcggagccatctgcgcacataacgtaagagttagccgtgacgtttgcgatgtctttaatttcaccgttagccatcagaatagtcgtgttttcagaaagcattttgatccgacatacgatgacctcaatgatttagattatgtgttgcacttttatagacctaccaaaaatccagtgcgtacactaatactttcataaagatacctgaaacaataaccagaaagatcggcaaaaaaattttttttctttgccgagatcacaaacctactatgacgaaaaagcttgaagtttagatgagtaaggaaaatacaagtgacgcttttatatggtgcaaggaacaaaaactaaaaacaacaaggcaaatgtggatctgtcatgtatggcaacgacagcaggatggctcacaaaaaaagacaaaaaaaactaaggcaaaagaacaaagctcctctcctgctcaagaaacgtattgttgaaaaaccaccgtcgtaagaaagtttttctgtgacctataatggtttaaaatcggcccattttttttccctcttttgtggtccagtctttctcatactcgagggaaattcgacacaaacagcggagaagtgtggctaaaccggcaagtgcctgcaagatccacagaactaaccgcacgaactggcggtcagaaaagagcctgttccggaaagagagaaacagagaaacgatcatgatgggaaagcggggattcggcgaagaacgagactggaaagggaaaaagagaaatactggtggaagtattcggacctttggcgaagtccgaacccttgaaacccaaagatgatcgatgattcatttttcaatgcgctacggttcctgccgctcgtgggaaccccacgcaaaacatattattcgcttctctctgctgacaactccggtttacgttataccgtattaggatcactataagggttccttcgggaggaggggggaggggaagaatgtacatcgtcataaggcctttatggtgtgaagtgggttttgcgtggaaaattcgttttcaatgatatagagcccacgcatatacgtacatactagtggccaaaagcgtggggtgggcggacaaagctacactggtaaaatacaggattctatgaacaataacaacaaccagctcacgttgctgaacagccgaggtcagccgatgcaaccgaggtttccaaagtagcatttctgtgctagctatgtctgtaggtttacatttaatggtgcgtggttccagcttcatgtgcttgcatgtgatgtcctgcagatggtaagaagattctgaaagccgcgctaggagaaaaatattctgctcgaagatctgtcctcttaagtagaaagcgtgaaattgttgcgttcttgcattactactcaacgcgtacgcaaatgcgtctactgcacctgcatgataaagcttatgtatcaaaaatttaacatcttgaaaatacacaagtggtgcaaagatgtgtcacgttctggacctgagtggtgccatgtatgctatttaacatgcaaaggggaagacccttccgccttactgcaataataaaaagtattttacgcgttacccaatatagcaaagtttcgcgcaaaaaaaaaaataaaaaacaattacaaacaaaaagaaaaaaaaggaaataatagaagatctaactgaagcgaaggccaaaactcttctcacttgacgtaatagccgatacaaaatctagagcagcaacttttctctttcttcactaaagctgctacgaaagtatagaaaaatcaaacgctcagaacttagctctatttcaaggtaccatatatatttccttataactgatgttaattaactctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataactgcagggcgcgccacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagtatgaggtcgctcttattgaccacacctctaccggcagatccgctagggataacagggtaatatagatctgcccgccgggaaggcgaacccgatcggatgcatcctctctgctgccatgatgctgaagttgtcgttgaacatggttgctgccggcgaggcggtcgagcaggcagtgcaggaggtgttggactcgggagtcagaacgggcgacctgctcggctcgagctcgaattcatcgatgatatcagatccactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgaatatcattgagaagctgcattttttttttttttttttttttttttttttatatatatttcaaggatataccattgtaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacaaaaaaaaaaaaaaaaaaaaaaaaaaaaaaaaaaaaaaaaaaaaaaaaaatgcagcgtcacatcggataataatgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 9 - DNA, 12093 bp, Unknown
  Recombinant Vector “pGenIn022A”
gaacgcggccgccagctgaagcttcgtacgctgcaggtcgacggatcaaaattgtgacagctttccagaatggattatttttcctcaaattccttgtcttcctgttttcatctggaccatctccataatgaagccttacatgtttggcacgtagcggaacgtgatcgtcacaaaccgtaaggtagagaccccagattttcgcattttctcttaaactctccattagcttaggatctgacgatcacctagcgactctctccaccgtttgacgaggccatttacaaaaacataacgaacgacaagcctactcgaattcgtttccaaactcttttcgaacttgtcttcaactgctttcgcatgaagtacctcccaactacttttcctcacacttgtactccatgactaaaccccccctcccattacaaactaaaatcttacttttattttcttttgccctctctgtcgctctgccttaactacgtatttctcgccgagaaaaacttcaatttaagctattctccaaaaatcttagcgtatattttttttccaaagtgacaggtgccccgggtaacccagttcctcactattttttactgcggaagcggaagcggaaaatacggaaacgcgcgggaacatacaaaacatacaaaatatacctttctcacacaagaaatatatgctacttgcaaaatatcataccaaaaaacttttcacaaccgaaaccaaaaccaacggatatcatacattacactaccaccattcaaactttactactatcctcccttcagtttccctttttctgcctttttcggtgacggaaatacgcttcagagaccctaaagggaaatccatgccataacaggaaagtaacatcccaatgcggactataccaccccaccacactcctaccaataacggtaactattctatgttttcttactcctatgtctattcatctttcatctgactacctaatactatgcaaaaatgtaaaatcatcacacaaaacataaacaatcaaaatcagccatttccgcaccttttcctctgtccactttcaaccgtccctccaaatgtaaaatggcctatcggaatacattttctacatcctaactactataaaacaacctttagacttacgtttgctactctcatggtctcaatactgccgccgacattctgtcccacatactaaatctcttcccgtcattatcgcccgcatccggtgccgtaaatgcaaaacaaataccatctatgtcttccacaccatcattttactatgcctgccaccatccatttgtcttttgcaccatatcttcataacctgtcaccttgaaactacctctgcatgccacctaccgaccaactttcatgttctgtttcgacctacctcttgtaaatgacaaatcacctttttcatcgtatgcaccttattctccacatcacaatgcactattgcttttgctttttcacctgtcatatcctattgctattagatgaaatataataaaaattgtcctccacccataacacctctcactcccacctactgaacatgtctggaccctgccctcatatcacctgcgtttccgttaaactatcggttgcggccatatctaccagaaagcaccgtttcccgtccgatcaactgtagttaagctggtaagagcctgaccgagtagtgtagtgggtgaccatacgcgaaactcaggtgctgcaatctttatttctttttttttttttttttttttttttttttctagtttcttggcttcctatgctaaatcccataactaacctaccattcgattcagaaaaattcgcactatccagctgcactcttcttctgaagagttaagcactccattatgctcattgggttgctactacttgatatgtacaaacaatattctcctccgatattcctacaaaaaaaaaaaaaaaaacactccggttttgttctcttccctccatttccctctcttctacggttaatactttcctcttcgtctttttctacaccctcgtttagttgcttcttattccttcccgctttcctgcactaacattttgccgcattacactatatgatcgtagtacatcttacaactccgcataccgcgtcgccgcgtcgccgcgtcgccaaaaatttacttcgccaaccattccatatctgttaagtatacatgtatatattgcactggctattcatcttgcacttttcctctttcttcttcccagtagcctcatccttttacgctgcctctctggaacttgccatcatcattccctagaaactgccatttacttaaaaaaaaaaaaaaaaaaaaaatgtccccactgttcactgttcactgttcacttgtctcttacatctttcttggtaaaatcgtagttcgtagtattttttttcatatcaaaggcatgtcctgttaactataggaaatgagcttttctcaattctctaaacttatacaagcacctcatgtttgccgctctgatggtgcggaaaaaactgctccatgaagcaaactgtccgggcaaatcctttcacgctcgggaagctttgtgaaagcccttctctttcaacccatctttgcaacgaaaaaaaaaaaaaaaataaaaaataaaaagaccaaatagtaaatagtaacttacatacattagtaaatggtacactcttacacactatcatcctcatcgtatattataatagatatatacaatacatgtttttacccggatcatagaattcttaagacaaataaaatttatagagacttgttcagtctacttctctctaaactaggccccggctcctgccagtacccacttagaaagaaataaaaaacaaatcagacaacaaaggcttaatctcagcagatcgtaacaacaaggctactctactgcttacaataccccgttgtacatctaagtcgtatacaaatgatttatccccacgcaaaatgacattgcaattcgccagcaagcacccaaggcctttccgccaagtgcaccgttgctagcctgctatggttcagcgacgccacaaggacgccttattcgtatccatctatattgtgtggagcaaagaaatcaccgcgttctagcatggattctgacttagaggcgttcagccataatccagcggatggtagcttcgcggcaatgcctgatcagacagccgcaaaaaccaattatccgaatgaactgttcctctcgtactaagttcaattactattgcggtaacattcatcagtagggtaaaactaacctgtctcacgacggtctaaacccagctcacgttccctattagtgggtgaacaatccaacgcttaccgaattctgcttcggtatgataggaagagccgacatcgaagaatcaaaaagcaatgtcgctatgaacgcttgactgccacaagccagttatccctgtggtaacttttctggcacctctagcctcaaattccgagggactaaaggatcgataggccacactttcatggtttgtattcacactgaaaatcaaaatcaagggggcttttacccttttgttctactggagatttctgttctccatgagccccccttaggacatctgcgttatcgtttaacagatgtgccgccccagccaaactccccacctgacaatgtcttcaacccggatcagccccgaatgggaccttgaatgctagaacgtggaaaatgaattccagctccgcttcattgaataagtaaagaaactataaaggtagtggtatttcactggcgccgaagctcccacttattctacaccctctatgtctcttcacaatgtcaaactagagtcaagctcaacagggtcttctttccccgctgattctgccaagcccgttcccttggctgtggtttcgctagatagtagatagggacagtgggaatctcgttaatccattcatgcgcgtcactaattagatgacgaggcatttggctaccttaagagagtcatagttactcccgccgtttacccgcgcttggttgaatttcttcactttgacattcagagcactgggcagaaatcacattgcgtcaacatcactttctgaccatcgcaatgctatgttttaattagacagtcagattccccttgtccgtaccagttctaagttgatcgttaattgtagcaagcgacggtctacaagagacctaccaaggccgtctacaacaaggcacgcaagtagtccgcctagcagagcaagccccaccaagcagtccacaagcacgcccgctgcgtctgaccaaggccctcactacccgacccttagagccaatccttatcccgaagttacggatctattttgccgacttcccttatctacattattctatcaactagaggctgttcaccttggagacctgctgcggttatcagtacgacctggcatgaaaactattccttcctgtggattttcacgggccgtcacaagcgcaccggagccagcaaaggtgctggcctcttccagccataagaccccatctccggataaaccaattccggggtgataagctgttaagaagaaaagataactcctcccagggctcgcgccgacgtctccacattcagttacgttaccgtgaagaatccatatccaggttccggaatcttaaccggattccctttcgatggtggcctgcataaaatcaggcctttgaaacggagcttccccatctcttaggatcgactaacccacgtccaactgctgttgacgtggaacctttccccacttcagtcttcaaagttctcatttgaatatttgctactaccaccaagatccaagctatctactgagatttctggctcttttgttgtactgtcacctaaccacagaccaagcatccaagccatactttttacagcaggagttacaaggtcactacgtccagtgagaaatttagataaaacaccatttcctgcgagtactggaccaaatcttatgcagctagaaattctcaattgagcatcaagataatccaaatctctaacttcaatgtcaaagttgaaatattctcctttagagcgctccatttcttctatgaagcgttttgcggcaaactcaccttcaactgtcattgggaatgtcttatgatggttttttggaattattattatcctaccatcaagcgtctgacattgctgcagatttctccatctcactttatatttggtggcatttctaccacttttttccaacagtggtttggtagggaccctgactgacaatttatgacctgcagtacattgtaatgcaagacgctgataaactgttctacgcctgggatctaacctaccaggttcaccttcaaaagctctgtgtttggttttttgctgtatattatagattttctgatagccctgtgtgacatttatgacgcgggcagcggagccatctgcgcacataacgtaagagttagccgtgacgtttgcgatgtctttaatttcaccgttagccatcagaatagtcgtgttttcagaaagcattttgatccgacatacgatgacctcaatgatttagattatgtgttgcacttttatagacctaccaaaaatccagtgcgtacactaatactttcataaagatacctgaaacaataaccagaaagatcggcaaaaaaattttttttctttgccgagatcacaaacctactatgacgaaaaagcttgaagtttagatgagtaaggaaaatacaagtgacgcttttatatggtgcaaggaacaaaaactaaaaacaacaaggcaaatgtggatctgtcatgtatggcaacgacagcaggatggctcacaaaaaaagacaaaaaaaactaaggcaaaagaacaaagctcctctcctgctcaagaaacgtattgttgaaaaaccaccgtcgtaagaaagtttttctgtgacctataatggtttaaaatcggcccattttttttccctcttttgtggtccagtctttctcatactcgagggaaattcgacacaaacagcggagaagtgtggctaaaccggcaagtgcctgcaagatccacagaactaaccgcacgaactggcggtcagaaaagagcctgttccggaaagagagaaacagagaaacgatcatgatgggaaagcggggattcggcgaagaacgagactggaaagggaaaaagagaaatactggtggaagtattcggacctttggcgaagtccgaacccttgaaacccaaagatgatcgatgattcatttttcaatgcgctacggttcctgccgctcgtgggaaccccacgcaaaacatattattcgcttctctctgctgacaactccggtttacgttataccgtattaggatcactataagggttccttcgggaggaggggggaggggaagaatgtacatcgtcataaggcctttatggtgtgaagtgggttttgcgtggaaaattcgttttcaatgatatagagcccacgcatatacgtacatactagtggccaaaagcgtggggtgggcggacaaagctacactggtaaaatacaggattctatgaacaataacaacaaccagctcacgttgctgaacagccgaggtcagccgatgcaaccgaggtttccaaagtagcatttctgtgctagctatgtctgtaggtttacatttaatggtgcgtggttccagcttcatgtgcttgcatgtgatgtcctgcagatggtaagaagattctgaaagccgcgctaggagaaaaatattctgctcgaagatctgtcctcttaagtagaaagcgtgaaattgttgcgttcttgcattactactcaacgcgtacgcaaatgcgtctactgcacctgcatgataaagcttatgtatcaaaaatttaacatcttgaaaatacacaagtggtgcaaagatgtgtcacgttctggacctgagtggtgccatgtatgctatttaacatgcaaaggggaagacccttccgccttactgcaataataaaaagtattttacgcgttacccaatatagcaaagtttcgcgcaaaaaaaaaaataaaaaacaattacaaacaaaaagaaaaaaaaggaaataatagaagatctaactgaagcgaaggccaaaactcttctcacttgacgtaatagccgatacaaaatctagagcagcaacttttctctttcttcactaaagctgctacgaaagtatagaaaaatcaaacgctcagaacttagctctatttcaaggtaccatatatatttccttataactgatgttaattaactctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataactgcagggcgcgccacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagtatgaggtcgctcttattgaccacacctctaccggcagatccgctagggataacagggtaatatagatctgcccgccgggaaggcgaacccgatcggatgcatcctctctgctgccatgatgctgaagttgtcgttgaacatggttgctgccggcgaggcggtcgagcaggcagtgcaggaggtgttggactcgggagtcagaacgggcgacctgctcggctcgagctcgaattcatcgatgatatcagatccactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgaatatcattgagaagctgcattttttttttttttttttttttttttttttatatatatttcaaggatataccattgtaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacaaaaaaaaaaaaaaaaaaaaaaaaaaaaaaaaaaaaaaaaaaaaaaaaaatgcagcgtcacatcggataataatgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata